(19) United States
(12) Patent Application Publication
(10) Pub. No.: US 2010/0068214 A1
(43) Pub. Date: Mar. 18, 2010

(54) Identification of Candidate Vaccine Antigens from Dichelobacter Nodosus

(75) Inventors: Julian Rood, Victoria (AU); Keith Al-Hasani, Victoria (AU); John Boyce, Victoria (AU); Dane Parker, Victoria (AU)

Correspondence Address: CROWELL & MORING LLP;INTELLECTUAL PROPERTY GROUP, P.O. BOX 14300, WASHINGTON, DC, 20044-4300, US

(73) Assignee: Monash University, Clayton (AU)

(21) Appl. No.: 12/520,395

(22) PCT Filed: Dec. 19, 2007
(86) PCT No.: PCT/AU2007/001967

§ 371 Date: Nov. 5, 2009

(30) Foreign Application Priority Data
Dec. 21, 2006 (AU) 2006907224

(51) Int. Cl. A61K 39/40; C07K 14/00; C07K 16/00; C07H 21/00; A61K 38/16; A61K 39/02; A61K 31/7052; C12Q 1/68; C40B 40/10; C40B 40/08; C40B 30/04; G01N 33/68; G01N 33/48; A61P 31/04

(52) U.S. Cl. 424/164.1; 530/350; 530/389.5; 536/23.7; 514/12; 424/234.1; 514/44 R; 435/6; 506/18; 506/17; 506/9; 436/86; 436/94

(57) ABSTRACT

This invention relates to a prophylactic and/or therapeutic treatment of footrot, and in particular to a vaccine effective in prophylactic and/or therapeutic treatment of footrot. Specific polypeptides of Dichelobacter nodosus have been identified as vaccine candidates. In some embodiments the polypeptide is expressed more strongly when the bacterium is grown in vivo or is expressed more strongly in the presence of ovine hoof powder. In other embodiments the polypeptide are reactive against sera recovered from animals repeatedly infected with D. nodosus.

### FIELD

This invention relates to a prophylactic and/or therapeutic treatment of footrot, and in particular to a vaccine effective in prophylactic and/or therapeutic treatment of footrot.

### BACKGROUND

All references, including any patents or patent application, cited in this specification are hereby incorporated by reference to enable full understanding of the invention. Nevertheless, such references are not to be read as constituting an admission that any of these documents forms part of the common general knowledge in the art, in Australia or in any other country. The discussion of the references states what their authors assert, and the applicants reserve the right to challenge the accuracy and pertinency of the cited documents.

Footrot is a highly contagious and debilitating disease of the feet of sheep and other ruminants, and is characterized by the separation of the keratinous hoof from the underlying epidermal tissue, resulting in severe lameness and loss of body condition. The consequences of this disease are very significant for the wool and sheep meat industries, and footrot is among the most significant ovine bacterial diseases, causing severe economic losses in most producer countries.

Footrot is dependent on a mixed bacterial infection, but the essential causative agent is Dichelobacter nodosus, a slow-growing, anaerobic, gram-negative rod. D. nodosus exhibits a spectrum of virulence, ranging from virulent strains, which lead to severe underrunning of the horn of the hoof, to benign strains, which cause a self-limiting interdigital dermatitis (Stewart, 1989).

Dichelobacter nodosus, formerly designated as Bacteroides nodosus, is a fastidious and slow-growing Gram-negative aerotolerant anaerobe which is the principal causative agent of footrot in ruminants. In addition to its effects on sheep, D. nodosus is also capable of causing footrot in other animals with cloven hooves, such as cattle, goats and deer; however, apart from ovine footrot only the disease in goats is recognised as a serious clinical problem. Goats are usually less severely affected, and may exhibit different symptoms to sheep infected with the same strain of bacteria.

The virulence of D. nodosus refers to its ability to infect the connective tissue between the horn and flesh of the hoof, and its ability to under-run the horn of the foot. Virulence varies widely between the various strains of D. nodosus. D. nodosus strains of low virulence have poor ability to under-run the hoof horn, and therefore mostly affect the skin between the toes; this is known as benign footrot. Virulent D. nodosus strains rapidly under-run and separate the hoof horn from the foot. Many strains fall somewhere between the benign and virulent extremes. Ovine footrot begins as an interdigital dermatitis before destruction of the epidermal matrix leads to necrotic separation of the hoof from the underlying soft tissue. Infection with D. nodosus requires suitable climatic and environmental conditions, in particular warm weather and moist lush pastures; transmission of the disease ceases in dry conditions which allow dehydration of the interdigital skin. Footrot is virtually always carried into a property and flock by means of a carrier sheep or goat, although cattle and possibly vehicles can also act as carriers. Spread is primarily from foot to foot via pasture or mud; however, moist pastures, laneways and muddy yards are the main areas where footrot is spread. Footrot will therefore spread most rapidly when it is warm and moist, as in spring, early summer and sometimes in autumn.

The treatment of footrot and elimination of infection from a flock requires a combination of methods, including relocation of the sheep to a dry paddock, foot baths, antibiotic therapy, vaccination and effective management, all of which place severe economic strain on farmers. Sheep which have been infected with or exposed to footrot do not develop any significant natural immunity or resistance. In Australia alone the estimated losses due to footrot amount to millions of dollars annually.

Vaccination against D. nodosus has been available for many years (Egerton & Burrell, 1970; Every & Skerman, 1982), but confers only short-term immunity. Vaccines are typically composed of whole cells or purified fimbriae, which provide homologous protection against the serogroup infecting the flock. However, the ability of D. nodosus to undergo serogroup conversion and the observation that reservoirs of serogroups are found in infected hooves highlights the need for heterologous protection (Claxton et al., 1983, Zhou & Hickford, 2000a, Kennan et al., 2003).

Classification of strains of D. nodosus into serogroups and subgroups is based on agglutinogens which are present on surface filaments, which are called pili or fimbriae. These terms are used interchangeably herein. The pili induce a high level of protection in vaccinated sheep against homologous serogroups of D. nodosus. Since there are at least 10 serogroups, the current commercially-available vaccines contain killed whole cells of 8-10 well-piliated strains representative of most of the known serogroups (Claxton et al, 1983). Multivalent recombinant fimbrial vaccines have also been prepared by overexpression of each of the nine fimbrial subunit genes in Pseudomonas aeruginosa.

The currently-available vaccines suffer from a number of disadvantages:
(a) they provide only a 60-80% protection rate;
(b) multiple immunizations during the wet season are required;
(c) they are more expensive than other vaccines used in sheep, partly because of the need for the incorporation of multiple strains into the vaccine and partly because of the fastidious growth requirements and relatively sparse growth of D. nodosus in liquid medium;
(d) pilus expression tends to be variable and, especially with some strains, the irreversible loss of pili from cells following subculture in liquid medium is a major problem to manufacturers;
(e) there is a physical limit to the number of strains which can be incorporated into commercial footrot vaccines, and the efficacy of the whole vaccine could decrease if additional strains need to be incorporated;
(f) antigenic competition has limited the application of these vaccines, so that the most effective strategies are based on univalent and divalent formulations;
(g) multivalent whole bacterial cell vaccines cause clinical problems because they cause large granulomas at the injection site; and
(h) the dominant serogroup causing disease varies from country to country, region to region, and property to property.

Although homologous protection is superior to that obtained against heterologous strains, some cross-protection against heterologous serogroups has been obtained with whole cell vaccines (Thorley and Egerton 1981; Stewart et al 1983; Stewart et al 1985); however, this is not statistically significant.

The surface of Gram-negative bacteria is critical for the interaction of the bacterium with the host cell environment, as it mediates nutrient uptake and secretion of toxins and other products, and is involved in avoidance of the host immune system (Niemann et al., 2004). Furthermore, it is the surface molecules of bacteria which are detected by the host immune system, and it is likely to be these molecules against which the host must mount a protective response. Indeed, outer membrane proteins have been shown to be important for conferring protective immunity in a range of models of infection (Brown et al., 2001, Frazer et al., 2006). Outer membrane proteins are also known to promote adherence to host cell surfaces, and are therefore likely to be involved in D. nodosus virulence (Boyle and Finlay, 2003).

The only experimentally validated vaccine against D. nodosus consists of purified fimbriae, which can only protect against individual serogroups (Stewart, 1989b). Although the protection conferred by fimbrial vaccines is serogroup-specific, low levels of protection, which are not statistically significant, are observed against heterologous challenge, and antibodies raised against fimbriae from one serogroup cross-react with heterologous serogroups (Stewart et al., 1985a).

There have been attempts to identify non-fimbrial antigens which will provide cross-protection against a range of serogroups. For example, U.S. Pat. No. 4,737,363 and PCT/AU88/00176 (WO88/09668) by Commonwealth Scientific and Industrial Research Organisation disclose vaccines respectively comprising an extracellular serine protease and a basic serine protease of D. nodosus, in which the enzymes used in as antigens are found in virulent but not benign strains of D. nodosus. PCT/AU91/00366 (WO92/03553) by Daratech Pty Ltd discloses a vaccine comprising a recombinant secreted outer membrane protein of D. nodosus. However, these vaccines have not been made commercially available, and it is our understanding that the vaccine disclosed in PCT/AU91/00366 proved not to be protective.

There is therefore a need in the art for the identification of suitable antigens which can form the basis of vaccines suitable for use in sheep. In particular there is a need for a vaccine which provides heterologous protection, i.e. protection against more than one serogroup of D. nodosus.

### SUMMARY

In a first aspect, the invention provides an isolated immunogenic D. nodosus polypeptide, expressed from a gene locus selected from the group consisting of the proteins encoded by the gene loci listed in Table 3, Table 5 or Table 6, or a biologically active or immunogenic fragment, derivative, or variant thereof, with the proviso that the polypeptide is not AprV2 (DNO_{—}1167; SEQ ID NO: 189), AprV5 (DNO_{—}0603; SEQ ID NO: 183), BprV (DNO_{—}0605; SEQ ID NO: 184), a polypeptide encoded by the Omp1 locus, or a fimbrial polypeptide encoded by the fimA fimbrial subunit gene.

The polypeptide may be a recombinant or synthetic polypeptide, or may be extracted from D. nodosus bacteria or from D. nodosus culture supernatant or from biological material infected with D. nodosus.

Polypeptides which are expressed at the bacterial cell surface, secreted, encoded by nucleic acid sequences comprise regions of atypical nucleotide composition, found in strains associated with virulence, or exhibit similarity to putative vaccine candidate proteins from other bacterial species are considered likely to be useful as antigens for use in vaccines against D. nodosus, and are used in some embodiments of the invention.

In some embodiments, the polypeptide is selected from the group consisting of the proteins whose corresponding gene loci are listed in Table 3, Table 5 or Table 6.

In some embodiments, the polypeptide is selected from the group consisting of:
a) a protein involved in transport of molecules across a D. nodosus outer membrane;
b) a protein involved in iron uptake;
c) a protein involved in transport of nutrients;
d) a protein involved in resistance of D. nodosus to heavy metals; and
e) a protein involved in synthesis or structural stability of the outer membrane.

In some embodiments, the polypeptide is expressed more strongly when the bacterium is grown in vivo.

In some embodiments, the polypeptide is expressed more strongly when D. nodosus is grown in vitro in the presence of ovine hoof powder.

In some embodiments, the polypeptide is selected from the group consisting of PilT, PilU, ChpA, PilJ, PilI, PilG, PilH, Ppk, FimX, PilC, PilQ, RTX-like toxin (DNO_{—}0334), DNO_{—0335}-0336, putative large highly repetitive secreted protein (DNO_{—}0690), DNO_{—}0466, DNO_{—}0650, DNO_{—}1067, DNO_{—}0681, DNO_{—}0902, DNO_{—}0012 (SEQ ID NO: 179), DNO_{—}0033 (SEQ ID NO: 181), DNO_{—}0725 (SEQ ID NO: 187), and DNO_{—}1241 (SEQ ID NO: 190). In some embodiments, the polypeptide is selected from the group consisting of DNO_{—}0012 (SEQ ID NO: 179), DNO_{—}0033 (SEQ ID NO: 181), DNO_{—}0725 (SEQ ID NO: 187) and DNO_{—}1241 (SEQ ID NO: 190).

In some embodiments, the D. nodosus strain is one isolated from sheep, goats or cattle. In some embodiments the isolate is from sheep, and may be a virulent strain, for example strain VCS1703A serogroup G. Many other virulent strains are known in the art, for example those discussed herein.

The polypeptide may optionally be linked to a heterologous polypeptide. In one embodiment the heterologous polypeptide is an immunogenic carrier polypeptide. The linkage may be effected by chemical coupling, or the polypeptide of the invention may be expressed as a fusion protein with a heterologous polypeptide.

It will be clearly understood that the invention also encompasses biologically-active or immunogenic fragments, variants and derivatives of the polypeptides of the invention. In one embodiment the polypeptide of the invention is an outer membrane protein, and may comprise an extracellular domain. In another embodiment a fragment according to the invention corresponds to one or more extracellular domains of a cell surface polypeptide of D. nodosus. The fragment may comprise one or more epitopes.

In some embodiments, the polypeptide elicits an immune response which is protective against infection with D. nodosus. Preferably the polypeptide provides heterologous protection, i.e. protection against at least two different serotypes of D. nodosus. In some embodiments the polypeptide elicits antibodies which bind specifically to a D. nodosus polypeptide, and which may or may not be protective. Such antibodies are useful in immunoassays. In these embodiments the antibody may be a neutralizing antibody.

A polypeptide of the invention, or a biologically active or immunogenic fragment, derivative, or variant thereof, is useful in immunogenic compositions to prepare antisera or vaccines. A polypeptide of the invention, a fragment thereof such as a peptide, or a variant or derivative thereof, is also useful in assays to detect antibodies specific for the peptide, or for a polypeptide of which a portion has an amino acid sequence corresponding to an epitope within the peptide. These include diagnostic assays.

In a second aspect the invention provides a composition comprising a polypeptide according to the invention, or a biologically-active or immunogenic fragment, derivative, or variant thereof, together with a pharmaceutically or veterinarily acceptable carrier.

In a third aspect the invention provides an isolated antibody which specifically binds to a polypeptide of the invention, or to an epitope, fragment, derivative, or variant thereof. The antibodies of the invention may confer protection against infection with D. nodosus; such antibodies are useful in providing passive immunity to a subject to which the preparation is administered. Antibodies of the invention may react specifically with D. nodosus antigens, even though they do not confer protection against infection with D. nodosus; such antibodies are useful in immunoassays for detection of polypeptides of the invention or for diagnosis of D. nodosus infection.

The antibody may be raised in any convenient mammalian host, including but not limited to mice, rabbits, sheep, cattle, goats or horses, or may be produced in tissue or cell culture. The antibody may be polyclonal or monoclonal. Methods for production of polyclonal and monoclonal antibodies are very well known in the art.

In a fourth aspect the invention provides a composition comprising an antibody of the invention, together with a pharmaceutically or veterinarily acceptable carrier.

In a fifth aspect the invention provides an isolated nucleic acid molecule an isolated nucleic acid molecule which encodes a polypeptide of the invention, or a biologically active or immunogenic fragment, derivative, or variant thereof.

In a sixth aspect the invention provides an isolated nucleic acid molecule of the invention, together with a physiologically-acceptable carrier.

The invention further provides a vector comprising a nucleic acid molecule which encodes a polypeptide of the invention, or a biologically active or immunogenic fragment, derivative, or variant thereof, and a host cell comprising the vector. Preferably the vector is an expression cassette, and may comprise a preselected DNA sequence which is operably linked to a promoter which is functional in the host cell, in which the DNA sequence encodes one or more D. nodosus polypeptides of the invention. The host cell may be prokaryotic or eukaryotic in origin.

In a seventh aspect the invention provides a vaccine capable of treating or preventing a disease caused by D. nodosus, comprising one or more surface-exposed or secreted polypeptides of D. nodosus, in which the polypeptides comprise signal and lipopeptides and have fewer than 2 transmembrane domains, and in which the polypeptides are reactive against sera recovered from animals repeatedly infected with D. nodosus.

In some embodiments the polypeptides are encoded by genes located in gene loci selected from the group consisting of the loci listed in Table 3, Table 5 and Table 6.

In some embodiments the polypeptides are biologically active or immunogenic fragments, derivative, or variants of the surface-exposed or secreted proteins of D. nodosus, with the proviso that if individual polypeptides are used, then they are not AprV2 (DNO_{—}1167; SEQ ID NO: 189), AprV5 (DNO_{—}0603; SEQ ID NO: 183), BprV (DNO_{—}0605; SEQ ID NO: 184), a polypeptide encoded by the Omp1 locus, or a fimbrial polypeptide encoded by the fimA fimbrial subunit gene.

The polypeptides may be isolated from a natural source, produced recombinantly or synthetically produced, or may be extracted from D. nodosus bacteria or from D. nodosus culture supernatant or from biological material infected with D. nodosus.

In some embodiments the polypeptide is selected from the group consisting of:
a) a protein involved in transport of molecules across a D. nodosus outer membrane;
b) a protein involved in iron uptake;
c) a protein involved in transport of nutrients;
d) a protein involved in resistance of D. nodosus to heavy metals; and
e) a protein involved in synthesis or structural stability of the outer membrane.

In some embodiments, the polypeptide is expressed more strongly when the bacterium is grown in vivo.

In some embodiments, the polypeptide is expressed more strongly when D. nodosus is grown in vitro in the presence of ovine hoof powder.

In some embodiments the vaccine comprises
(a) one or more isolated immunogenic D. nodosus polypeptides according to the first aspect of the invention,
(b) one or more of YfeA (DNO_{—}0644; SEQ ID NO: 185), AprV2 (DNO_{—}1167; SEQ ID NO: 189), AprV5 (DNO_{—}0603; SEQ ID NO: 183), BprV (DNO_{—}0605; SEQ ID NO: 184), a polypeptide encoded by the Omp1 locus, and/or a fimbrial polypeptide encoded by the fimA fimbrial subunit gene, or
(c) an immunogenic fragment, derivative, or variant of (a) or (b),
   with the proviso that if the vaccine comprises AprV2 (DNO_{—}1167; SEQ ID NO: 189), AprV5 (DNO_{—}0603; SEQ ID NO: 183), BprV (DNO_{—}0605; SEQ ID NO: 184), a polypeptide encoded by the Omp1 locus, and/or a fimbrial polypeptide encoded by the fimA fimbrial subunit gene or an immunogenic fragment, derivative, or variant thereof, at least one of (a) is present.

The vaccine may additionally comprise an adjuvant. Preferably the vaccine provides heterologous protection, i.e. protection against at least two different serotypes of D. nodosus.

Two or more of the polypeptides may be administered either simultaneously or at different times, and may be administered to the same site or at different sites. It is contemplated that a mixture of two or more antigens will provide maximum cross-protection against different serogroups of D. nodosus.

In some embodiments, the subject is a sheep, and immunization results in an immune response which inhibits or prevents ovine footrot, or in the production of antibodies to the polypeptide employed as an immunogen. Both local and systemic administration is contemplated. Systemic administration of the vaccine is preferred.

While the invention is particularly directed to polypeptides suitable as antigens in vaccines for use in sheep, it will be clearly understood that it is applicable to any other animal which is susceptible to infection with D. nodosus, including but not limited to other animals with cloven hooves, such as members of the families Bovidae, including sheep, goats, cattle and antelope, and Cervidae, such as deer. It would be expected because of the close evolutionary relationship of these families of animals that a vaccine which was effective in sheep would also be effective in members of the other families. The invention is applicable to domestic, zoo or wild animals in all of these families. Many of the economically important animals in these families, such as goats, sheep and cattle, have very similar biology and share a high degree of genomic homology. The closeness of their relationship is illustrated by the fact that it is well known that some of these animals, such as goats and sheep, can interbreed. It would therefore be expected that a vaccine which was effective in sheep would also be effective in these other groups of animals. It will be appreciated that the diagnostic, therapeutic and prophylactic aspects of the invention are also applicable to subjects which have been exposed to an animal infected with D. nodosus, or to an environmental source contaminated with D. nodosus, such as pasture, soil, plant material or vehicles.

In an eighth aspect the invention provides a method of treating or preventing a disease or condition caused by D. nodosus, comprising the step of administering a polypeptide, antibody, nucleic acid and/or vaccine of the invention to a subject suffering from, suspected to be suffering from, or at risk of such a condition.

In a ninth aspect the invention provides a method of diagnosing a disease or condition caused by D. nodosus, comprising the step of detecting a polypeptide or fragment thereof, an antibody, and/or a nucleic acid molecule of the invention in a biological sample from a subject suffering from, suspected to be suffering from, or at risk of such a condition. Detection of the polypeptide or antibody may for example be achieved by a variety of methods, including but not limited to immunoassay methods such as radioimmunoassays, enzyme-linked immunosorbent assay (ELISA), chemiluminescence assays, scintillation proximity assays, immunohistochemistry, immunoblotting, for example Western blotting, and immunofluorescence. Detection of the nucleic acid molecule, or fragment, variant or derivative thereof may for example be achieved by nucleic acid amplification. Such methods are very well known in the art.

Thus the method of diagnosis may comprise contacting a sample of DNA obtained from a biological sample from a subject at risk of or suffering from D. nodosus infection with at least two oligonucleotides which bind to complementary strands of said DNA at preselected regions under conditions effective to amplify the DNA, so as to yield amplified DNA. The amplification may be effected by conventional methods, such as polymerase chain reaction. Alternatively the DNA may be obtained by reverse transcription of RNA from the cells. At least one of the oligonucleotides may be specific for DNA encoding a polypeptide of the invention. The presence of amplified DNA is then detected or determined by conventional methods. The presence of amplified DNA is indicative that the subject is infected with D. nodosus. It will be appreciated that any convenient biological sample from the subject may be used, including but not limited to blood and other biological fluids, sputum, hoof lesion exudate and tissue samples.

In a tenth aspect the invention provides the use of a polypeptide, antibody, nucleic acid and/or vaccine of the invention in the treatment or prevention of a disease or condition caused by D. nodosus.

In an eleventh aspect the invention provides the use of a polypeptide, antibody and/or nucleic acid molecule of the invention in the diagnosis of a disease or condition caused by D. nodosus. The disease or condition may be diagnosed following nucleic acid amplification.

The subject may be suspected of or at risk of having the disease or condition, or may have been, or may be suspected to have been, exposed to a subject known or suspected to be infected with D. nodosus.

In a twelfth aspect the invention provides a kit comprising one or more of a polypeptide, antibody, and/or nucleic acid molecule of the invention. The kit may be used as a diagnostic kit, and may comprise one or more pairs of nucleic acid molecules which can be used as primers for nucleic acid amplification.

In one embodiment the kit is a diagnostic kit for detecting the presence of DNA associated with D. nodosus in a sample, in which the kit comprises:

(a) a known amount of a first oligonucleotide which consists of at least about 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 to about 50 nucleotides, in which the oligonucleotide has at least about 70% contiguous sequence identity to a nucleic acid molecule of the invention;
(b) a known amount of a second oligonucleotide, in which the second oligonucleotide consists of at least about 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 to about 50 nucleotides, and in which the oligonucleotide has at least about 70% contiguous sequence identity to a nucleotide sequence which is complementary to a nucleic acid molecule of the invention; and optionally
(c) reagents for nucleic acid amplification.

Preferably the first oligonucleotide consists of at least about 12, 13, 14, 15, 16, 17, 18, 19, 20, 21, 22, 23, 24, 25, 26, 27, 28, 29, 30, 31, 32, 33, 34, 35, 36, 37, 38, or 39 to about 40 nucleotides, and even more preferably about 15, 16, 17, 18, 19, 20, 21, 22, 23, or 24 to about 25 nucleotides. Also preferably the second oligonucleotide consists of at least about 12, 13, 14, 15, 16, 17, 18, 19, 20, 21, 22, 23, 24, 25, 26, 27, 28, 29, 30, 31, 32, 33, 34, 35, 36, 37, 38, or 39 to about 40 nucleotides, and even more preferably about 15, 16, 17, 18, 19, 20, 21, 22, 23, or 24 to about 25 nucleotides. The skilled person will be able to optimize the oligonucleotides using methods known in the art.

In a thirteenth aspect, the invention provides a method for detecting a nucleic acid molecule encoding a polypeptide of D. nodosus, comprising the step of contacting a nucleic acid obtained from a biological sample from a subject with at least two oligonucleotides, under conditions effective to amplify the nucleic acid so as to yield an amount of amplified nucleic acid, in which the biological sample comprises cells suspected of containing a nucleic acid molecule encoding an immunogenic polypeptide, and at least one of the oligonucleotides is specific for a nucleic acid encoding a polypeptide of the invention, i.e. is able to hybridise to the nucleic acid under stringent conditions. Suitable conditions are well known in the art. The presence of the amplified nucleic acid may be detected by conventional methods, such as ethidium bromide or silver staining. A variety of other methods is known in the art.

In another aspect, the invention provides a method for detecting a nucleic acid molecule encoding a polypeptide of D. nodosus, comprising the step of contacting a nucleic acid obtained from a biological sample from a subject with at least two oligonucleotides, under conditions effective to amplify the nucleic acid so as to yield an amount of amplified nucleic acid, in which the biological sample comprises cells suspected of containing a nucleic acid molecule encoding an immunogenic polypeptide, and at least one of the oligonucleotides is specific for a nucleic acid encoding a polypeptide of the invention, i.e. is able to hybridise to the nucleic acid under stringent conditions. The presence of the amplified nucleic acid may be detected by conventional methods, such as ethidium bromide or silver staining. A variety of other methods is known in the art.

In a fourteenth aspect the invention provides a library of candidate immunogenic D. nodosus polypeptides, comprising the polypeptides encoded by the gene loci listed in Table 3, Table 5 or Table 6.

In a fifteenth aspect the invention provides a library of nucleic acid molecules encoding candidate immunogenic D. nodosus polypeptides, comprising nucleic acid molecules encoding the polypeptides encoded by the gene loci listed in Table 3, Table 5 or Table 6.

In a sixteenth aspect the invention provides a method of screening for candidate immunogenic D. nodosus polypeptides, comprising testing a polypeptide library according to the invention, or polypeptides expressed from the nucleic acid library according to the invention, for the ability to react with antibodies present in sera of animals previously exposed to D. nodosus infection or to elicit protective antibodies against D. nodosus.

Methods and pharmaceutical carriers for preparation of pharmaceutical compositions are well known in the art, as set out in textbooks such as Remington's Pharmaceutical Sciences, 20th Edition, Williams & Wilkins, Pennsylvania, USA.

The compounds and compositions of the invention may be administered by any suitable route, and the person skilled in the art will readily be able to determine the most suitable route and dose for the condition to be treated. Dosage will be at the discretion of the attendant physician or veterinarian, and will depend on the nature and state of the condition to be treated, the age and general state of health of the subject to be treated, the route of administration, and any previous treatment which may have been administered.

The carrier or diluent, and other excipients, will depend on the route of administration, and again the person skilled in the art will readily be able to determine the most suitable formulation for each particular case.

### BRIEF DESCRIPTION OF THE FIGURE

FIG. 1 shows immunoblots demonstrating recognition of D. nodosus proteins by pooled sera from experimentally infected sheep. A. Preimmune sera; B. Pooled immune sera; protein bands: 1, DNO_{—}0012 (SEQ ID NO: 179; 37 kDa+60 kDa NusA tag); 2, DNO_{—}0033 (SEQ ID NO: 181; 29 kDa); 3, DNO_{—}0603 (SEQ ID NO: 183; 67 kDa); 4, DNO_{—}0605 (SEQ ID NO: 184; 64 kDa); 5, DNO_{—}0644 (SEQ ID NO: 185; 34 kDa); 6, DNO_{—}0725 (SEQ ID NO: 187; 29 kDa); 7, DNO_{—}1167 (SEQ ID NO: 189; 66 kDa); 8, DNO_{—}1241 (SEQ ID NO: 190; 57 kDa) and 9, D. nodosus whole cell lysate.

### DETAILED DESCRIPTION

In an effort to identify novel D. nodosus immunogens to which sheep respond during natural infection, a detailed antigen profiling analysis was undertaken. We utilised a range of bioinformatics analyses of the unpublished annotated D. nodosus genome sequence, and our own unpublished experimental data in order to select genes according to their vaccine potential. This work was based on the premise that protective antigens are likely to be surface-exposed or secreted by the bacteria, and therefore accessible to the host immune system. These proteins are considered to be suitable candidate antigens. We used PSORTB (Gardy et al., 2003), SignalP (Neilsen et al, 1997) and ProteomeAnalyst (Neilsen et al, et al., 1997) to predict all outer membrane and secreted proteins and LipoP (Juncker et al., 2003) to predict all lipoproteins. Using these bioinformatics predictions we have identified 99 proteins which are likely to be surface-exposed or secreted by the bacteria. We also excluded proteins with >2 predicted transmembrane domains due to likely problems with purification. We have also identified 86 proteins which are differentially expressed in the presence of ovine hoof powder. Of the proteins tested so far, we have identified 8 proteins which react specifically with pooled antiserum from sheep infected with virulent D. nodosus, but not with control sera.

### DEFINITIONS

In the description of the invention and in the claims which follow, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

As used herein, the singular forms “a”, “an”, and “the” include the corresponding plural reference unless the context clearly dictates otherwise. Thus, for example, a reference to “an enzyme” includes a plurality of such enzymes, and a reference to “an amino acid” is a reference to one or more amino acids.

Where a range of values is expressed, it will be clearly understood that this range encompasses the upper and lower limits of the range, and all values in between these limits.

Unless defined otherwise, all technical and scientific terms used herein have the same meaning as commonly understood by one of ordinary skill in the art to which this invention belongs. Although any materials and methods similar or equivalent to those described herein can be used to practice or test the present invention, the preferred materials and methods are described.

The terms “isolated” and/or “purified” refer to an in vitro preparation of a molecule of the invention, or fragment, variant, or derivative thereof, so that the molecule is not substantially associated with molecules with which it normally occurs in vivo, and is substantially free of infectious agents.

The terms “polypeptide” and “protein” are herein used interchangeably. Where they are used to refer to an amino acid sequence of a naturally-occurring polypeptide, these terms are not meant to limit the amino acid sequence to the complete, native amino acid sequence associated with the recited polypeptide, but instead are meant also to encompass biologically active fragments, derivatives, or variants, including polypeptides having sequence similarity or sequence identity relative to the amino acid sequences provided herein. At various points in this specification individual polypeptides are identified by the name of the corresponding gene loci from which they are expressed.

As used herein, the term “surface polypeptide” means a polypeptide naturally located on the outer surface of D. nodosus, so that in vivo it is accessible to an immune response of a subject. An “antigenic” surface polypeptide is able to induce an antibody response. An “immunogenic” surface polypeptide is able to induce a specific immune response. Preferably the immunological response is an antibody response directed to a particular epitope on the surface polypeptide. In one embodiment the presence of antibodies specific for the polypeptide correlates with the D. nodosus infection status of the organism.

An epitope or antigenic determinant is a site on an antigen molecule which binds specifically to the antigen-combining site of an antibody or to a T cell receptor; it is a molecular structure which is recognized by an antibody. An epitope may be linear, e.g. a sequence of contiguous amino acid residues in a protein, or may be a three-dimensional, e.g. a part of the three-dimensional structure of a protein formed by non-contiguous amino acid residues. Antigens may be proteins, lipids or carbohydrates, and an antigen may comprise two or more different epitopes, and/or may have two or more repeated epitopes. Epitopes in any given polypeptide may readily be identified using well-known routine methods. A variety of algorithms may be used, for example for prediction of antigenicity (Hopp and Woods, 1983) or prediction of protein secondary structure (Chou and Fasman, 1974a, 1974b). Many others are known in the art; see for example www.epitopeinformatics.com. Commercial services are available for epitope analyses. For any given protein, epitopes may be empirically identified in a variety of ways, for example by testing proteolytically-generated fragments for their antigenic capacity, or preparing libraries of peptide fragments of the protein, either by expression on the filamentous phage PIII or PVIII surface proteins or by solid-phase peptide synthesis. See for example, Holzen et al. (2001) and references cited therein.

The term “vaccine antigen potential” means an estimate of the likely ability of a protein to elicit at least some degree of protective immunity. Any protein which confers some or total protection against a challenge with infective organisms can also be referred to as a potential vaccine candidate.

Polypeptides which have been subjected to chemical modifications, such as esterification, amidation, reduction, protection and the like, are referred to herein as “derivatives.” In particular, it is envisioned that a derivative of a polypeptide of the invention may have been modified in a manner that increases its stability in vivo, or which presents immunogenic epitopes in a more native configuration. Methods for preparing such derivatives are well known in the art. For example, a modification known to improve the immunogenicity, stability and/or bioavailability of peptides in vivo is the cyclization of the peptide, for example by formation of one or more disulphide bonds, as described in PCT/US98/25990 (WO99/29724). Another modification is the synthesis of a cyclic reverse sequence derivative (CRD) of a peptide of the invention, in which a linear peptide is synthesized with all D-form amino acids using the reverse (i.e. C-terminal to N-terminal) sequence of the peptide. The term “CRD” also includes cyclization by other mechanisms, e.g. via a peptidyl bond. Cyclized peptides with different kinds of linkages are known in the art; see EP 471,453 (amide bonds); EP 467,701 (disulphide bonds); EP 467,699 (thioether bonds). Other modifications are disclosed in Jameson et al. 1994; U.S. Pat. No. 4,992,463; and U.S. Pat. No. 5,091,396.

The surface polypeptides of the invention or fragments, variants or derivatives thereof can be prepared in vitro, e.g. by a solid phase peptide synthetic method or by a recombinant DNA approach.

As used herein, a “fusion protein” is a polypeptide comprising two or more polypeptides that have been joined together, for example after transcription and translation of two or more fused nucleic acid molecules. The two or more polypeptides may be the same or different. Thus the fusion protein may comprise two or more copies of a surface polypeptide of the invention, copies of more than one different surface polypeptide of the invention, or at least one surface polypeptide of the invention fused to any other polypeptide. In one embodiment of the invention the fusion protein comprises at least an immunogenic or antigenic portion of a plurality of D. nodosus outer membrane polypeptides.

If the surface polypeptide of the invention is expressed as a fusion protein, the fusion protein may be purified by methods specific for the surface polypeptide or non-surface polypeptide portion of the fusion polypeptide. For example, if the fusion polypeptide is a histidine-tagged fusion polypeptide, Ni-NTA resin may be employed to purify the fusion polypeptide. Epitope tags such as FLAG™ may also be used.

Fusion proteins can be prepared by in vitro transcription and translation reactions. An expression cassette can be employed to generate surface polypeptide gene-specific transcripts which are subsequently translated in vitro. The construction of vectors for use in in vitro transcription/translation reactions, and methods for such reactions, are well known in the art.

The polypeptides or fusion proteins of the invention may also be prepared by solid phase peptide synthesis, which is an established and widely used method, described in the following references: Stewart et al. 1969; Merrifield, 1963; Meienhofer in “Hormonal Proteins and Peptides,” ed.; C. H. Li, Vol. 2 (Academic Press, 1973), pp. 48-267; and Bavaay and Merrifield, “The Peptides,” eds. E. Gross and F. Meienhofer, Vol. 2 (Academic Press, 1980) pp. 3-285.

A “monoclonal antibody” is an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., 1975, or may be made by recombinant DNA methods (see, e.g., U.S. Pat. No. 4,816,567). The person skilled in the art will be aware of numerous other references, such as Goding, J. W. Monoclonal Antibodies: principles and practice (Academic Press, New York: 3^{rd} 1996).

“Nucleic acid molecule” as used herein refers to an oligonucleotide, polynucleotide, nucleotide, and fragments, variants, derivatives, and antisense molecules thereof, as well as to peptide nucleic acids (PNA), fragments, variants, derivatives and antisense molecules thereof, and to DNA or RNA of genomic or synthetic origin which can be single- or double-stranded, and represent the sense or antisense strand. Where “nucleic acid” is used to refer to a specific nucleic acid sequence, it is intended to encompass polynucleotides that encode a polypeptide that is functionally equivalent to the recited polypeptide, e.g., polynucleotides that are degenerate variants, or polynucleotides which encode biologically active fragments, variants, or derivatives of the polypeptide, including polynucleotides having substantial sequence similarity or sequence identity relative to the sequences provided herein.

The nucleic acid molecules of the invention, or fragments, variants, or derivatives thereof, may be used to prepare probes, primers or expression cassettes which, in turn, are useful to detect, amplify and express other outer membrane protein genes and related genes.

The terms “nucleotide sequence” and “nucleic acid sequence” are used herein interchangeably.

The term “antisense nucleic acid molecule” as used herein defines a sequence which is complementary to a nucleic acid molecule of interest or fragment, variant, or derivative thereof.

Nucleic acid molecules encoding a surface polypeptide of the invention, or a fragment, variant, or derivative thereof, may be obtained from any isolate of D. nodosus or from physiological fluid or tissue of animals infected with D. nodosus. Other sources of the DNA molecules of the invention include genomic libraries derived from any D. nodosus-infected eukaryotic cellular source. Moreover, DNA molecules which encode a subunit of a full-length surface polypeptide may be prepared in vitro, e.g. by synthesizing an oligonucleotide of about 200, preferably about 100, more preferably about 75, nucleotides in length, or by subcloning a portion of a DNA segment which encodes a particular OMP. A nucleic acid molecule encoding a surface polypeptide of the invention can be identified and isolated using standard methods, for example as described by Sambrook et al., Molecular Cloning. A Laboratory Manual, Cold Spring Harbor, N.Y. (1989).

A “variant” nucleic acid molecule of the invention is a nucleic acid molecule which has at least 80%, 81%, 82%, 83%, 84% or 85%, preferably at least about 90%, 91%, 92%, 93% or 94%, and more preferably at least about 95%, 96%, 97%, 98%, 99% but less than 100, contiguous nucleotide sequence homology or identity to the nucleotide sequence of the corresponding wild type nucleic acid molecule, which encodes a surface polypeptide of D. nodosus. A variant nucleic acid molecule of the invention may also include nucleotide bases not present in the corresponding wild type nucleic acid molecule, and/or internal deletions relative to the corresponding wild type nucleic acid molecule.

Nucleic acid molecules encoding amino acid sequence variants of surface polypeptides of the invention may be prepared by a variety of methods known in the art. These include, but are not limited to, isolation from a natural source (in the case of naturally-occurring amino acid sequence variants or serotypes) or preparation by site-directed mutagenesis, PCR mutagenesis, and cassette mutagenesis of an earlier prepared variant or a native surface polypeptide. An optimal oligonucleotide will have 12, 13, 14 or 15 nucleotides which are completely complementary to the template on either side of the nucleotide(s) coding for the mutation. This ensures that the oligonucleotide will hybridize properly to the single-stranded DNA template molecule. The oligonucleotides are readily synthesized using techniques known in the art, such as that described by Crea et al., 1978.

“Cloning” of DNA, also known as gene cloning or molecular cloning, refers to the use of recombinant DNA technology to insert a desired DNA fragment into a cloning vector, which is then introduced into a host cell in which it can replicate, and culturing the host cell. The DNA may be isolated from its natural source, or may be cDNA or synthetic DNA.

A “vector” or “cloning vector” is a DNA molecule originating from a virus, a plasmid, or the cell of a higher organism into which another DNA fragment of appropriate size can be integrated without loss of the vector's capacity for self-replication; vectors are routinely used to introduce foreign DNA into host cells, in which the DNA can be reproduced in large quantities. Examples include plasmids, cosmids, and yeast artificial chromosomes; vectors are often recombinant molecules containing DNA sequences from several sources.

A vector may include one or more nucleic acid sequences, such as an origin of replication, which permit the vector to replicate in a host cell. A vector also may include one or more selectable marker genes and other genetic elements known in the art. The term “vector” as used herein is intended to encompass any carrier for nucleic acid, including plasmids, cosmids and phage. Preferably the vector is an expression cassette.

To prepare expression cassettes for transformation of host organisms, a recombinant or preselected nucleic acid sequence or segment may be circular or linear, double-stranded or single-stranded. A preselected DNA sequence which encodes an RNA sequence which is substantially complementary to a RNA sequence encoding surface polypeptide is typically a “sense” DNA sequence cloned into a cassette in the opposite orientation (i.e. 3′ to 5′ rather than 5′ to 3′). Generally the preselected DNA sequence or segment is in the form of chimeric DNA, such as plasmid DNA, which can also contain coding regions flanked by control sequences which promote the expression of the preselected DNA present in the resultant cell line.

“Chimeric” means that a vector comprises DNA from at least two different species, or comprises DNA from the same species, which is linked or associated in a manner which does not occur in the “native” or wild type of the species.

Apart from preselected DNA sequences which serve as transcription units for surface polypeptides, or fragments, variants, or derivatives thereof, a portion of the preselected DNA may be untranscribed, serving a regulatory or a structural function. For example, the preselected DNA may itself comprise a promoter which is active in the host cell, or may utilize a promoter already present in the genome that is the transformation target. Many promoter elements well known to the art may be employed in the practice of the invention.

“Control sequences” are nucleic acid sequences necessary for the expression of an operably-linked coding sequence in a particular host organism. Control sequences which are suitable for prokaryotic cells, for example, include a promoter, and optionally an operator sequence, and a ribosome binding site. Such elements may or may not be necessary for the function of the nucleic acid molecule, but may provide improved expression of the nucleic acid molecule by affecting factors such as transcription or stability of mRNA, and may be included in the nucleic acid molecule to obtain optimal performance of the transforming DNA in the cell.

“Operably linked” means that a nucleic acid molecule is placed in a functional relationship with another nucleic acid molecule. Generally, “Operationally linked” means that the nucleic acid molecules being linked are contiguous and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The preselected nucleic acid molecule to be introduced into the cells further will generally contain a selectable marker gene and/or a reporter gene, to facilitate identification and selection of transformed cells from the population of cells sought to be transformed. Alternatively, the selectable marker may be carried on a separate piece of DNA and used in a co-transformation procedure. Both selectable markers and reporter genes may be flanked with appropriate regulatory sequences to enable expression in the host cells. Useful selectable markers are well known in the art, and include antibiotic genes, such as those listed in Table 1 of U.S. Pat. No. 5,848,956 by Lundquist et al.

Reporter genes are used for identifying potentially transformed cells and for evaluating the functionality of regulatory sequences. Reporter genes which encode for easily assayable proteins are well known in the art. In general, a reporter gene is a gene which is not present in or expressed by the recipient organism or tissue and which encodes a protein whose expression is manifested by some easily detectable property, e.g. enzymatic activity. Preferred genes include the lacZ encoding β-galactosidase and the chloramphenicol acetyl transferase gene (cat) from Tn9 of E. coli, the β-glucuronidase gene (gus) of the uidA locus of E. coli, and the luciferase gene from the firefly Photinus. Expression of the reporter gene is assayed at a suitable time after a nucleic acid molecule has been introduced into the recipient cells.

As used herein, the term “recombinant” nucleic acid molecule refers to a nucleic acid molecule which has been derived or isolated from any appropriate cellular source, and which may subsequently be chemically altered in vitro, so that its sequence is not naturally occurring, or corresponds to naturally occurring sequences which are not positioned as they would be positioned in a genome which has not been transformed with exogenous DNA. A recombinant nucleic acid “derived” from a source may be a DNA sequence which is identified as a useful fragment within a given organism, and which is then chemically synthesized in essentially pure form. Therefore the term “recombinant nucleic acid” includes completely synthetic DNA sequences, semi-synthetic DNA sequences, DNA sequences isolated from biological sources, and DNA sequences derived from RNA, and mixtures thereof. The term “derived” with respect to a RNA molecule means that the RNA molecule has complementary sequence identity to a particular DNA molecule. A DNA “isolated” from a source may be a DNA sequence which is excised or removed from the source by chemical means, e.g. by the use of restriction endonucleases, so that it can be further manipulated for use in the invention, by genetic engineering methodology.

As used herein, the term “host cell” is intended to refer to well-characterized homogenous, biologically pure populations of cells. The cell line or host cell is preferably of bacterial origin, and most conveniently is Escherichia coli. “Transfected” or “transformed” is used herein to include any host cell or cell line, the genome of which has been altered or augmented by the presence of at least one preselected DNA sequence, which DNA is also referred to in the art of genetic engineering as “heterologous DNA” “recombinant DNA”, “exogenous DNA”, “genetically engineered”, “non-native” or “foreign DNA”, in which the DNA was isolated and introduced into the genome of the host cell or cell line by the process of genetic engineering. The host cells are typically produced by transfection with a DNA sequence in a plasmid expression vector, a viral expression vector, or as an isolated linear DNA sequence. Preferably the transfected DNA is a chromosomally-integrated recombinant DNA sequence, which comprises a gene encoding an OMP or its complement, which host cell may or may not express significant levels of autologous or “native” OMP.

General methods for constructing recombinant DNA which can transform target cells are well known to those skilled in the art, and the same compositions and methods of construction may be utilized to produce the DNA useful herein. For example, J. Sambrook et al., Molecular Cloning. A Laboratory Manual, Cold Spring Harbor Laboratory Press (2d ed., 1989), provides suitable methods of recombinant DNA construction.

The recombinant DNA can be readily introduced into the host cells by transfection with an expression vector comprising DNA encoding an OMP or its complement, by any procedure useful for the introduction into a particular cell, e.g., physical or biological methods (e.g., recombinant phage or viruses), to yield a transformed cell having the recombinant DNA stably integrated into its genome, so that the DNA molecules, sequences, or segments, of the present invention are expressed by the host cell.

Physical methods for introducing a recombinant nucleic acid molecule into a host cell include calcium phosphate precipitation, lipofection, particle bombardment, microinjection, electroporation, and the like. The main advantage of physical methods is that they are not associated with pathological or oncogenic processes of viruses in eukaryotic hosts. However, they are less precise, often resulting in multiple copy insertions, random integration, disruption of foreign and endogenous gene sequences, and unpredictable expression.

To confirm the presence of the preselected DNA sequence in the host cell, a variety of assays may be performed. Such assays include molecular biological assays well known to those of skill in the art, such as Southern and Northern blotting, RT-PCR and PCR; biochemical assays, such as detecting the presence or absence of a particular OMP, e.g. by immunological means such as ELISA assays and Western blots, or by functional assays to identify specific proteins falling within the scope of the invention.

To detect and quantify RNA produced from introduced preselected DNA segments, reverse transcription PCR (RT-PCR) may be employed. In this application of PCR, it is first necessary to reverse transcribe RNA into DNA, using enzymes such as reverse transcriptase, and then through the use of conventional PCR techniques amplify the DNA. In most instances PCR techniques will not demonstrate integrity of the RNA product, but Northern blotting demonstrates the presence of an RNA species, and gives information about the integrity of that RNA. The presence or absence of an RNA species can also be determined using dot or slot blot Northern hybridizations. These techniques are modifications of Northern blotting, and only demonstrate the presence or absence of an RNA species.

While Southern blotting and PCR may be used to detect the preselected DNA segment in question, they do not provide information as to whether the preselected DNA segment is being expressed. Expression may be evaluated by specifically identifying the products of the introduced preselected DNA sequences or evaluating the phenotypic changes brought about by the expression of the introduced preselected DNA segment in the host cell.

Recovery or isolation of a given fragment of DNA from a restriction digest employs methods well known in the art, such as separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. See Lawn et al., 1981, and Goeddel et al., 1980.

“Polymerase chain reaction” (PCR) refers to a procedure in which amounts of a preselected fragment of nucleic acid, RNA and/or DNA, are amplified, such as described in U.S. Pat. No. 4,683,195. Sequence information from the ends of the region of interest or beyond is generally employed to design oligonucleotide primers comprising at least 7-8 nucleotides. These primers will be identical or similar in sequence to opposite strands of the template to be amplified. PCR can be used to amplify specific RNA sequences, specific DNA sequences from total genomic DNA, and cDNA transcribed from total cellular RNA, bacteriophage or plasmid sequences, and the like. See generally Mullis et al., Cold Spring Harbor Symp. Quant. Biol., 51, 263 (1987); Erlich, ed., PCR Technology, (Stockton Press, New York, 1989). Thus PCR-based cloning approaches rely upon conserved sequences deduced from alignments of related gene or polypeptide sequences.

Primer oligonucleotide sequences are synthesized so as to correspond to highly-conserved regions of proteins or nucleotide sequences which were identified and compared to generate the primers, e.g., by a sequence comparison of other bacterial OMP genes. One primer is predicted to anneal to the antisense strand, and another primer is predicted to anneal to the sense strand, of a DNA molecule which encodes a specific protein. The products of each PCR reaction are separated, for example by agarose gel electrophoresis, and all consistently amplified products are gel-purified and cloned directly into a suitable vector, such as a known plasmid vector. The resultant plasmids are subjected to restriction endonuclease and dideoxy sequencing of double-stranded plasmid DNAs. Alternatively the gel-purified fragment can be directly sequenced.

“Stringent conditions” for hybridization or annealing of nucleic acid molecules are well known in the art, and include those which

(1) employ low ionic strength and high temperature for washing, for example 0.015 M NaCl/0.0015 M sodium citrate/0.1% sodium dodecyl sulfate (SDS) at 50° C., or
(2) employ during hybridization a denaturing agent such as formamide, for example 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is use of 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/mL), 0.1% SDS, and 10% dextran sulfate at 42° C., with washes at 42° C. in 0.2×SSC and 0.1% SDS. See Maniatis et al, op. cit.

Generally, the terms “treating”, “treatment” and the like are used herein to mean affecting a subject, tissue or cell to obtain a desired pharmacological and/or physiological effect. The effect may be prophylactic in terms of completely or partially preventing a disease or condition, or sign or symptom thereof, and/or may be therapeutic in terms of a partial or complete cure of a disease or condition. “Treating” as used herein covers any treatment of, or prevention of, disease in the subject, and includes preventing the disease from occurring in a subject who may be predisposed to the disease, but has not yet been diagnosed as having it; inhibiting the disease, i.e., arresting its development; or relieving or ameliorating the effects of the disease, i.e., cause regression of the effects of the disease. As used herein “condition” means abnormal functioning, and may be any condition caused by D. nodosus.

As used herein “diagnosis” means identifying the nature or cause of a disease or condition, such as the diagnosis of D. nodosus infection.

The invention further relates to diagnostic assays for use in veterinary medicine. For diagnosis of D. nodosus infection status, the presence of antibodies to a D. nodosus polypeptide of the invention in animal serum or in exudate from footrot lesions is determined. Many types of test formats may be used. Such tests include, but are not limited to, immunofluorescence assay, radioimmunoassy, radioimmunosorbent test, enzyme-linked immunosorbent assay, scintillation proximity assays, immunohistochemistry, immunoblotting, for example Western blotting, immunofluorescence, agglutination and hemagglutination.

Alternatively diagnosis may involve PCR or DNA hybridization detection of a specific gene encoding a D. nodosus polypeptide of the invention. The diagnostic assays can be performed using standard protocols.

For example, a diagnostic assay of the invention can be constructed by coating all or a unique portion of a polypeptide or peptide, or an isolated D. nodosus preparation (the antigen) on a solid support, for example a plastic bead, a test tube, a fibre strip, a microtitration plate or a membrane, and contacting it with a sample of serum, lesion exudate or other physiological fluid taken from a subject suspected of having a D. nodosus infection. Following removal of the sample, any antibody bound to the immobilized antigen can be detected, preferably by reacting the binary antibody-antigen complexes with a “detection antibody”, which comprises a detectable label or a binding site for a detectable label. Suitable detectable labels are enzymes, fluorescent labels or radiolabels. Binding sites for detectable labels include avidin, biotin, streptavidin and the like. In another embodiment of the diagnostic assay of the invention, all or a unique portion of the antigen is bound to an inert particle, for example a bentonite, polystyrene, or latex particle. The particles are mixed with serum from a subject in a well of a plastic agglutination tray. The presence or absence of antibodies in the subject's serum is determined by observing the settling pattern of the particles in the well.

Isolated surface polypeptides or nucleic acids, of the invention may be administered to a subject in an amount effective to elicit an immune response specific for D. nodosus. Methods and pharmaceutical carriers for preparation of pharmaceutical and veterinary compositions are well known in the art, as set out in textbooks such as Remington's Pharmaceutical Sciences, 20th Edition, Williams & Wilkins, Pennsylvania, USA. The compounds and compositions of the invention may be administered by any suitable route, and the person skilled in the art will readily be able to determine the most suitable route and dose for the condition to be treated. Dosage will be at the discretion of the attendant veterinarian, and will depend on the nature and state of the disease or condition to be treated, the age and general state of health of the subject to be treated, the route of administration, and any previous treatment which may have been administered.

Typically, dosages used in vitro may provide useful guidance in the amounts useful for in situ administration of the pharmaceutical composition, and animal models may be used to determine effective dosages for treatment of the cytotoxic side effects. Various considerations are described in references such as Langer, 1990.

The carrier or diluent, and other excipients, will depend on the route of administration, and again the person skilled in the art will readily be able to determine the most suitable formulation and dosage for each particular case.

In general, the dosage of recombinant bacteria required for efficacy will range from about 10⁴, 10⁹, 10⁶, 10⁷, 10⁸, 10⁹, 10¹⁰ or 10¹¹ to 10¹², preferably about 10⁹, 10⁶, 10⁷, 10⁸ or 10⁹ to 10¹⁰, and more preferably about 10⁹, 10⁷ or 10⁸ to 10⁹, colony-forming units (CFU), although other amounts may prove efficacious. For proteins and peptides of the invention, the dosage required is about 1 pg to about 10 mg, preferably about 10 pg to about 1 mg, and more preferably about 100 pg to about 500 μg, although other dosages may be employed. In particular, for administration of a protein or peptide of the invention to a sheep, the amount administered may be at dosages of at least about 1 pg to about 10 mg, preferably about 10 μg to about 1 mg, and more preferably about 100, 200, 300, 400 or 500 μg, although other dosages may provide beneficial results. Dosages within these ranges can be administered via bolus doses or via a plurality of unit dosage forms, until the desired effects have been obtained.

The amount administered will vary depending on various factors, including, but not limited to, the specific immunogen chosen, the weight, physical condition and age of the subject, and the route of inoculation. Thus for polypeptide and peptides, the absolute weight of polypeptide or peptide in a given unit dosage form of vaccine can vary widely, and depends upon factors such as the species, age, weight and physical condition of the subject to be vaccinated, as well as the method of administration. Such factors can be readily determined by the veterinarian employing animal models or other test systems which are well known to the art.

Methods for assessing the efficacy of the vaccine are known in the art. For example, the ability of the vaccine to prevent the development of footrot lesions or to reduce the severity of such lesions following challenge with D. nodosus is assessed. Suitable grading systems are known; see for example the review by Whittington and Nicholls (1995). In some embodiments the “total weighted footscore” system described therein is used.

A unit dose of a protein or peptide vaccine is preferably administered parenterally, e.g. by subcutaneous or intramuscular injection.

The proteins or peptides of the invention may also be conjugated or linked to an immunogenic protein, such as keyhole limpet haemocyanin (KLH) or albumin, to enhance their immunogenicity. For example, synthetic peptides are coupled to KLH through the C-terminal cysteine of the peptide using the heterobifunctional reagent N-γ-maleimidobutyric acid N-hydroxysuccinimide ester (GMBS; Sigma) according to the manufacturer's directions. The carrier-conjugated peptides are stored at −20° C. until used.

The compositions of recombinant bacteria which express the polypeptides of the invention may be administered as live, modified-live (attenuated) or inactivated bacteria, or as a combination of attenuated, inactivated, and/or live bacteria, or in combination with a protein or peptide of the invention, or any combination thereof. The bacteria may be inactivated by agents including, but not limited to, formalin, phenol, ultraviolet radiation, and β-propiolactone.

Immunogenic compositions are typically prepared for injection or infusion, either as liquid solutions or suspensions. Solid forms suitable for solution in, or suspension in, liquid vehicles prior to injection or infusion may also be prepared. The preparation may also be emulsified. The active ingredient may be mixed with diluents, carriers or excipients which are physiologically acceptable and compatible with the active ingredient(s). Suitable carriers can be positively or negatively-charged or neutral pyridine-containing liposomes, oil emulsions, such as live-in-oil; killed-in-oil, or water-in-oil emulsions; aluminium hydroxide; oil emulsion with terpene oils or squalene; or aqueous. Suitable diluents and excipients include water, saline, PBS, glycerol, or the like, and combinations thereof. In addition, if desired, the compositions may contain minor amounts of auxiliary substances such as wetting or emulsifying agents, stabilizing or pH-buffering agents, and the like.

Aqueous suspensions normally contain the active materials in admixture with excipients suitable for the manufacture of aqueous suspensions. Such excipients may be suspending agents such as sodium carboxymethyl cellulose, methyl cellulose, hydroxypropylmethylcellulose, sodium alginate, polyvinylpyrrolidone, gum tragacanth and gum acacia; dispersing or wetting agents, which may be

(a) naturally occurring phosphatide such as lecithin;
(b) a condensation product of an alkylene oxide with a fatty acid, for example, polyoxyethylene stearate;
(c) a condensation product of ethylene oxide with a long chain aliphatic alcohol, for example, heptadecaethylenoxycetanol;
(d) a condensation product of ethylene oxide with a partial ester derived from a fatty acid and hexitol such as polyoxyethylene sorbitol monooleate, or
(e) a condensation product of ethylene oxide with a partial ester derived from fatty acids and hexitol anhydrides, for example polyoxyethylene sorbitan monooleate.

The compositions may be in the form of a sterile injectable aqueous or oleaginous suspension. This suspension may be formulated according to known methods using suitable dispersing or wetting agents and suspending agents such as those mentioned above. The sterile injectable preparation may also be a sterile injectable solution or suspension in a non-toxic parenterally-acceptable diluent or solvent, for example, as a solution in 1,3-butanediol. Among the acceptable vehicles and solvents which may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid may be used in the preparation of injectables.

To prepare a vaccine composition comprising a surface polypeptide, the polypeptide can be isolated as described above, lyophilized and stabilized. Alternatively the surface polypeptide may be modified so as to produce a derivative, as described above. The polypeptide antigen may then be adjusted to an appropriate concentration, optionally combined with a suitable carrier and/or suitable vaccine adjuvant, and preferably packaged for use as a vaccine.

An “adjuvant” is a substance which augments, stimulates, activates, potentiates, or modulates the immune response at either the cellular or humoral level. An adjuvant may be added to a vaccine, or may be administered before administering an antigen, in order to improve the immune response, so that less vaccine is needed to produce the immune response. Widely-used adjuvants include alum, ISCOMs which comprise saponins such as Quil A, liposomes, and agents such as Freund's adjuvant, Bacillus Calmette Guerin (BCG), Corynebacterium parvum or mycobacterial peptides which contain bacterial antigens. Other adjuvants include, but are not limited to, surfactants, e.g. hexadecylamine, octadecylamine, lysolecithin, di-methyldioctadecylammonium bromide, N,N-dioctadecyl-n′-N-bis(2-hydroxyethylpropane diamine), methoxyhexadecyl-glycerol, and pluronic polyols; polyanions, e.g. pyran, dextran sulphate, poly IC, polyacrylic acid, and carbopol; peptides, e.g. muramyl dipeptide, dimethylglycine, and tuftsin; oil emulsions, and mixtures thereof. Only some of these are currently approved for human or veterinary use; others are in clinical trial. The immunogenic product may be incorporated into liposomes for use in a vaccine formulation, or may be conjugated to polysaccharides or other polymers. Some adjuvants are endogenous to the subject to be vaccinated; these include histamine, interferon, transfer factor, tuftsin and interleukin-1. Their mode of action is either non-specific, resulting in increased immune responsiveness to a wide variety of antigens, or antigen-specific, i.e. affecting a restricted type of immune response to a narrow group of antigens.

It is to be clearly understood that this invention is not limited to the particular materials and methods described herein, as these may vary. It is also to be understood that the terminology used herein is for the purpose of describing particular embodiments only, and it is not intended to limit the scope of the present invention, which will be limited only by the appended claims.

Unless otherwise indicated, the present invention employs conventional chemistry, protein chemistry, molecular biological and enzymological techniques within the capacity of those skilled in the art. Such techniques are well known to the skilled worker, and are explained fully in the literature. See Coligan, Dunn, Ploegh, Speicher and Wingfield: “Current protocols in Protein Science” (1999) Volumes I and II (John Wiley & Sons Inc.); Sambrook, Fritsch and Maniatis: “Molecular Cloning: A Laboratory Manual” (2001); Shuler, M. L.: Bioprocess Engineering: Basic Concepts (2nd Edition, Prentice-Hall International, 1991); Glazer, A. N., DeLange, R. J., and Sigman, D. S.: Chemical Modification of Proteins (North Holland Publishing Company, Amsterdam, 1975); Graves, D. J., Martin, B. L., and Wang, J. H.: Co- and post-translational modification of proteins: chemical principles and biological effects (Oxford University Press, 1994); Lundblad, R. L. (1995) Techniques in protein modification. CRC Press, Inc. Boca Raton, Fl. USA; and Goding, J. W Monoclonal Antibodies: principles and practice (Academic Press, New York: 3^{rd} ed. 1996).

### Abbreviations Used Herein are as Follows:

CDS predicted coding sequences
ELISA enzyme-linked immunosorbent assay
EYE Eugon yeast extract
LPS lipopolysaccharide
3-mer trinucleotide
OMP outer membrane proteins
ORF open reading frame
PBS phosphate-buffered saline
QRT-PCR quantitative real-time polymerase chain reaction
SDS-PAGE sodium dodecyl sulphate polyacrylamide gel electrophoresis

The surface of Gram-negative bacteria is critical for the interaction of the bacterium with the environment. It consists primarily of lipopolysaccharide (LPS), phospholipids and proteins. The LPS and phospholipids provide a significant permeability barrier to hydrophobic compounds, while the proteins known as outer membrane proteins (OMPs) are generally involved in outer membrane stability and in transport of various molecules in and out of the cell. These OMPs include porins, which allow non-specific diffusion of charged and neutral solutes, and high affinity transporters, which mediate transport of specific ligands in and out of the cell. The expression of the various OMPs is influenced by the extracellular environment. While these principles apply to most Gram-negative bacteria, the surface of D. nodosus has not hitherto been well-characterized, and consequently it was not known prior to the present study whether this organism fitted the typical pattern.

The D. nodosus strain VCS1703A was chosen for whole genome sequence analysis, as it is both virulent and naturally transformable, and has been used in footrot virulence studies (Kennan et al, 2001). The genome data were used for the identification of vaccine candidates by screening heterologously-expressed predicted coding sequences (CDSs) with sheep immune sera. These investigations have led to the identification of novel surface or secreted proteins with substantive vaccine potential. The effectiveness of this approach, which has been termed “reverse vaccinology” or “reverse immunology” has been reported for several organisms, including Plasmodium falciparum (Haddad et al., 2004), Streptococcus pneumoniae (Wizemann et al., 2001), Treponema pallidum (McKevitt et al., 2003), Neisseria meningitidis serogroup B (Pizza et al., 2000), and Chlamydia pneumoniae (Montigiani et al., 2002). However, although this approach appears to be applicable to a wide range of organisms, the individual antigens which confer protection in any individual case cannot be predicted.

This approach to immunogen discovery suffers from the drawbacks that
(a) it is limited to protein coding regions which can be expressed in E. coli or Ps. Aeruginosa or other cells suitable for heterologous protein expression;
(b) antibodies specific for conformational epitopes which are denatured in SDS-PAGE will not be detected;
(c) a lack of antibody recognition to a given recombinant protein may be the result either of a poor immune response to the antigen or of a reflection of its low level or total absence of expression in the host.

In the present case, alternative strains of E. coli are available which are suitable as expression hosts, and Ps aerginosa may also be used. Conformational epitopes may be identified by testing the vaccine potential of soluble non-denatured antigens in vivo, for examples in sheep.

The invention will now be described in detail by way of reference only to the following non-limiting examples and drawings.

The D. nodosus strain VCS1703A was chosen for whole genome sequence analysis, as it is both virulent and naturally transformable, and has been used in footrot virulence studies (Kennan et al, 2001). The genome data was used for the identification of vaccine candidates by screening heterologously-expressed predicted coding sequences (CDSs) with sheep immune sera. These investigations have led to the identification of novel surface or secreted proteins with substantive vaccine potential.

### Materials and Methods

### Growth Conditions

D. nodosus strains were grown either on Eugon (Difco) yeast extract (EYE) agar containing 5% defibrinated horse blood (Equicell) or in Eugon (Difco) broth with yeast extract in an anaerobic chamber, as previously described (Parker et al, 2005). Anaerobic jar experiments were performed using AnaeroGen and CampyGen atmosphere generation sachets (Oxoid) in Oxoid anaerobic jars.

### Example 1

### Genome Sequencing and Annotation

The complete genome sequence of the virulent serogroup G D. nodosus strain VCS1703A was determined using the whole-genome shotgun method as previously described (Fraser, 1997). Physical and sequencing gaps were closed using a combination of primer walking, generation and sequencing of transposon-tagged libraries of large-insert clones, and multiplex PCR (Tettelin, 1998). Identification of putative protein-encoding genes and annotation of the genome were performed as previously described (Bulach, 2006). An initial set of genes predicted to encode proteins was identified with GLIMMER (Delcher, 2002). Genes consisting of fewer than 30 codons and those containing overlaps were eliminated. Frame shifts and point mutations were corrected or designated “authentic”.

Functional assignment, identification of membrane-spanning domains, determination of paralogous gene families and identification of regions of unusual nucleotide composition, sequence alignments and phylogenetic trees were performed as previously described (Fraser, 1997).

The complete annotated genome sequence is available at GenBank accession number CP000513, and is incorporated herein by this reference. This sequence was published on 4^{th} May 2007. The sequences of the polypeptides listed herein, including those in Table 5, can be downloaded from GenBank accession No. CP000513, or can readily be derived from this source.

### Example 2

### Virulence Factors and Strain Diversity

D. nodosus differs from other organisms with small genomes in its surprising extent of apparent lateral gene transfer and strain diversity. This was assessed by analysis of regions of atypical trinucleotide composition, combined with comparative genomic microarray experiments on eight selected D. nodosus isolates using a custom 70mer oligonucleotide array for all 1299 genes derived from the whole genome sequence. The strains used are summarized in Table 1.

TABLE 1
Characteristics of strains used in comparative
genomic hybridization analysis

| ORIGINAL | | | SERO- | |
|---|---|---|---|---|
| No | VIRULENCE | TYPE | GROUP | SOURCE |
| AC424 | BENIGN | II | H | Albany, WA, Australia |
| VCS1703A | VIRULENT | I | G | University of Sydney, |
| | | | | Australia |
| VCS1690A | VIRULENT | II | H | University of Sydney, |
| | | | | Australia |
| A198 | VIRULENT | I | A | University of Sydney, |
| | | | | Australia |
| AC390 | BENIGN | II | D | Albany, WA, Australia |
| 806(2a) | INTER- | I | I | Wagga Wagga, NSW, |
| | MEDIATE | | | Australia |
| CS101 | BENIGN | I | G | CSIRO, Vic, Australia |
| 375(4b) | — | — | — | Wagga Wagga, NSW, |
| | | | | Australia |
| HA320 | INTER- | — | — | Hamilton, Vic, |
| | MEDIATE | | | Australia |

Genomic DNA was extracted from two-day-old EYE agar cultures of D. nodosus using the DNeasy extraction kit (Qiagen). DNA (4 μg) was digested with 20 U of AluI for 2 h at 37° C. and purified (PCR purification kit, Qiagen). Labelled genomic DNA (2 μg in 20 μl) was prepared by the addition of 25 μg of random hexamers with 20 μl of reaction buffer (42 mM 2-mercaptoethanol, 21 mM MgCl₂, 210 mM Tris-HCl pH7.0), boiled for 5 min then placed on ice. Fluorescent dyes (60 nM, Cy5-dUTP or Cy3-dUTP, Amersham Biosciences) were coupled using 40 U of Klenow enzyme and nucleotides (1.2 mM dCTP, dGTP, dATP and 0.6 mM dTTP) in a final volume of 50 μl. Reactions were incubated for two hours and stopped in the presence of 50 mM EDTA, pH 8.0. Reaction mixtures were purified with Microcon columns (Millipore) and concentrated in a Speedvac SVC (Savant). Samples were then hybridised, washed, scanned and analysed as previously described (Parker, 2006).

The D. nodosus VCS1703A genome was compared to other genomes at the nucleotide level by suffix tree analysis using MUMmer. D. nodosus CDSs were compared by BLAST against the complete set of non-redundant CDSs, using an E-value cut-off of 1×10⁻⁵. Genes which had greater than a 1.5-fold change in signal and a Wilcoxon signed ranked p-value<0.05 were included in the final data set.

Microarray data have been deposited at the National Center for Biotechnology Information's Gene Expression Omnibus (GEO) database (http://www.ncbi.nlm.nih.gov/geo) under the series accession number GSE5157, and are incorporated herein by this reference.

Twenty-one distinct zones of unusual trinucleotide composition were identified; many of these regions also corresponded to strain-specific regions of variability identified by microarray analysis.

The largest region of atypical trinucleotide composition is a 38.4 kb integrated Mu-like bacteriophage. This region encodes homologues of phage terminase and head and tail morphogenesis proteins. This region is divergent in at least five of the eight isolates examined (confirmed by Southern blotting, data not shown). attP-like binding sites and phage-like integrases have been previously reported to be associated with D. nodosus genomic islands (Haring et al, 1995); however, the sequence disclosed in this specification is the first complete D. nodosus bacteriophage-like sequence reported.

The distribution of all 64 trinucleotides (3-mers) was determined, and the 3-mer distribution in 2,000-bp windows which overlapped by half their length (1,000 bp) across the genome was computed. For each window, we computed the χ² statistic on the difference between its 3-mer content and that of the whole chromosome. A large value for χ² indicates the 3-mer composition in this window is different from the rest of the chromosome. Probability values for this analysis are based on assumptions that the DNA composition is relatively uniform throughout the genome, and that 3-mer composition is independent. Because these assumptions may be incorrect, we prefer to interpret high χ² values as indicators of regions on the chromosome which appear unusual and demand further scrutiny.

Two vap loci which include phage-like integrases and plasmid gene homologues are present in D. nodosus VCS1703A, corresponding to the regions of unusual trinucleotide composition. These two vap loci are conserved in only two of the eight D. nodosus isolates (HA320, with intermediate virulence; and the virulent strain A198), with the remainder of the isolates exhibiting significant variability within these loci. Similarly, strain VCS1703A has the vr1 island, also associated with significant unusual trinucleotide composition. The vr1 island from strain VCS1703A has only 3 by differences to the 27 kb vrl region from strain A198. The vrl region was also present in strain HA320 and AC424, a benign isolate. Despite the association of the vap and vrl loci with D. nodosus virulence, none of the genes within these loci has yet been assigned a known virulence function.

Type IV fimbriae are present on many important pathogens, such as Pseudomonas aeruginosa, Neisseria sp, Legionella pneumophila, and enteropathogenic Escherichia coli, and are required for virulence, natural transformation, adhesion, twitching motility and protease secretion in D. nodosus (Parker et al, 2006; Kennan, et al 2001).

The type IV fimbrial biogenesis genes are scattered throughout the VCS1703A genome, in eight different locations. The fimAB genes are located within a region of atypical trinucleotide composition, and microarray analysis indicated that fimA is divergent in all but two strains. fimA encodes the type IV fimbrial subunit; variation in fimA is the primary basis for D. nodosus serogroup typing (Claxton, 1983). Of the two strains which exhibit fimA conservation, one belongs to serogroup G, which is the same as the sequenced strain; the other belongs to serogroup I. fimB is divergent or missing in four of the strains, three of which are type II serogroup strains which characteristically lack fimB (Hobbs, 1991).

The genome contains 21 genes putatively involved in fimbrial biogenesis and 10 genes which appear to be involved in their regulation. Many of the absent genes are involved in regulation. Genes potentially involved in twitching motility (pilT, pilU) and its regulation (chpA, pilJ, pilI, pilG, pilH, ppk, fimX) were identified. In addition to putative tip adhesin (pilC) and secretin (pilQ) genes. pilQ was located in a region of atypical nucleotide composition, which was shown by microarray and PCR analysis to contain sequence variations.

PilQ appears to be the only outer membrane secretin encoded by D. nodosus. Since some type II secretion proteins share similarity to fimbrial proteins, we suggest that the type IV fimbriae may act as a secretion portal, expelling proteins through the motive force of fimbrial extension and retraction. We propose that in D. nodosus the type IV fimbrial secretin PilQ may act with the fimbrial biogenesis machinery as the only type II protein secretion system.

D. nodosus secretes three closely-related extracellular proteases (Riffkin et al, 1995) which are postulated to be involved in invasion and penetration by digesting the epidermal matrix of the hoof. Virulent isolates produce two acidic (AprV2, AprV5) proteases and one basic (BprV) protease, which are all members of the subtilisin protease family. These proteases are encoded within regions of unusual trinucleotide composition (AprV5, BprV and AprV2).

The major outer membrane protein of D. nodosus, Omp1, is encoded in a cluster of four homologous genes (Moses et al, 1995). This omp1 locus is located in a region of unusual trinucleotide composition, which is highly divergent in all strains examined by comparative hybridisation. This four-gene cluster is flanked on one side by two IS elements, belonging to the IS200 and IS605 families. These two elements were the only genes to give microarray intensity data indicative of being present in multiple copies.

The genome sequence also revealed potentially novel virulence factors, again associated with regions of atypical nucleotide composition. One of these regions encodes a putative secreted RTX-like toxin (DNO_{—}0334) and two ABC-family proteins which are probably involved in efflux of this protein (DNO_{—}0335-0336). RTX (repeats in toxin) toxins are large pore-forming protein toxins which damage host cells and tissues and also impair host defence mechanisms, and thus are important virulence factors in a variety of Gram-negative bacteria (Frey, 2002). A D. nodosus RTX-like toxin may potentially play a role in the necrotic sequelae of footrot.

Another region of atypical nucleotide composition encodes a putative large highly repetitive secreted protein (DNO_{—}0690; 32 nine amino acid repeat units), which may participate in adhesion to the extracellular matrix. This protein may be associated with virulence, since on the basis of the microarray data it is conserved in all virulent strains and absent in all benign strains.

Several other genes were identified which are homologous to virulence-associated genes in other organisms, or to genes in other organisms which encode protective antigens. These included two genes, DNO_{—}0466 and DNO 0650, which showed similarity to Shigella flexneri ispA and vacJ genes, which are involved in inter- and intracellular spreading of this pathogen (Mac Siomoin, 1996; Suzuki, 1994). DNO_{—}1067, a homologue of cell wall-associated hydrolases, was also identified. This gene product also exhibited similarity to GNA2001, a putative vaccine candidate from N. meningitidis (Pizza et al, 2000), and to the cell surface-associated Listeria monocytogenes protein P60, which promotes phagocyte invasion (Kuhn, 1989; Hess, 1995). Another gene, DNO_{—}0681, showed similarity to the D15 and Oma 87 proteins from Haemophilus influenzae and Pasteurella multocida, respectively, both of which are highly immunogenic and show some vaccine protection in animal models (Ruffolo, 1996: Loosmore, 1997) We identified a serine protease, DNO_{—}0902, which showed similarity to MucD from P. aeruginosa. MucD is involved in alginate biosynthesis and the production of extracellular toxins (Yorgey, 2001).

Proteins encoded by nucleic acid sequences which comprise regions of atypical nucleotide composition, which are otherwise associated with virulence, or which exhibit similarity to putative vaccine candidate proteins from other bacterial, species are considered likely to be useful as antigens for use in vaccines against D. nodosus. Thus for example PilT, PilU, ChpA, PilJ, PilI, PilG, PilH, Ppk, FimX, PilC, PilQ, RTX-like toxin (DNO_{—}0334), DNO_{—}0335-0336, putative large highly repetitive secreted protein (DNO_{—}0690), DNO_{—}0466, DNO_{—}0650, DNO_{—}1067, DNO_{—}0681 and DNO_{—}0902 are candidate antigens.

### Example 3

### Vaccine Candidate Selection and Analysis

We have taken a high-throughput reverse vaccinology approach to identify potential cross-protective vaccine candidates. A bioinformatic screen identified 99 predicted surface exposed or secreted proteins which were identified as potential vaccine candidates, 89 of which were able to be cloned into hexahistidine and NusA-tagged expression vectors. The recombinant proteins were screened against pooled immune and preimmune sera using Western blotting, and led to the identification of eight proteins which reacted to the immune antisera, but not to preimmune sera.

Putative vaccine candidates were identified from the D. nodosus genome on the basis of predicted cell localisation and the presence of signal and lipoprotein peptides and of the presence of fewer than two transmembrane domains, using the programs PSORTB (Gardy, 2003), SignalP (Neilsen, 1997), LipoP (Lu, 2004) and ProteomeAnalyst (Lu et al., 2004) to predict all outer membrane and secreted proteins and LipoP (Juncker et al., 2003) to predict all lipoproteins.

### Example 4

### Cloning of Candidate Antigen Genes

The large number of D. nodosus proteins which were identified as candidate antigens necessitated the adoption of a high-throughput cloning strategy. A recombinant cloning strategy was employed; this used the Gateway (Invitrogen Inc., Carlsbad, Calif.) cloning and expression system to clone PCR-amplified D. nodosus ORFs. Genes were cloned into the expression vectors pBAD-DEST49™, pDEST-17™ and a Gateway-adapted expression vector containing a NusA solubility tag, pLIC-Nus lacking signal peptides (Cabrita et al, 2006), using the Gateway™ recombination system (Invitrogen).

In general PCR primers were designed so as to amplify the gene region encoding the mature length protein, excluding the signal sequence, except where a signal sequence could not be predicted; in this case the primers were designed to encompass the entire gene. The primers used are listed in Table 2.

TABLE 2

Primers used for cloning of genes for candidate antigens

| | SEQ | | SEQ | |
|---|---|---|---|---|
| LOCUS | ID | | ID | |
| tag | NO: | 5′ primer | NO: | 3′ primer |
| DNO_0007 | 1 | CACCATGAGTCATAAAACAAAGCACAAATC | 2 | ATTCTCATCATCTGGCGATG |
| DNO_0012 | 3 | CACCATGGGCGTTGTGTTAAAAAATGAAG | 4 | TTTTTTAGCCGCTTCATCAG |
| DNO_0018 | 5 | CACCATGAGCGTAGATATCAACATTTTAGC | 6 | TAACGGCGTTGATAAACGCGAC |
| DNO_0024 | 7 | CACCATGGTGCAAAGCGACAGCATCA | 8 | ACGGGTAATACGAATACCACTTC |
| DNO_0025 | 9 | CACCATGGCGGATATGACGATTAAACC | 10 | AAACGTTTTAGAAAATTCCACAACCAC |
| DNO_0033 | 11 | CACCATGGCGGATAAATATCGCGTTGC | 12 | ATTAGCGGATTCCATTTCCAAC |
| DNO_0034 | 13 | CACCATGGAAACTTATCAGGTTGGCACC | 14 | ATTAGCGGATTCCATTTCCAAC |
| DNO_0043 | 15 | CACCATGCAAGAAATCGCCCAGCAGTA | 16 | TCGGCGGCAATCAGAAGCAAG |
| DNO_0067 | 17 | CACCATGAGCAGCACCAATCCCAGCCA | 18 | TTTAATCGCATTAATATCTTGGATA |
| DNO_0068 | 19 | CACCATGCTGCAAAATGTGGAAAAATCAC | 20 | GTTGAGTTCAAAACGTTTTCCTATGTTA |
| DNO_0081 | 21 | CACCATGGTTTCTAACGGTGGCGGTTTGA | 22 | ATAAACAGATTGATAAGCCGCC |
| DNO_0105 | 23 | CACCATGCAAGAAGAAAAAGCGCCGGC | 24 | TTCGGTAGCGATGCCCATTG |
| DNO_0110 | 25 | CACCATGATCCCTGCATACAACGACTAC | 26 | TGGAGTAGCTTCTTTCAATGCTTTAG |
| DNO_0125 | 27 | CACCATGGACACCATCATCACCCGTT | 28 | TGATGCCCGATGTTTATCGGGA |
| DNO_0128 | 29 | CACCATGCGCGCACCCGAACAAAACG | 30 | ATAGACACTCAAAATGTGATTTAATTC |
| DNO_0135 | 31 | CACCATGCATTTTCAATTAATTTATACGCC | 32 | CAATGATTCTGTTGCCAATTCCTCAGC |
| DNO_0155 | 33 | CACCATGTCTTCAACGAAAGACCCCTTTC | 34 | CGGACGGACAATCCAGTCTG |
| DNO_0194 | 35 | CACCATGCAAAGCGGCAATGTGATG | 36 | TAAAGCGGCAGGACCAGAAC |
| DNO_0235 | 37 | CACCATGATGTTGATTTGCAGTTTGAAGAAG | 38 | TACAATGTAAATGCTTGTTTGATCGAT |
| DNO_0245 | 39 | CACCATGGGTTTTCATCTGCGCGCGAC | 40 | TTGCGGTAATTGCTGTTGTTTC |
| DNO_0248 | 41 | CACCATGGATAACGCTAACAATCAATCCGC | 42 | TTTTTTAGCGTTTATTTCATC |
| DNO_0250 | 43 | CACCATGGAAACGTATAAAGTTGTTATTGAAC | 44 | AAAATAAGGTTTTTTCCCTACAATATTG |
| DNO_0255 | 45 | CACCATGAAAGAGCCGCTGCATTTAATG | 46 | CGGTTGCACGGCATACTCC |
| DNO_0296 | 47 | CACCATGATTGAATCGCCGGTGATGATTA | 48 | TTCATCAGTTGATGATTCTGTTTTTTCTGC |
| DNO_0300 | 49 | CACCATGATGGACGCCAGTCAGCAAC | 50 | ATAACCGTAACCGCCGCCGTA |
| DNO_0311 | 51 | CACCATGGCTGAAATTACGGATATCCG | 52 | TTGTTCTAAAAGCATATGTTGCGGTA |
| DNO_0316 | 53 | CACCATGATTGATAGCGCGCCTGCCGT | 54 | TTTTTCTGGTTGATAATAAGTCGGTTC |
| DNO_0318 | 55 | CACCATGGAAGAAGAAACTACGGCAGC | 56 | CTCGATTTTTAAAGAACGTAACGTTTC |
| DNO_0320 | 57 | CACCATGGATACTATGGAAAACCTAACTTC | 58 | GTTAATAACGCGTTGAACAGGGC |
| DNO_0333 | 59 | CACCATGCAAGATAGCGATGACGGCG | 60 | ATTCTGAAAAACGGCAATAACGC |
| DNO_0348 | 61 | CACCATGGGAACGATCACAGGGATTGATGC | 62 | GTTGTCGCGCAAATTAGAGA |
| DNO_0370 | 63 | CACCATGAGCGATGAAGCGGAAAAAATC | 64 | TTTTGAAACGTCAATAATACAACTGC |
| DNO_0377 | 65 | CACCATGAAAACTTATTTGGTCGGTACGGA | 66 | TCGCCAACGCGCAATAATTTGAC |
| DNO_0393 | 67 | CACCATGCTTCCTAGTTTGAATATTTACGCCA | 68 | CTGAAAACGCGCGCGAATTGCT |
| DNO_0406 | 69 | CACCATGCATTGGGATTTCAATGAAAAAAAC | 70 | TTTCTTTTTAGCGCTGGT |
| DNO_0424 | 71 | CACCATGGTCGCGCGCACGATTTGGTATTG | 72 | ATTAACGGATTGTTGTTCTCGATAAATC |
| DNO_0425 | 73 | CACCATGCAACCGCGAGCAATTGCTAC | 74 | ATTAATAAAATAAACTTCTTTCAACGC |
| DNO_0452 | 75 | CACCATGGAAACGCTGCGCCGCGCGA | 76 | ACGCTCAATCCATAAATATTTACTATGC |
| DNO_0462 | 77 | CACCATGGGCGAAAAAGATGCGCAGTATTA | 78 | TTTACCGAATTTTTTCAGCG |
| DNO_0484 | 79 | CACCATGCAATTGTTTCAAACGGCAGAGC | 80 | ATGTTCGCCGCCGACCACATC |
| DNO_0494 | 81 | CACCATGGAAAATACCACAACCCTTTATGGC | 82 | GAAATCGTGACGCAATCCTAC |
| DNO_0495 | 83 | CACCATGGAAAACACAACAACTCTTTATG | 84 | GAAATCGTGACGCAATCCTAAG |
| DNO_0496 | 85 | CACCATGGCAACACAAAAAACGGATTA | 86 | TGGCAAAAATGATTTTAAAGATTGC |
| DNO_0515 | 87 | CACCATGGTGTCGACGACGCAATCAACGC | 88 | TATTCCTAAAGATTTTGCTTGAGGTG |
| DNO_0519 | 89 | CACCATGTCAAGTTGGTTTTATGGTCATTC | 90 | TTGCGGTCGAAAAGCGACCA |
| DNO_0546 | 91 | CACCATGGCGGATCTGCCACAGATTCTGC | 92 | GGGTTTTTCCGGCAAAGTATAGC |
| DNO_0556 | 93 | CACCATGTCTTCAACGTATTACGACGC | 94 | TGATGCCGCTCGCTTCATTTTG |
| DNO_0571 | 95 | CACCATGACGGGCATGATGTATGGCAATC | 96 | GAATGACGCCGCGCGTTCGGCA |
| DNO_0603 | 97 | CACCATGGCCGTAAATTACGAATCCGC | 98 | GAATGCAACACTCCAACCTC |
| DNO_0605 | 99 | CACCATGGCTGAAAGTATCGTCAACTATGA | 100 | GAATGTCAAACTCCAACTATCG |
| DNO_0615 | 101 | CACCATGAGTCCTTCAAATACCCCGCA | 102 | AAACCAATGCATTTCTTGATGATTACGC |
| DNO_0632 | 103 | CACCATGCAATCTTCATTAGAAAAAAAACA | 104 | CTCCTGAAGACGGCAGCCCGTC |
| DNO_0644 | 105 | CACCATGCAAAAATTCACTGTTGTAAGCAC | 106 | CTTAGCGTTTTTATTGAGTGC |
| DNO_0650 | 107 | CACCATGAGCAGCGCCGTTGATGAACA | 108 | CTCGCTGGTTTCGGGCGCA |
| DNO_0681 | 109 | CACCATGCAATCGTTTTTGGTGGAAGATA | 110 | AAATGCCGTGCCCAACGTA |
| DNO_0700 | 111 | CACCATGCAAACTGCACCTGACGAGC | 112 | ATAGGTTTTATCTAAAAAGAAATCGTC |
| DNO_0712 | 113 | CACCATGGTTAATTTAGGTCCAATTCAGGTA | 114 | CCCCAATTTTTTCAATAAGTTTTGC |
| DNO_0725 | 115 | CACCATGGAATTGATGAGTTACCGCTGC | 116 | ACGTGCTTTACACATTTTATAAAGGATTTC |
| DNO_0861 | 117 | CACCATGCAAGAATCTTTAGAATCATTGCGCGA | 118 | TAAATCCAATTGCGTTCCAATG |
| DNO_0864 | 119 | CACCATGATCGGCTCCTTAGCTGATAA | 120 | ACGAGATACGGGAACCGGC |
| DNO_0873 | 121 | CACCATGATGCCGATTGCCATTGGCGGC | 122 | ATTGCTGATGGTGAATAAAGTAATC |
| DNO_0881 | 123 | CACCATGCATGATTTATGGGTTTGGGGTGA | 124 | TTTAACTTCAAATACTAAAGTTGCTTTATC |
| DNO_0890 | 125 | CACCATGATTCCAAATTATCAGCGTTAC | 126 | CCGTCCCCAACAACGAGTAGA |
| DNO_0895 | 127 | CACCATGGCGGACGCGAAAAAAGATAAAGC | 128 | CTTCAAAACAGATTCTTTGATTTCGC |
| DNO_0920 | 129 | CACCATGGATCCGCTGGCGAATTACACA | 130 | AAAACGCACTTCTTCGGGGAGC |
| DNO_0958 | 131 | CACCATGGGTTGCCATTCTTACCCCTC | 132 | ATACCTTGGTGAAGTTCCGATATA |
| DNO_1034 | 133 | CACCATGACCACAACTTATCCGCAAA | 134 | ATGAGAAGAGTGGCGCAACTG |
| DNO_1064 | 135 | CACCATGCAAAATCCGTTATTTTTATATCGTC | 136 | TTGAGTGGTCAGCCGCACTGC |
| DNO_1067 | 137 | CACCATGCAGCCGCAATTAACCTTATTA | 138 | GAGGCGCTTAATCGATGATTTTG |
| DNO_1115 | 139 | CACCATGACGACCGGTTCATTATTAGAAC | 140 | CAAACGGGTTTTTAAACGCGTC |
| DNO_1155 | 141 | CACCATGGATAACAAAAAAGAATCTCAAGA | 142 | CTTCATCGGAATTGGCGCAG |
| DNO_1167 | 143 | CACCATGGAAACTATGGTCAACTATGC | 144 | CAAAGTTAAACTCCAGCTCTTAATATAG |
| DNO_1170 | 145 | CACCATGATGCAAAATCAGCAATTATCTGA | 146 | TTGGATCGCTTTTGCGCC |
| DNO_1171 | 147 | CACCATGTCCAAGCCTTATAACATGGCGC | 148 | ATAAATAATCACCACACGCCGA |
| DNO_1190 | 149 | CACCATGAATCAATCGAAAGCAGATGC | 150 | GCGGGCGGTGAAATCTCGCG |
| DNO_1192 | 151 | CACCATGTCCTTTGTTCCTGATTACGA | 152 | TTTAATATCAGGTAATTGCGCGCCGTA |
| DNO_1241 | 153 | CACCATGGGTGGCAGCAGCGAGGAAT | 154 | AGGACGTTTGGCGCCGAATC |
| DNO_1302 | 155 | CACCATGGCACAAGAAGCTTATCAAGTGC | 156 | AAATGTTTTGCTCAATTCAATATAAGC |
| DNO_1310 | 157 | CACCATGTCGGGAATGCAGTTGGATCA | 158 | TAATTTTTTTATTGAGCTTCCGCGC |
| DNO_1317 | 159 | CACCATGGATGATGCCGTCATTGAA | 160 | TAAACGCGTTTCTAAAATTTTCCGCAAATC |
| DNO_1320 | 161 | CACCATGTTGCCGCTGAAAGAAGATTTTA | 162 | TAAAGCGGCAGGACCAGAAC |
| DNO_1322 | 163 | CACCATGCACATTATGGCGCCGGAAAG | 164 | TTTTTTAACGGTATGCGTTTTTTTCAGAG |
| DNO_1327 | 165 | CACCATGGTGACCGTGAATATTTATTTTCC | 166 | TTTTTGTTGCCACGAACCGTC |
| DNO_1329 | 167 | CACCATGTCAGAAGAAACTAAAAAAGAACCTC | 168 | TTGAACGGATTTTTCCGCTTCTTTAAC |
| DNO_1338 | 169 | CACCATGCTTCCCACGCCACCAGACATC | 170 | TTCTTTAATACCCAATTGATCCAGC |
| DNO_1343 | 171 | CACCATGGACGCGTATTGCGATGCATAC | 172 | GCTACCCCATTTGTTGATGATG |
| DNO_1345 | 173 | CACCATGGAAATCTATACCGTCGGCAC | 174 | TTCGAGCCATTTATCCACG |
| DNO_1348 | 175 | CACCATGAGCTACACTTTTCAAAATCTTCC | 176 | AGCGTTTGCCGCAGCAAATAATT |
| DNO_1351 | 177 | CACCATGGAAGAAGCTGCCGTGCCAG | 178 | TTTCAAGCCTTTTGCTTTGAG |

All 5′ PCR primers included a 5′-CACC tail to facilitate directional topoisomerase cloning. Where the primers were designed to amplify only the mature length portion without the signal sequence, the 5′-CACCATG tail was added so as to include a start codon. As the genes were to be expressed in-frame with either a C-terminal or an N-terminal tag, the native stop codon was not included in the reverse primers.

All PCR products were amplified from D. nodosus strain VCS1703A and cloned into the Gateway entry vector pENTR/SD/D-TOPO® (Invitrogen Inc., Carlsbad, Calif.). After the cloned genes were verified by sequencing and restriction digestion analysis, they were transferred by recombination using the LR Clonase kit (Invitrogen Inc., Carlsbad, Calif.) from the entry clone to the Invitrogen destination vectors pBAD-DEST49™, pDEST-17™ and a Gateway-adapted expression vector containing a NusA solubility tag, pLIC-Nus which lacks signal peptides (Cabrita et al., 2006). The expression host was E. coli BL21 codon plus (Stratagene). The gene loci encoding the expressed proteins are listed in Table 3.

TABLE 3
Loci of the D. nodosus proteins expressed by Gateway cloning

| Gene | | | pDEST | pDEST | pDEST | |
|---|---|---|---|---|---|---|
| locus | Stop | Start | 17 | 41BA | 49 | Mw |
| DNO_0007 | 9416 | 8034 | Yes | Yes | Yes | 54 |
| DNO_0012 | 13170 | 12181 | Yes | Yes | Yes | 37 |
| DNO_0018 | 20089 | 190061 | Yes | Yes | Yes | 39 |
| DNO_0024 | 29564 | 29205 | Yes | Yes | Yes | 14 |
| DNO_0025 | 31198 | 29747 | Yes | Yes | Yes | 58 |
| DNO_0033 | 39716 | 38976 | Yes | Yes | Yes | 29 |
| DNO_0034 | 40630 | 39902 | Yes | Yes | Yes | 29 |
| DNO_0043 | 48736 | 49614 | Yes | Yes | Yes | 34 |
| DNO_0067 | 65162 | 65749 | Yes | Yes | Yes | 22 |
| DNO_0068 | 65736 | 67439 | Yes | No | Yes | 69 |
| DNO_0081 | 80348 | 79671 | Yes | Yes | Yes | 26 |
| DNO_0105 | 109557 | 110990 | Yes | Yes | Yes | 54 |
| DNO_0110 | 117650 | 118144 | Yes | Yes | Yes | 17 |
| DNO_0125 | 130178 | 129189 | Yes | Yes | Yes | 38 |
| DNO_0128 | 131888 | 132634 | Yes | No | Yes | 30 |
| DNO_0135 | 140130 | 139333 | Yes | Yes | Yes | 30 |
| DNO_0155 | 160111 | 161163 | Yes | Yes | Yes | 46 |
| DNO_0194 | 208788 | 209279 | Yes | Yes | Yes | 18 |
| DNO_0235 | 255637 | 254189 | Yes | Yes | Yes | 54 |
| DNO_0245 | 266726 | 267220 | Yes | Yes | Yes | 19 |
| DNO_0248 | 271510 | 272541 | Yes | Yes | Yes | 37 |
| DNO_0250 | 273969 | 273496 | Yes | Yes | Yes | 19 |
| DNO_0255 | 281032 | 281961 | Yes | Yes | Yes | 34 |
| DNO_0296 | 312988 | 314325 | Yes | Yes | Yes | 52 |
| DNO_0300 | 318254 | 317880 | Yes | Yes | Yes | 14 |
| DNO_0311 | 331362 | 329908 | Yes | Yes | Yes | 54 |
| DNO_0316 | 337224 | 336643 | Yes | Yes | Yes | 21 |
| DNO_0318 | 339031 | 338072 | Yes | Yes | Yes | 37 |
| DNO_0320 | 340376 | 340984 | Yes | Yes | Yes | 22 |
| DNO_0333 | 358405 | 357509 | Yes | Yes | Yes | 33 |
| DNO_0348 | 379691 | 377487 | Yes | Yes | Yes | 83 |
| DNO_0370 | 410834 | 411358 | Yes | Yes | Yes | 20 |
| DNO_0377 | 417108 | 416362 | Yes | Yes | Yes | 28 |
| DNO_0393 | 438036 | 437011 | Yes | Yes | No | 36 |
| DNO_0406 | 450314 | 450565 | Yes | Yes | Yes | 29 |
| DNO_0424 | 37571 | 38413 | Yes | Yes | Yes | 35 |
| DNO_0425 | 37578 | 37153 | Yes | Yes | Yes | 18 |
| DNO_0452 | 493370 | 494944 | Yes | Yes | Yes | 62 |
| DNO_0462 | 501199 | 502290 | Yes | Yes | Yes | 43 |
| DNO_0484 | 520815 | 520123 | Yes | Yes | Yes | 28 |
| DNO_0494 | 530026 | 528896 | Yes | Yes | Yes | 43 |
| DNO_0495 | 531263 | 530286 | Yes | Yes | Yes | 36 |
| DNO_0496 | 531324 | 531890 | Yes | Yes | Yes | 22 |
| DNO_0515 | 551692 | 550931 | Yes | Yes | Yes | 30 |
| DNO_0519 | 555758 | 554613 | Yes | Yes | Yes | 41 |
| DNO_0546 | 584467 | 582782 | Yes | Yes | Yes | 63 |
| DNO_0556 | 594317 | 593673 | Yes | Yes | Yes | 27 |
| DNO_0571 | 609364 | 608477 | Yes | Yes | Yes | 34 |
| DNO_0603 | 641765 | 639978 | Yes | Yes | Yes | 67 |
| DNO_0605 | 644789 | 642978 | Yes | No | Yes | 64 |
| DNO_0615 | 658109 | 657873 | Yes | No | Yes | 10 |
| DNO_0632 | 677212 | 677610 | Yes | Yes | Yes | 14 |
| DNO_0644 | 688490 | 689392 | Yes | Yes | Yes | 34 |
| DNO_0650 | 694960 | 695712 | Yes | Yes | Yes | 29 |
| DNO_0681 | 730295 | 727995 | Yes | Yes | Yes | 89 |
| DNO_0700 | 755115 | 754651 | Yes | Yes | Yes | 19 |
| DNO_0712 | 765921 | 768800 | Yes | Yes | Yes | 105 |
| DNO_0725 | 778217 | 778909 | Yes | Yes | Yes | 29 |
| DNO_0861 | 901013 | 902812 | Yes | Yes | Yes | 69 |
| DNO_0864 | 905194 | 905433 | Yes | Yes | Yes | 9 |
| DNO_0873 | 915475 | 914867 | Yes | Yes | Yes | 22 |
| DNO_0881 | 923444 | 922683 | Yes | Yes | Yes | 30 |
| DNO_0890 | 931566 | 932021 | Yes | Yes | Yes | 18 |
| DNO_0895 | 934967 | 938785 | Yes | No | Yes | 143 |
| DNO_0920 | 963669 | 961477 | Yes | Yes | Yes | 88 |
| DNO_0958 | 997560 | 998261 | Yes | Yes | Yes | 27 |
| DNO_1034 | 1069598 | 1067601 | Yes | Yes | No | 76 |
| DNO_1064 | 1103515 | 1103183 | Yes | Yes | Yes | 13 |
| DNO_1067 | 1106171 | 1105557 | Yes | Yes | Yes | 23 |
| DNO_1115 | 1162485 | 1161418 | Yes | Yes | Yes | 41 |
| DNO_1155 | 1200411 | 1201931 | Yes | Yes | Yes | 59 |
| DNO_1167 | 1215097 | 1216902 | Yes | Yes | Yes | 66 |
| DNO_1170 | 1217442 | 1217999 | Yes | Yes | Yes | 21 |
| DNO_1171 | 1218024 | 1218608 | Yes | Yes | Yes | 22 |
| DNO_1190 | 1238072 | 1236759 | Yes | Yes | Yes | 49 |
| DNO_1192 | 1242646 | 1241243 | Yes | Yes | Yes | 53 |
| DNO_1241 | 1283149 | 1284618 | Yes | Yes | Yes | 57 |
| DNO_1302 | 1327812 | 1329248 | Yes | Yes | Yes | 57 |
| DNO_1310 | 1337830 | 1338666 | Yes | Yes | Yes | 34 |
| DNO_1317 | 1346829 | 1347515 | Yes | Yes | Yes | 28 |
| DNO_1320 | 1349062 | 1350333 | Yes | Yes | Yes | 49 |
| DNO_1322 | 1352918 | 1351854 | Yes | Yes | Yes | 42 |
| DNO_1327 | 1356754 | 1356161 | Yes | Yes | Yes | 23 |
| DNO_1329 | 1357841 | 1357317 | Yes | Yes | Yes | 19 |
| DNO_1338 | 1369083 | 1366930 | No | Yes | Yes | 87 |
| DNO_1343 | 1373146 | 1373895 | Yes | Yes | Yes | 29 |
| DNO_1345 | 1375607 | 1374870 | Yes | Yes | Yes | 29 |
| DNO_1348 | 1378439 | 1377828 | Yes | Yes | Yes | 24 |
| DNO_1351 | 1384148 | 1381989 | Yes | Yes | Yes | 85 |

### Example 5

### Assessment for Expression and Solubility

Each of the recombinant expression clones was assessed for levels of protein expression and solubility using an inclusion body assay developed on a liquid handling robot (TECAN).

The expressed proteins were purified as insoluble inclusion bodies. Strains were grown to mid-exponential phase (OD600=0.5) and induced for 4 h by the addition of arabinose to a final concentration of 0.2%. 1 ml induced cultures (Overnight Express™, Merck) were lysed with PopCulture and Lysonase™ (Merck) for 20 min at room temperature in a deep 24 well plate. Cell lysate (1 ml) was then added to a 96-well filter plate (AcroPrep™), and the solution was drawn through the filter under vacuum. The inclusion bodies were retained while soluble proteins passed through the filter. The retained inclusion bodies were washed once with Triton X-100 to remove any remaining soluble proteins, followed by two washes with phosphate buffer. The washed proteins were then denatured by the addition of 200 μl of 8M urea to each corresponding well, incubated for 2 hr at room temperature and collected under vacuum. Both soluble and insoluble fractions were then subjected to electrophoresis on an SDS-PAGE gel to assess the solubility of each protein.

### Example 6

### Identification of Antigens Recognised by Sheep Sera

To evaluate the range of D. nodosus proteins recognised by the sheep immune system serum, samples from sheep which had been repeatedly infected with the virulent D. nodosus strain VCS1001 serogroup A and then recovered after treatment with antibiotics were tested for reactivity against all the recombinant proteins by Western blot. So far 87 proteins have been purified and tested.

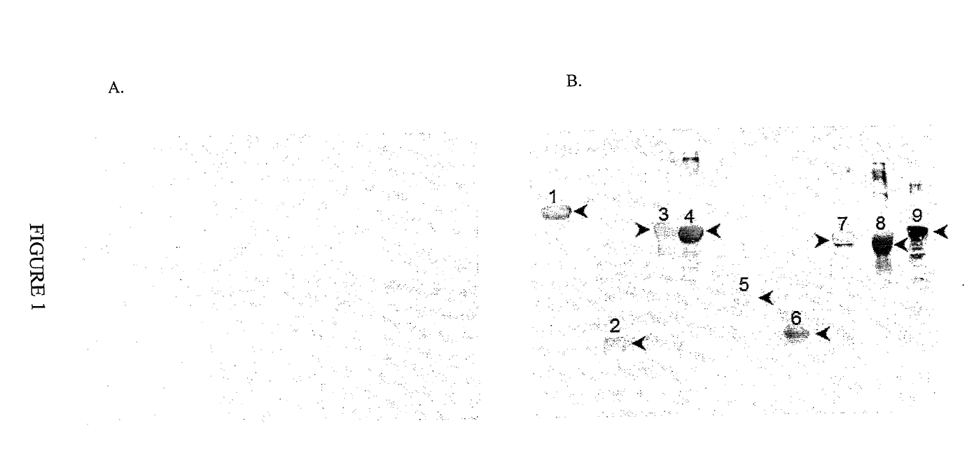

Recombinant proteins were screened against pooled sera from five infected sheep and five corresponding preimmune sera from the same sheep by Western blotting. Pooled serum samples from D. nodosus-infected sheep were reacted in immunoblot assays with comparable amounts of recombinant proteins. Five preimmune serum samples were reacted in parallel as controls. Fractions containing comparable levels of protein were separated by SDS-PAGE, transferred to nitrocellulose membranes and incubated with sera (1:200) before the addition of a peroxidase-conjugated anti-sheep antibody (diluted 1:800) (Chemicon). Positive reactions were detected using 4-chloro-1-naphthol. The results are summarized in Table 4, and illustrated in FIG. 1.

TABLE 4
Proteins showing specific reactivity to D. nodosus immune sera

| Gene/Antigen | |
|---|---|
| name | Putative Function of protein |
| DNO_0012 | peptidyl-prolyl cis-trans isomerase, FKBP-type |
| DNO_0033 | bacterial extracellular solute-binding family |
| | protein |
| DNO_0603 | acidic extracellular subtilisin-like protease |
| | precursor AprV5 |
| DNO_0605 | basic extracellular subtilisin-like protease |
| | precursor BprV |
| DNO_0644 | periplasmic iron-binding protein YfeA |
| DNO_0725 | potential adhesin complex protein |
| DNO_1167 | acidic extracellular subtilisin-like protease DNA |
| | and AprV2 |
| DNO_1241 | hypothetical lipoprotein |

The DNA and amino acid sequences of the proteins used in this example and their corresponding DNA sequences are as follows:

DNO_0012 Amino acid sequence
(SEQ ID NO: 179)
MKKTSLLLSAAIALSLTQVYAGVVLKNEGQKVGYAIGVDMGSSIAQLGISDGEELDFNAVILGL
RDAYQKKDLLLTQDEMTKTLQDFSEKRLQAMKKEMEKIAAEEAEKGKAFLEENAKKDGVITTES
GLQYKVVKKGTGAKPNSDDRVTVDYTGTLIDGTEFDSSKGREPITINVQDVIAGWVEGLQLMTE
GANYIFYIPSDLAYGSRGAGNAIPPNATLIFDVNLLKIEKNEAEAEADKKESIAKSINKSLEEA
TEIVKAEVEADKKESIAKSINKSLEEATETVKAEAEADKKEAIANSINKSLEEAAEAVKEVIEA
KPDEAAKK
DNO_0012 DNA sequence
(SEQ ID NO: 180)
ATGATGAAAAAAACTTCTTTACTATTATCAGCCGCGATTGCCTTGAGTTTGACGCAGGTT
TATGCCGGCGTTGTGTTAAAAAATGAAGGGCAAAAAGTGGGTTATGCCATCGGGGTGGAT
ATGGGAAGTTCCATTGCGCAATTAGGTATTTCTGATGGGGAAGAATTGGATTTTAATGCC
GTGATTTTGGGATTGCGCGATGCTTATCAAAAGAAAGATTTGCTTTTAACTCAAGATGAA
ATGACGAAAACGCTGCAAGATTTTTCTGAAAAACGTTTGCAAGCCATGAAAAAAGAAATG
GAAAAAATCGCCGCTGAAGAAGCAGAAAAAGGCAAAGCGTTTTTAGAAGAAAATGCGAAA
AAAGACGGCGTAATTACAACGGAATCTGGTTTGCAATATAAAGTTGTTAAAAAAGGAACC
GGCGCAAAACCCAATAGCGACGACCGCGTTACCGTTGATTACACCGGCACTTTGATTGAC
GGCACCGAATTTGACAGCTCAAAAGGACGCGAACCTATTACGATTAACGTACAAGATGTT
ATTGCTGGTTGGGTTGAAGGTTTGCAACTGATGACCGAAGGCGCTAATTATATTTTCTAT
ATTCCGTCTGATTTAGCTTATGGCTCTCGCGGCGCCGGTAACGCTATTCCGCCTAATGCT
ACTTTGATTTTTGATGTGAATTTACTCAAAATTGAAAAAAATGAAGCGGAAGCCGAAGCT
GATAAAAAAGAATCGATTGCAAAATCCATCAATAAATCTTTAGAAGAAGCCACGGAAATC
GTAAAAGCAGAAGTGGAAGCTGACAAAAAAGAATCGATTGCAAAATCCATTAATAAATCT
TTGGAAGAAGCCACAGAAACCGTAAAAGCGGAAGCCGAAGCTGATAAAAAAGAAGCCATT
GCCAATTCTATTAATAAATCTTTAGAAGAAGCGGCGGAAGCGGTAAAAGAAGTGATCGAA
GCGAAACCTGATGAAGCGGCTAAAAAATAG
DNO_0033 Amino acid sequence
(SEQ ID NO: 181)
MKKLLLAALLSCSLPCLAADKYRVASHPTFAPYEFLDGNGIITGYDVDLIQEIAKDQGFEVEIH
NDKWEELLDKLNDGKRDMIVSSLMDTEERRQLADLSKPYSEYEQFTVFYKRPDLSINSLSDLSG
LNAGAEKGTANVERLKQAGANVTELDSNFEGFKAIIRGSIDAYYCDEASMAYILRGYKDKKLPI
RTFRLPKEPGKTVIAVKKGNAELLEKINRGIDNLRANGKLDELRKKWLEMESAN
DNO_0033 DNA sequence
(SEQ ID NO: 182)
ATGAAAAAATTATTATTGGCAGCATTATTGTCATGTAGTTTGCCCTGTTTGGCAGCGGAT
AAATATCGCGTTGCTAGTCATCCGACTTTCGCACCGTATGAGTTTTTAGACGGAAACGGC
ATCATCACCGGATATGACGTTGATCTCATTCAAGAAATTGCCAAAGATCAAGGCTTTGAA
GTTGAAATTCATAATGATAAATGGGAAGAACTGCTTGATAAATTAAATGACGGCAAACGC
GATATGATCGTTTCTTCCTTGATGGATACTGAAGAACGGCGCCAATTGGCTGATTTGAGT
AAGCCTTATTCTGAATATGAACAATTTACCGTGTTTTATAAAAGACCCGATCTGTCAATC
AATTCTTTATCCGATTTGTCCGGTTTAAATGCTGGTGCGGAAAAAGGTACGGCAAACGTT
GAACGGCTGAAACAAGCTGGCGCAAATGTTACTGAATTGGATAGTAATTTTGAAGGATTT
AAAGCCATTATTCGGGGTTCTATTGATGCTTACTATTGCGATGAAGCATCGATGGCTTAT
ATTTTGAGAGGATATAAAGATAAAAAATTGCCGATTCGCACTTTTAGATTGCCGAAAGAA
CCGGGAAAAACCGTTATCGCAGTTAAAAAAGGCAACGCGGAATTGCTCGAAAAAATCAAC
CGTGGCATCGATAATTTGCGCGCAAATGGAAAACTCGATGAGCTTCGTAAAAAATGGTTG
GAAATGGAATCCGCTAATTAA
DNO_0603 Amino acid sequence
(SEQ ID NO: 183)
MKQSGINGVKTLTLVVCAALASQAYAAVNYESANYIGSQPEGSVRFIIKYKDKSQSQQMMTNRS
TTSVMNNNNITIAGFNAQFVRTMTIGAGIFAVPDLKTTKEAHLVMDTIASNPDVEYVEVDRWLR
PFAAPNDPFYNDQWHYYSEYGVKADKVWDRGITGKGVTVAVVDTGIVNHPDLNANVIPGSGYDF
IQEAEIAQDGDGRDSNPADAGDWHSNWACGKYPDPRYEKRNSSWHGSHVAGTIAAVTNNRIGVS
GVAYDAKIVPVRVLGRCGGYNSDINEGMYWAAGGHIDGVPDNKHPAQVINMSLGGPGVCGSTEQ
TLINRATQLGATIIVAAGNDNIDAYGVTPASCDNILTVGATTSNGTRAYFSNHGSVVDISAPGA
GITSTVDSGARYPSGPSYSLMDGTSMATPHVAGVAALVISAANSVNREMTPAQVRDVLVRTVSS
FNGTPDRRIGAGIVDADAAVNAVLDGNVVERPIDELKPQAEYRNPQIKLIRDYQMMFSEIKVNG
RPGNTKFAVVKADIRHTDPSQLKLRLVSPKGYEYAVHYDNIKNKSSELITFPRDEQMNGYWRLK
IVDTKRGVTGYTRGWSVAF
DNO_0605 Amino acid sequence
(SEQ ID NO: 184)
MNLSNISAVKVLTLVVSAAIAGQVCAAESIVNYESANAISKQPEGSVRFIVKYKDGTPSSQGLK
TRSTTKVMASGMQVAGFEAQFVRTTGLGAGIFAVPELKTTKEAHLVMDTIASNPDVEFVEVDRL
AYPKAAPNDPSYRQQWHYFGNYGVKANKVWDRGFTGQGVVVSVVDTGILDHVDLNGNMLPGYDF
ISSAPNARDGDQRDNNPADEGDWFDNWDCGGYPDPRREKKFSTWHGSHVAGTIAAVTNNGVGVA
GVAYGAKVIPVRVLGKCGGYDSDITDGMYWSAGGHIDGVPDNQNPAQVVNMSLGGGGGCSQNSQ
RMIDKTTNLGALIVIAAGNENQDASRTWPSSCNNVLSVGATTPKGKRAPFSNYGARVHLAAPGT
NILSTIDVGQAGPVRSSYGMKAGTSMAAPHVSGVAALVISAANSIGKTLTPSELSDILVRTTSR
FNGRLDRGLGSGIVDANAAVNAVLGDQNRAQPRPPVNQPINSGNKVYRSDRRVAIRDLRSVTSG
IRVNDQARVGSANITLTLDIRHGDRSQLAVELIAPSGRVYPIYHDGKRQPNIVGPATFSVKNER
LQGTWTLKVTDKARGVTGSIDSWSLTF
DNO_0644 Amino acid sequence
(SEQ ID NO: 185)
MKKSFLNSMMVLILTFFCSAAMAQKFTVVSTFTVIADIAQNIAGEHADVFSITKAGAEIHDYEP
TPHDLVTAQKAQLLLHNGLQLERWFERFHQSLKNVPAVTVSAGVVPIPIEGDSGIPNPHAWMSI
DNALIYVDNITQALQKYDPEHANDYAENARRYKEKIQALDTHLQQAFAKIPEQQRWLVTTEGAF
SYLARDYHLREAYVWPVNSEQEGTPQQIADLIATVRKHQIPVVFSESTLSDKHAKVVAKETGAQ
YGGVLYVDSLSTADGAVPTYLDLLKVTTDTIISGFESALNKNAK
DNO_0644 DNA sequence
(SEQ ID NO: 186)
ATGAAAAAATCATTTCTTAACTCAATGATGGTACTCATTTTGACGTTTTTTTGCTCGGCA
GCGATGGCGCAAAAATTCACTGTTGTAAGCACATTCACCGTCATTGCTGATATTGCTCAA
AATATTGCTGGTGAACACGCTGATGTTTTTTCTATTACTAAAGCTGGCGCTGAAATCCAC
GATTACGAACCTACGCCTCATGATTTGGTTACCGCCCAAAAAGCGCAACTTTTATTACAT
AATGGATTGCAACTAGAACGTTGGTTTGAGCGTTTTCATCAATCATTAAAAAATGTGCCA
GCAGTAACAGTCAGTGCGGGGGTAGTACCCATTCCTATTGAAGGTGATTCTGGCATTCCT
AATCCACATGCATGGATGTCGATCGATAATGCTTTGATTTATGTTGATAATATCACTCAA
GCACTGCAAAAATATGATCCTGAACACGCCAATGATTACGCTGAAAATGCACGGCGTTAC
AAAGAAAAAATTCAAGCGCTTGATACGCATCTTCAGCAAGCTTTTGCCAAAATTCCGGAA
CAACAACGTTGGTTAGTAACCACAGAAGGCGCTTTTTCCTATTTAGCACGCGATTATCAC
CTTCGTGAAGCCTATGTGTGGCCGGTTAACTCTGAGCAAGAAGGCACTCCACAACAAATT
GCCGATTTAATAGCAACTGTACGAAAACATCAAATTCCTGTCGTATTTAGCGAAAGTACC
CTGTCAGATAAACATGCAAAAGTTGTTGCTAAGGAAACTGGTGCTCAATATGGCGGCGTT
TTATACGTTGATTCACTTTCTACTGCTGATGGCGCGGTACCTACTTATTTAGATTTATTG
AAAGTAACTACCGATACAATAATCAGCGGTTTTGAATCTGCACTCAATAAAAACGCTAAG
TAA
DNO_0725 Amino acid sequence
(SEQ ID NO: 187)
MKKEQFLAAALLVFGFSTAAHAELMSYRCDQDRKIDVVYSFNSAGVPTKAEVMIQGKKRVLNYN
MDRSDNVDSFFNDKAGYELSSNIITRDDYHSSSIMIMSPKSEILFKNCSAAMSTIQPVMSEDAP
SVDANVVTSVKQANYVCQQGKKMKVEYGFNEVGIPVYALVNFKGTKLKLPYDLDQSSEASTFFT
KDGYVFAGDGMTVDTYRNSFMMLTAPSNEILYKMCKAR
DNO_0725 DNA sequence
(SEQ ID NO: 188)
ATGAAAAAATCATTTCTTAACTCAATGATGGTACTCATTTTGACGTTTTTTTGCTCGGCA
GCGATGGCGCAAAAATTCACTGTTGTAAGCACATTCACCGTCATTGCTGATATTGCTCAA
AATATTGCTGGTGAACACGCTGATGTTTTTTCTATTACTAAAGCTGGCGCTGAAATCCAC
GATTACGAACCTACGCCTCATGATTTGGTTACCGCCCAAAAAGCGCAACTTTTATTACAT
AATGGATTGCAACTAGAACGTTGGTTTGAGCGTTTTCATCAATCATTAAAAAATGTGCCA
GCAGTAACAGTCAGTGCGGGGGTAGTACCCATTCCTATTGAAGGTGATTCTGGCATTCCT
AATCCACATGCATGGATGTCGATCGATAATGCTTTGATTTATGTTGATAATATCACTCAA
GCACTGCAAAAATATGATCCTGAACACGCCAATGATTACGCTGAAAATGCACGGCGTTAC
AAAGAAAAAATTCAAGCGCTTGATACGCATCTTCAGCAAGCTTTTGCCAAAATTCCGGAA
CAACAACGTTGGTTAGTAACCACAGAAGGCGCTTTTTCCTATTTAGCACGCGATTATCAC
CTTCGTGAAGCCTATGTGTGGCCGGTTAACTCTGAGCAAGAAGGCACTCCACAACAAATT
GCCGATTTAATAGCAACTGTACGAAAACATCAAATTCCTGTCGTATTTAGCGAAAGTACC
CTGTCAGATAAACATGCAAAAGTTGTTGCTAAGGAAACTGGTGCTCAATATGGCGGCGTT
TTATACGTTGATTCACTTTCTACTGCTGATGGCGCGGTACCTACTTATTTAGATTTATTG
AAAGTAACTACCGATACAATAATCAGCGGTTTTGAATCTGCACTCAATAAAAACGCTAAG
TAA
DNO_1167 Amino acid sequence
(SEQ ID NO: 189)
MNKMALVVCAALVGQVASAETMVNYASAKAIGKQPAGSVRFIVKYKDNSQSSKDLKNRSTTKVM
ANGMQVAGFNAQFVRMTGAGAGIFSVPDLKTTKEAHLVMDTIASNPDVEFVEVDRIARPTAAPN
DQHYREQWHYFDRYGVKADKVWDMGFTGQNVVVAVVDTGILHHRDLNANVLPGYDFISNSQISL
DGDGRDADPFDEGDWFDNWACGGYPDPRKERSDSSWHGSHVAGTIAAVTNNRIGVAGVAYGAKV
VPVRALGRCGGYDSDISDGLYWAAGGRIAGIPENRNPAKVINMSLGSDGQCSYNAQTMIDRATR
LGALVVVAAGNENQNASNTWPTSCNNVLSVGATTSRGIRASFSNYGVDVDLAAPGQDILSTVDS
GTRRPVSDAYSFMAGTSMATPHVSGVAALVISAANSVNKNLTPAELKDVLVSTTSPFNGRLDRA
LGSGIVDAEAAVNSVLGNEGNNGRDDRRDNVAPVENARNYANNSIKFIRDYRLTSSVIEVEGRS
GAANGKINLALDIRHGNRSQLSIQLTSPAGHVYHINHDGARRPNLSGTVEIPVQNEQINGAWVL
QVGDHGRGATGYIKSWSLTL
DNO_1241 Amino acid sequence
(SEQ ID NO: 190)
MFTGNKFTVSVLSALTLAILTACGGSSEEFTLDRDHKWSHEPDRAKKEKRKHAELPAVNIPAPT
SFLEGAKKMKQTDDDNKEDKAVSKRQEGNVVTVVDHDYGYKTVKFNQSGMVDHLCMRVSESGVC
KIDDNKVANGIKWKEKEKKAAEGELAKIQKSIDDYMKTWDENHKNAEFGEREKELERIKELNKI
SDIEDRIEILEEELNELNEANKNMVFFLKSDGTQRLPNTSERILTKENYKNSYSKEKDIEDAGI
VPVAGFYDKNGDLRVQKGMVGREFDGIIVLENEVNQKKEVVKRSYLRDPVAAGWSYNTFGVFQD
STKSIERGYQSIGVKTRTSAQKRKEADESFEVDKGYENLPYYGRATYTGIGHAYHNDEQVTMNV
KVDADFHDRHLNFETSNATAHTFEKEGHLTRVRPDLDLKGAADWAADVADFKGKVHNVGGNLEG
QLEGSFYGPKAPEVGGVYGLHGKDKEGKPVNYVGGFGAKRP
DNO_1241 DNA sequence
(SEQ ID NO: 191)
ATGTTTACAGGAAATAAGTTTACAGTTTCAGTGTTATCAGCTTTAACGCTGGCAATTTTG
ACGGCATGCGGTGGCAGCAGCGAGGAATTCACCCTAGATAGAGATCATAAATGGTCACAT
GAACCAGATCGCGCAAAAAAAGAAAAAAGAAAACATGCGGAATTACCGGCGGTTAATATC
CCTGCGCCAACAAGTTTTTTAGAGGGCGCTAAAAAAATGAAACAAACTGATGACGATAAT
AAAGAAGATAAAGCAGTTTCAAAGAGACAAGAAGGCAACGTGGTGACTGTGGTTGATCAT
GATTATGGTTATAAAACAGTTAAGTTTAATCAATCAGGTATGGTTGACCATTTATGTATG
AGAGTAAGTGAATCAGGTGTATGCAAGATTGATGATAACAAAGTTGCTAACGGTATCAAA
AATAAGGAAAAAGAGAAAAAAGCTGCGGAAGGGGAACTCGCTAAAATTCAGAAAAGTATT
GATGACTACATGAAGACATGGGATGAGAATCATAAAAACGCTGAATTTGGTGAGCGTGAA
AAAGAGCTTGAGCGGATAAAGGAACTGAATAAGATCAGTGATATTGAAGATCGTATTGAA
ATATTAGAAGAAGAACTAAATGAGCTGAATGAAGCAAATAAAAATATGGTTTTCTTTTTA
AAATCAGATGGAACACAACGTCTTCCAAATACATCAGAGAGAATCTTGACAAAAGAGAAC
TATAAAAATAGTTACAGCAAAGAAAAAGATATTGAAGATGCTGGTATTGTTCCCGTTGCT
GGATTTTACGATAAAAATGGCGATTTAAGAGTGCAAAAGGGCATGGTTGGTAGAGAATTC
GATGGCATTATCGTGCTTGAAAATGAAGTGAATCAAAAAAAAGAAGTGGTTAAACGCTCT
TACTTACGCGATCCGGTAGCGGCAGGCTGGAGTTATAACACTTTTGGCGTTTTCCAAGAT
TCAACGAAGAGTATTGAACGCGGTTACCAAAGTATTGGTGTTAAAACCCGAACCAGTGCT
CAAAAAAGAAAAGAAGCTGACGAAAGCTTTGAAGTTGATAAAGGATATGAAAACTTACCC
TATTATGGACGCGCAACCTATACCGGTATCGGTCATGCTTATCACAATGATGAGCAAGTG
ACGATGAATGTAAAAGTTGATGCTGATTTCCATGATCGTCATTTAAATTTCGAAACTTCA
AATGCCACTGCGCATACTTTTGAAAAAGAAGGTCATTTGACACGTGTTCGTCCTGATTTA
GATTTAAAAGGCGCTGCGGATTGGGCAGCTGATGTTGCCGATTTTAAAGGTAAAGTGCAT
AACGTAGGCGGCAACTTAGAAGGTCAATTGGAAGGCAGTTTCTATGGACCAAAAGCGCCT
GAAGTGGGCGGCGTTTATGGTTTACACGGCAAAGATAAAGAAGGTAAACCAGTTAACTAT
GTTGGCGGATTCGGCGCCAAACGTCCTTGA

The immune sheep sera generated during D. nodosus strain VCS1703A infection recognised a total of 8 recombinant proteins, none of which were recognised by the corresponding preimmune sera. To our knowledge six of these antigens have not been previously identified as capable of eliciting immune responses from sheep. Thus we have demonstrated the utility of this approach for the identification of novel immunoreactive D. nodosus antigens.

Three of the eight proteins identified in this example were the acidic and basic extracellular subtilisin proteases of D. nodosus (DNO_{—}0603 (SEQ ID NO: 183), DNO_{—}0605 (SEQ ID NO: 184) and DNO_{—}1167 (SEQ ID NO: 189)), which have a predicted role in virulence via facilitating the digestion of the ovine hoof or connective tissue and thus aiding bacterial infiltration. Other proteins identified in this example include the Fur-regulated iron-binding protein YfeA (DNO_{—}0644; SEQ ID NO: 185), which has been shown by two-dimensional gel electrophoresis to be secreted (Parker, 2005), and a peptidyl-prolyl cis-trans isomerase (DNO_{—}0012; SEQ ID NO: 179) which is homologous to the macrophage infectivity potentiator (MIP) of Legionella pneumophila (40% identity). In L. pneumophila, MIP is an essential virulence factor which is antigenic, is located on the cell surface and is required for infection of protozoa and human macrophages (Cianciotto, 1992). A MIP homologue in Neisseria gonorrhoeae is also required for intracellular macrophage survival (Leuzzi, 2005).

Of the eight proteins identified, those encoded by genes DNO_{—}0603, DNO_{—}1167, DNO_{—}0605, and DNO_{—}0644 (SEQ ID NO: 186) are previously known, and those encoded by genes DNO_{—}0603, DNO_{—}1167 and DNO_{—}0605 have been proposed as vaccine antigens (U.S. Pat. No. 4,734,363 and WO88/09688). However, to our knowledge the remaining proteins identified in this example, namely DNO_{—}0012 (SEQ ID NO: 179), DNO_{—}0033 (SEQ ID NO: 181), DNO_{—}0725 (SEQ ID NO: 187), and DNO_{—}1241 (SEQ ID NO: 190), are novel per se, and YfeA (DNO_{—}0644; SEQ ID NO: 185) has not previously been shown or suggested to confer protective immunity, or proposed as a vaccine antigen.

The identification of these novel antigens constitutes a major advance towards the ultimate development of an efficacious cross-protective vaccine against ovine footrot. The novel antigens are being further tested in functional studies to elucidate the potential pathogenic role played by these immunogens in the establishment of disease. The results have important implications in understanding sheep immune responses to D. nodosus and in elucidating putative elements of a protective immune response.

### Example 7

### In Vitro Expression of D. nodosus Genes in the Presence of Hoof Powder

Little is known regarding D. nodosus gene expression in the ovine hoof. Moreover D. nodosus cell numbers in this site are probably not high enough to permit microarray analysis using in vivo material with current technologies. In an attempt to model in vivo conditions, and to examine subsequent changes in gene expression, transcriptional profiling was performed. RNA was extracted from cells grown on EYE agar, and compared by microarray analysis to RNA from cells grown on the same medium containing 2% ground ovine hoof powder.

RNA was extracted as described by Parker et al (2006) from two-day-old EYE agar cultures of D. nodosus, with and without 2% (w/v) ground sheep hoof (Thomas, 1958). RNA (3 μg) was labelled using the 3DNA Array 900 MPX labelling kit (Genisphere) and hybridisations were carried out as before (Parker et al, 2006), using the enhanced hybridisation buffer at 52° C. for cDNA hybridisation and the SDS-hybridisation buffer at 48° C. for 3DNA hybridisation.

Slides were scanned and analysed on four biological repeat samples with two dye swaps. Quantitative real-time RT-PCR (QRT-PCR) was performed on an ABI PRISM 7700 sequence detector using four biological replicates performed in triplicate, and the results are shown in Table 5. Statistical analysis was performed using a two-tailed student's t-test.

Microarray data have been deposited at the National Center for Biotechnology Information's Gene Expression Omnibus (GEO) database (http://www.ncbi.nlm.nih.gov/geo) under the series accession number GSE5166.

We identified 86 genes which were differentially expressed, and these are listed in Table 5. The genes identified in Table 6 represent a subset of those listed in Table 5. These genes covered a wide spectrum of functions, consistent with a growth-change experiment. Down-regulation of the global regulator integration host factor (IHF) was observed; conversely, increased expression was detected for the twitching motility gene pilU and a potential high affinity zinc uptake gene, znuA. These and several other genes were validated by QRT-PCR, and the results are shown in Table 6; the oligonucleotide primers used for QRT-PCR are listed in Table 7. These results provide valuable leads to genes which are potentially expressed upon contact of D. nodosus with the ovine hoof.

TABLE 5
Genes of D. nodosus differentially expressed when the organism
is grown on hoof agar, as identified by microarray analysis

| Gene Locus | Description | Value | p value |
|---|---|---|---|
| DNO_0011 | universal stress family protein | 2.58649 | 0.00195312 |
| DNO_0017 | GTP pyrophosphokinase | 2.0243 | 1.19E−07 |
| DNO_0019 | aminopeptidase, M1 family | 2.32971 | 1.19E−07 |
| DNO_0020 | NADP-dependent malic enzyme | 3.25714 | 1.19E−07 |
| DNO_0021 | beta-hexosaminidase | 2.34494 | 0.000488281 |
| DNO_0030 | transcription termination factor | 0.414821 | 1.19E−07 |
| DNO_0031 | conserved hypothetical protein | 0.438369 | 0.000488281 |
| DNO_0037 | hypothetical protein | 2.3675 | 1.91E−06 |
| DNO_0043 | hypothetical protein | 2.17017 | 2.26E−06 |
| DNO_0057 | hypothetical protein | 0.350972 | 0.000488281 |
| DNO_0063 | peptidyl-tRNA hydrolase | 0.440563 | 0.000488281 |
| DNO_0074 | enoyl-[acyl-carrier-protein] | 2.00899 | 2.38E−07 |
| | reductase | | |
| DNO_0077 | phosphoribosylaminoimidazole | 2.03848 | 9.54E−07 |
| | carboxylase, catalytic subunit | | |
| DNO_0106 | conserved hypothetical protein | 2.04969 | 3.58E−07 |
| DNO_0109 | fimbrial protein FimB | 0.380252 | 8.39E−05 |
| DNO_0112 | cytidylate kinase | 2.3152 | 6.68E−05 |
| DNO_0115 | Staphylococcal nuclease family | 0.358757 | 3.58E−07 |
| | protein | | |
| DNO_0116 | poly(A) polymerase PcnB | 0.454666 | 2.26E−06 |
| DNO_0118 | phosphoenolpyruvate-protein | 2.31783 | 4.77E−07 |
| | phosphotransferase | | |
| DNO_0135 | conserved hypothetical protein | 0.43516 | 1.19E−07 |
| DNO_0139 | NUDIX hydrolase domain | 0.465511 | 5.33E−05 |
| | protein | | |
| DNO_0155 | LysM domain protein | 2.28034 | 8.34E−07 |
| DNO_0166 | fructose-bisphosphate aldolase, | 2.26531 | 1.19E−07 |
| | class II | | |
| DNO_0181 | conserved hypothetical protein | 2.65669 | 1.19E−07 |
| | VrlK | | |
| DNO_0201 | peptidase T | 2.48641 | 0.000488281 |
| DNO_0219 | conserved hypothetical protein | 2.41926 | 0.000585556 |
| DNO_0220 | type I site-specific | 2.00918 | 0.000205159 |
| | deoxyribonuclease | | |
| DNO_0222 | type I restriction modification | 2.35787 | 3.81E−06 |
| | DNA specificity domain protein | | |
| DNO_0232 | hypothetical protein | 0.252776 | 0.000488281 |
| DNO_0268 | hypothetical protein | 0.273384 | 0.00488281 |
| DNO_0280 | virulence-associated protein | 0.448003 | 0.000488281 |
| | VapG1 | | |
| DNO_0282 | integrase A1 | 0.254733 | 0.000255585 |
| DNO_0333 | OmpA family protein | 0.406653 | 0.000488281 |
| DNO_0353 | RNA polymerase-binding | 0.388598 | 0.000488281 |
| | protein DksA | | |
| DNO_0370 | conserved hypothetical | 0.473265 | 0.000488281 |
| | lipoprotein | | |
| DNO_0391 | GTPase | 0.454645 | 8.85E−05 |
| DNO_0425 | conserved hypothetical protein | 0.499773 | 0.000488281 |
| DNO_0466 | intracellular septation protein A | 0.269956 | 1.43E−06 |
| DNO_0488 | GTP binding domain protein | 0.387934 | 0.000278115 |
| DNO_0523 | conserved hypothetical protein | 0.420691 | 0.000488281 |
| DNO_0552 | electron transport complex | 0.382056 | 1.19E−07 |
| | protein, D subunit | | |
| DNO_0564 | orotidine 5′-phosphate | 0.391516 | 1.19E−07 |
| | decarboxylase | | |
| DNO_0567 | conserved hypothetical protein | 0.447457 | 0.000488281 |
| DNO_0604 | lysyl-RNA synthetase | 0.378311 | 2.38E−07 |
| DNO_0606 | bacterial extracellular solute- | 0.466317 | 1.63E−05 |
| | binding family protein | | |
| DNO_0638 | hypothetical membrane protein | 0.332705 | 1.19E−07 |
| DNO_0639 | conserved hypothetical protein | 0.337417 | 4.77E−07 |
| DNO_0676 | twitching motility protein PilU | 2.07219 | 8.34E−07 |
| DNO_0707 | zinc uptake regulation protein | 0.443246 | 9.08E−05 |
| DNO_0736 | transcription termination factor | 0.392143 | 1.19E−07 |
| | Rho | | |
| DNO_0737 | thioredoxin I | 0.362944 | 0.000488281 |
| DNO_0764 | hypothetical protein | 2.56433 | 0.000107646 |
| DNO_0765 | hypothetical protein | 2.46077 | 1.19E−07 |
| DNO_0775 | conserved hypothetical protein | 2.01629 | 6.56E−06 |
| DNO_0776 | DNA replication protein DnaC | 3.03769 | 1.53E−05 |
| DNO_0779 | hypothetical protein | 0.496597 | 0.000488281 |
| DNO_0790 | hypothetical protein | 0.370534 | 0.000488281 |
| DNO_0803 | inositol monophosphatase | 0.439729 | 1.19E−06 |
| DNO_0879 | conserved hypothetical protein | 0.33152 | 9.54E−07 |
| DNO_0880 | formate/nitrite transporter | 0.483022 | 2.26E−06 |
| | family protein | | |
| DNO_0889 | high-affinity zinc uptake system | 5.059 | 3.81E−06 |
| | protein ZnuA | | |
| DNO_0902 | serine protease | 2.20401 | 0.000349283 |
| DNO_0928 | conserved hypothetical protein | 2.18447 | 1.67E−06 |
| DNO_0944 | hypothetical protein | 0.197109 | 1.53E−05 |
| DNO_0945 | polyprenyl synthase | 0.223567 | 1.19E−07 |
| DNO_0948 | tRNA modification GTPase | 0.414215 | 1.19E−07 |
| | TrmE | | |
| DNO_0949 | preprotein translocase subunit | 0.329517 | 2.98E−06 |
| | YidC | | |
| DNO_0950 | conserved hypothetical protein | 0.349673 | 0.000488281 |
| DNO_0952 | 50S ribosomal protein L34 | 0.365603 | 0.000488281 |
| DNO_0955 | DNA polymerase III, subunits | 0.463417 | 1.19E−07 |
| | gamma and tau | | |
| DNO_1017 | ATP-dependent RNA helicase | 0.346607 | 1.19E−07 |
| DNO_1023 | pyridoxamine 5′-phosphate | 2.50439 | 5.96E−07 |
| | oxidase | | |
| DNO_1045 | integrase A2 | 0.331732 | 1.19E−07 |
| DNO_1057 | integration host factor, alpha | 0.323678 | 0.000488281 |
| | subunit | | |
| DNO_1079 | DNA primase | 0.465712 | 0.000152588 |
| DNO_1082 | conserved hypothetical protein | 0.220464 | 0.000488281 |
| DNO_1178 | crossover junction | 2.21331 | 0.000488281 |
| | endodeoxyribonuclease RuvC | | |
| DNO_1179 | conserved hypothetical protein | 2.51217 | 1.19E−07 |
| DNO_1240 | YhbY family protein | 2.18399 | 1.19E−07 |
| DNO_1241 | hypothetical lipoprotein | 2.41645 | 4.77E−07 |
| DNO_1288 | preprotein translocase, SecE | 0.446131 | 1.91E−06 |
| | subunit | | |
| DNO_1311 | ribosomal large subunit | 2.15177 | 0.00012207 |
| | pseudouridine synthase, RluD | | |
| DNO_1321 | tyrosyl-tRNA synthetase | 2.21911 | 3.58E−07 |
| DNO_1346 | heat shock protein HslVU, | 2.07807 | 2.38E−07 |
| | protease HslV | | |
| DNO_1347 | heat shock protein HslVU, | 2.25833 | 1.19E−07 |
| | ATPase subunit HslU | | |
| DNO_1350 | aminotripeptidase | 2.27501 | 2.26E−06 |

TABLE 6
Genes of D. nodosus differentially expressed when the
organism is grown on hoof agar, validated with QRT-PCR

| | Microarray | | QRT-PCR | | |
|---|---|---|---|---|---|
| | Hoof/control | | Hoof/control | | |
| | ratio | p-value | ratio | SD | p-value |
| feoA | 0.54 | 1.81E−05 | 0.40 | 0.15 | 1.46E−02 |
| pilU | 2.07 | 8.43E−07 | 5.84 | 2.41 | 4.73E−04 |
| znuA | 5.06 | 3.81E−06 | 16.4 | 4.76 | 2.16E−05 |
| ihfA | 0.32 | 4.88E−04 | 0.21 | 0.12 | 7.88E−05 |
| hslU | 2.26 | 1.19E−07 | 1.72 | 0.72 | 6.92E−03 |

TABLE 7
Oligonucleotide primers used for QRT-PCR

| | Primer | SEQ ID | |
|---|---|---|---|
| Gene | number | NO: | Sequence 5′-3′ |
| feoA | JRP2876 | 192 | CGGCGTCGTTTGATGGAT |
| | JRP2877 | 193 | ACGGTGCAGGGCGAATAA |
| pilU | JRP2878 | 194 | CATGGTTGAAGTGCCAAATGTT |
| | JRP2879 | 195 | CGATCCCCGGCATATTTTTT |
| znuA | JRP2882 | 196 | CGTGGTGCGCGTCGAT |
| | JRP2883 | 197 | TGTTTGCGTAGCGCTTCAAT |
| ihfA | JRP2884 | 198 | CCGGCTTTGGTAATTTCGAA |
| | JRP2885 | 199 | TCTCCGGTTTTGGGATTGC |
| hslU | JRP2897 | 200 | CGCGAGCGCATTTTGC |
| | JRP2898 | 201 | TGGCGCTCATCGATTGATT |

### Example 7

### Bulk Expression and Purification of Antigens

The coding sequences of the genes listed in Tables 3, 4 and 5 are cloned into the expression vectors as listed in Table 3 or into other expression vectors which can lead to protein overexpression in E coli or other bacteria such as Ps. aeruginosa. To produce recombinant proteins, 200 ml cultures (Overnight Express™, Merck) containing ampicillin (100 μg/ml) are grown overnight at 28° C., with constant shaking at 250 rpm. The cells are collected by centrifugation at 3500 g for 10 minutes and resuspended in nickel affinity buffer (100 mM sodium phosphate buffer, pH 7.4 containing 150 mM NaCl and 10 mM imidazole). The cells are then lysed by sonication on ice for 6 rounds of 30 sec with a 10 mm sonication probe, interspersed with 30 sec rest intervals. After sonication the soluble and insoluble fractions are separated by centrifugation at 7500 g for 20 min.

For the soluble proteins, the soluble fraction prepared above is filtered through a 0.22 μm filter and loaded on to a HisTrap FF nickel affinity column (GE Healthcare—Life Sciences) at 1 ml/min. After washing the column with the nickel affinity buffer to remove non-specific proteins, the recombinant proteins is eluted from the Nickel affinity column with 100 mM sodium phosphate buffer, pH 7.4, containing 150 mM NaCl and 0.5 M imidazole. The eluted proteins are then loaded on to a HiLoad 16/60 Superdex 200 pg size exclusion chromatography column (GE Healthcare—Life Sciences) and the fractions containing the protein of interest are collected in 100 mM sodium phosphate buffer, pH 7.4, containing 150 mM NaCl.

For the insoluble proteins, the insoluble fraction is washed twice with 100 mM sodium phosphate buffer, pH 7.4, with 150 mM NaCl and 1 mM 2-mercaptoethanol, 1% (v/v) Triton X-100 to remove non-specific proteins. This fraction is pelleted by centrifugation at 7500 g for 20 min and washed in 100 mM sodium phosphate buffer, pH 7.4, with 150 mM NaCl with 1 mM 2-mercaptoethanol and the insoluble fraction again pelleted by centrifugation at 7500 g for 20 min. The pellet is resuspended in 100 mM sodium phosphate buffer, pH 7.4, with 150 mM NaCl, 10 mM 2-mercaptoethanol and 8M urea and mixed for one hour at room temperature. The proteins which are still insoluble after this incubation period are removed by centrifugation at 27000 g for 20 min, and the urea solubilized material is loaded on to a HisTrap FF column at 1 ml/min. After washing this column with 100 mM sodium phosphate buffer, pH 7.4, with 0.15 M NaCl, 1 mM 2-mercaptoethanol and 8M urea, the recombinant proteins are eluted with the same buffer containing 0.5 M imidazole. The eluted proteins are loaded on to a Hiload 16/60 desalting column (GE Healthcare-Life sciences) and the various fractions collected in 100 mM sodium phosphate buffer, pH 7.4, with 0.15 M NaCl, 1 mM 2-mercaptoethanol, and 8 M urea.

The various fractions from both methods are analysed by visualization of the recombinant proteins using SDS-PAGE gels, which are stained with Coomassie blue.

A total of 200 μg of each of the recombinant antigens is mixed together with an adjuvant such as Alhydrogel, and optionally emulsified in Freund's incomplete adjuvant prior to vaccination. Other suitable adjuvants are known, and the person skilled in the art will readily be able to test which formulations are most suitable.

Table 8 summarizes the D. nodosus genes which have been cloned, expressed and purified.

TABLE 8
D. nodosus proteins which have been cloned and expressed

| gid | chr | stop | start | who | locus_tag | ftype | gene | comment |
|---|---|---|---|---|---|---|---|---|
| D_nodosus | 1 | 9416 | 8034 | ZZ | DNO_0007 | CDS | | DN0065 VC002 pDEST-17 pLIC-Nus pBAD-DEST49 |
| D_nodosus | 1 | 13170 | 12181 | ZZ | DNO_0012 | CDS | | potential virulence DN0068 VC003 pDEST-17 pLic- |
| | | | | | | | | Nus pBAD-DEST49 |
| D_nodosus | 1 | 20089 | 19061 | ZZ | DNO_0018 | CDS | | DN0072 VC004 pDEST-17 pLic-Nus pBAD-DEST49 |
| D_nodosus | 1 | 29564 | 29205 | ZZ | DNO_0024 | CDS | | DN0079 VC65 pDEST-17 pLic-Nus pBAD-DEST49 |
| D_nodosus | 1 | 31198 | 29747 | ZZ | DNO_0025 | CDS | | DN0082 VC66 pDEST-17 pLic-Nus pBAD-DEST49 |
| D_nodosus | 1 | 39716 | 38976 | ZZ | DNO_0033 | CDS | | DN0087 VC67 pDEST-17 pLic-Nus pBAD-DEST49 |
| D_nodosus | 1 | 40630 | 39902 | ZZ | DNO_0034 | CDS | | DN0088 VC68 pDEST-17 pLic-Nus pBAD-DEST49 |
| D_nodosus | 1 | 48736 | 49614 | ZZ | DNO_0043 | CDS | | DN0098 VC70 pDEST-17 pLic-Nus pBAD-DEST49 |
| D_nodosus | 1 | 65162 | 65749 | ZZ | DNO_0067 | CDS | | DN0113 VC31 pDEST-17 pLic-Nus pBAD-DEST49 |
| D_nodosus | 1 | 65736 | 67439 | ZZ | DNO_0068 | CDS | | DN0114 VC72 pDEST-17 pBAD-DEST49 |
| D_nodosus | 1 | 80348 | 79671 | ZZ | DNO_0081 | CDS | | DN0126 VC005 pDEST-17 pLic-Nus pBAD-DEST49 |
| D_nodosus | 1 | 109557 | 110990 | ZZ | DNO_0105 | CDS | | DN0150 VC73 pDEST-17 pLic-Nus pBAD-DEST49 |
| D_nodosus | 1 | 117650 | 118144 | ZZ | DNO_0110 | CDS | fimA | fimbriae ~~~ potential virulence DN0156 VC006 |
| | | | | | | | | pDEST-17 pLic-Nus pBAD-DEST49 |
| D_nodosus | 1 | 130178 | 129189 | ZZ | DNO_0125 | CDS | | DN0172 VC74 pDEST-17 pLic-Nus pBAD-DEST49 |
| D_nodosus | 1 | 131888 | 132634 | ZZ | DNO_0128 | CDS | | DN0178 VC75 pDEST-17 pBAD-DEST49 |
| D_nodosus | 1 | 140130 | 139333 | ZZ | DNO_0135 | CDS | | interest Fur regulated DN0182 VC007 pDEST-17 |
| | | | | | | | | pLic-Nus pBAD-DEST49 |
| D_nodosus | 1 | 160111 | 161163 | ZZ | DNO_0155 | CDS | | DN0203 VC76 pDEST-17 pLic-Nus pBAD-DEST49 |
| D_nodosus | 1 | 208788 | 209279 | ZZ | DNO_0194 | CDS | | DN0237 VC33 pDEST-17 pLic-Nus pBAD-DEST49 |
| D_nodosus | 1 | 255637 | 254189 | ZZ | DNO_0235 | CDS | | potential virulence DN0275 VC008 pDEST-17 pLic- |
| | | | | | | | | Nus pBAD-DEST49 |
| D_nodosus | 1 | 266726 | 267220 | ZZ | DNO_0245 | CDS | | DN0286 VC78 pDEST-17 pLic-Nus pBAD-DEST49 |
| D_nodosus | 1 | 271510 | 272541 | ZZ | DNO_0248 | CDS | | potential virulence DN0289 VC35 pDEST-17 pLic- |
| | | | | | | | | Nus pBAD-DEST49 |
| D_nodosus | 1 | 273969 | 273496 | ZZ | DNO_0250 | CDS | | DN0291 VC79 pDEST-17 pLic-Nus pBAD-DEST49 |
| D_nodosus | 1 | 281032 | 281961 | ZZ | DNO_0255 | CDS | | DN0297 VC80 pDEST-17 pLic-Nus pBAD-DEST49 |
| D_nodosus | 1 | 312988 | 314325 | ZZ | DNO_0296 | CDS | | DN0333 VC81 pDEST-17 pLic-Nus pBAD-DEST49 |
| D_nodosus | 1 | 318254 | 317880 | ZZ | DNO_0300 | CDS | | DN0337 VC82 pDEST-17 pLic-Nus pBAD-DEST49 |
| D_nodosus | 1 | 331362 | 329908 | ZZ | DNO_0311 | CDS | | DN0348 VC83 pDEST-17 pLic-Nus pBAD-DEST49 |
| D_nodosus | 1 | 337224 | 336643 | ZZ | DNO_0316 | CDS | | DN0353 VC84 pDEST-17 pLic-Nus pBAD-DEST49 |
| D_nodosus | 1 | 339031 | 338072 | ZZ | DNO_0318 | CDS | | DN0355 VC85 pDEST-17 pLic-Nus pBAD-DEST49 |
| D_nodosus | 1 | 340376 | 340984 | ZZ | DNO_0320 | CDS | | DN0357 VC9 pDEST-17 pLic-Nus pBAD-DEST49 |
| D_nodosus | 1 | 358405 | 357509 | ZZ | DNO_0333 | CDS | | DN0370 VC10 pDEST-17 pLic-Nus pBAD-DEST49 |
| D_nodosus | 1 | 379691 | 377487 | ZZ | DNO_0348 | CDS | pilQ | fimbriae DN0385 VC11 pDEST-17 pLic-Nus pBAD- |
| | | | | | | | | DEST49 |
| D_nodosus | 1 | 410834 | 411358 | ZZ | DNO_0370 | CDS | | DN0408 VC38 pDEST-17 pLic-Nus pBAD-DEST49 |
| D_nodosus | 1 | 417108 | 416362 | ZZ | DNO_0377 | CDS | | DN0415 VC89 pDEST-17 pLic-Nus pBAD-DEST49 |
| D_nodosus | 1 | 438036 | 437011 | ZZ | DNO_0393 | CDS | | DN0428 VC90 pDEST-17 pLic-Nus |
| D_nodosus | 1 | 450314 | 450565 | ZZ | DNO_0406 | CDS | | DN0442 VC92 |
| D_nodosus | 1 | 464201 | 465043 | ZZ | DNO_0424 | CDS | | DN0458 VC94 pDEST-17 pLic-Nus pBAD-DEST49 |
| D_nodosus | 1 | 464208 | 463783 | ZZ | DNO_0425 | CDS | | DN0457 VC95 pDEST-17 pLic-Nus pBAD-DEST49 |
| D_nodosus | 1 | 493370 | 494944 | ZZ | DNO_0452 | CDS | | DN0481 VC96 pDEST-17 pLic-Nus pBAD-DEST49 |
| D_nodosus | 1 | 501199 | 502290 | ZZ | DNO_0462 | CDS | | DN0490 VC97 pDEST-17 pLic-Nus pBAD-DEST49 |
| D_nodosus | 1 | 520815 | 520123 | ZZ | DNO_0484 | CDS | | DN0502 VC98 pDEST-17 pLic-Nus pBAD-DEST49 |
| D_nodosus | 1 | 530026 | 528896 | ZZ | DNO_0494 | CDS | | DN0522 VC16 pDEST-17 pLic-Nus pBAD-DEST49 |
| D_nodosus | 1 | 531263 | 530286 | ZZ | DNO_0495 | CDS | | DN0523 VC17 pDEST-17 pLic-Nus pBAD-DEST49 |
| D_nodosus | 1 | 531324 | 531890 | ZZ | DNO_0496 | CDS | | DN0524 VC99 pDEST-17 pLic-Nus pBAD-DEST49 |
| D_nodosus | 1 | 551692 | 550931 | ZZ | DNO_0515 | CDS | | fimbriae DN0544 VC103 pDEST-17 pLic-Nus pBAD- |
| | | | | | | | | DEST49 |
| D_nodosus | 1 | 555758 | 554613 | ZZ | DNO_0519 | CDS | | DN0548 VC39 pDEST-17 pLic-Nus pBAD-DEST49 |
| D_nodosus | 1 | 584467 | 582782 | ZZ | DNO_0546 | CDS | | DN0576 VC40 pDEST-17 pLic-Nus pBAD-DEST49 |
| D_nodosus | 1 | 594317 | 593673 | ZZ | DNO_0556 | CDS | | DN0586 VC105 pDEST-17 pLic-Nus pBAD-DEST49 |
| D_nodosus | 1 | 609364 | 608477 | ZZ | DNO_0571 | CDS | | DN0601 VC41 pDEST-17 pLic-Nus pBAD-DEST49 |
| D_nodosus | 1 | 641765 | 639978 | ZZ | DNO_0603 | CDS | aprV5 | potential virulence DN0632 VC18 pDEST-17 pLic- |
| | | | | | | | | Nus pBAD-DEST49 |
| D_nodosus | 1 | 644789 | 642978 | ZZ | DNO_0605 | CDS | bprV | potential virulence DN0634 VC19 pDEST-17 pBAD- |
| | | | | | | | | DEST49 |
| D_nodosus | 1 | 658109 | 657873 | ZZ | DNO_0615 | CDS | | DN0643 VC112 pDEST-17 pBAD-DEST49 |
| D_nodosus | 1 | 677212 | 677610 | ZZ | DNO_0632 | CDS | | DN0659 VC114 pDEST-17 pLic-Nus pBAD-DEST49 |
| D_nodosus | 1 | 688490 | 689392 | ZZ | DNO_0644 | CDS | yfeA | DN0671 VC20 pDEST-17 pLic-Nus pBAD-DEST49 |
| D_nodosus | 1 | 694960 | 695712 | ZZ | DNO_0650 | CDS | | potential virulence DN0677 VC42 pDEST-17 pLIC- |
| | | | | | | | | Nus pBAD-DEST49 |
| D_nodosus | 1 | 730295 | 727995 | ZZ | DNO_0681 | CDS | | potential virulence DN0708 VC21 pDEST-17 pLic- |
| | | | | | | | | Nus pBAD-DEST49 |
| D_nodosus | 1 | 755115 | 754651 | ZZ | DNO_0700 | CDS | | DN0727 VC45 pDEST-17 pLic-Nus pBAD-DEST49 |
| D_nodosus | 1 | 765921 | 768800 | ZZ | DNO_0712 | CDS | fimV | fimbriae DN0740 VC119 pDEST-17 pLic-Nus pBAD- |
| | | | | | | | | DEST49 |
| D_nodosus | 1 | 778217 | 778909 | ZZ | DNO_0725 | CDS | | potential virulence DN0753 VC46 pDEST-17 pLic- |
| | | | | | | | | Nus pBAD-DEST49 |
| D_nodosus | 1 | 901013 | 902812 | ZZ | DNO_0861 | CDS | | potential virulence DN0883 VC22 pDEST-17 pLic- |
| | | | | | | | | Nus pBAD-DEST49 |
| D_nodosus | 1 | 905194 | 905433 | ZZ | DNO_0864 | CDS | | DN0886 VC128 pDEST-17 pLic-Nus pBAD-DEST49 |
| D_nodosus | 1 | 915475 | 914867 | ZZ | DNO_0873 | CDS | | DN0895 VC47 pDEST-17 pLic-Nus pBAD-DEST49 |
| D_nodosus | 1 | 923444 | 922683 | ZZ | DNO_0881 | CDS | | interest Fur regulated DN0903 VC23 pDEST-17 pLic- |
| | | | | | | | | Nus pBAD-DEST49 |
| D_nodosus | 1 | 931566 | 932021 | ZZ | DNO_0890 | CDS | | fimbriae DN0908 pDEST-17 pLic-Nus pBAD-DEST49 |
| D_nodosus | 1 | 934967 | 938785 | ZZ | DNO_0895 | CDS | pilC | fimbriae DN0913 VC49 pDEST-17 pBAD-DEST49 |
| D_nodosus | 1 | 963669 | 961477 | ZZ | DNO_0920 | CDS | ostA | DN0937 VC136 pDEST-17 pLic-Nus pBAD-DEST49 |
| D_nodosus | 1 | 997560 | 998261 | ZZ | DNO_0958 | CDS | | DN0969 VC51 pDEST-17 pLic-Nus pBAD-DEST49 |
| D_nodosus | 1 | 1069598 | 1067601 | ZZ | DNO_1034 | CDS | | DN1045 VC155 pDEST-17 pLic-Nus |
| D_nodosus | 1 | 1103515 | 1103183 | ZZ | DNO_1064 | CDS | | DN1074 VC52 pDEST-17 pLic-Nus pBAD-DEST49 |
| D_nodosus | 1 | 1106171 | 1105557 | ZZ | DNO_1067 | CDS | | potential virulence DN1077 VC53 pDEST-17 pLic- |
| | | | | | | | | Nus pBAD-DEST49 |
| D_nodosus | 1 | 1162485 | 1161418 | ZZ | DNO_1115 | CDS | | DN1123 VC55 pDEST-17 pLic-Nus pBAD-DEST49 |
| D_nodosus | 1 | 1200411 | 1201931 | ZZ | DNO_1155 | CDS | | DN1162 VC164 pDEST-17 pLic-Nus pBAD-DEST49 |
| D_nodosus | 1 | 1215097 | 1216902 | ZZ | DNO_1167 | CDS | aprV2 | potential virulence DN1174 VC24 pDEST-17 pLic- |
| | | | | | | | | Nus pBAD-DEST49 |
| D_nodosus | 1 | 1217442 | 1217999 | ZZ | DNO_1170 | CDS | | DN1175 VC165 pDEST-17 pLic-Nus pBAD-DEST49 |
| D_nodosus | 1 | 1218024 | 1218608 | ZZ | DNO_1171 | CDS | pal | DN1176 VC25 pDEST-17 pLic-Nus pBAD-DEST49 |
| D_nodosus | 1 | 1238072 | 1236759 | ZZ | DNO_1190 | CDS | | interest DN1194 VC169 pDEST-17 pLic-Nus pBAD- |
| | | | | | | | | DEST49 |
| D_nodosus | 1 | 1242646 | 1241243 | ZZ | DNO_1192 | CDS | | DN1196 VC26 pDEST-17 pLic-Nus pBAD-DEST49 |
| D_nodosus | 1 | 1283149 | 1284618 | ZZ | DNO_1241 | CDS | | DN1237 VC174 pDEST-17 pLic-Nus pBAD-DEST49 |
| D_nodosus | 1 | 1327812 | 1329248 | ZZ | DNO_1302 | CDS | | DN0010 VC27 pDEST-17 pLIC-Nus pBAD-DEST49 |
| D_nodosus | 1 | 1337830 | 1338666 | ZZ | DNO_1310 | CDS | comL | DN0013 VC28 pDEST-17 pLIC-Nus pBAD-DEST49 |
| D_nodosus | 1 | 1346829 | 1347515 | ZZ | DNO_1317 | CDS | dsbC | DN0020 VC56 pDEST-17 pLIC-Nus pBAD-DEST49 |
| D_nodosus | 1 | 1349062 | 1350333 | ZZ | DNO_1320 | CDS | | DN0023 VC57 pDEST-17 pLIC-Nus pBAD-DEST49 |
| D_nodosus | 1 | 1352918 | 1351854 | ZZ | DNO_1322 | CDS | | DN0026 pDEST-17 pLIC-Nus pBAD-DEST49 |
| D_nodosus | 1 | 1356754 | 1356161 | ZZ | DNO_1327 | CDS | | DN0031 VC59 pDEST-17 pLIC-Nus pBAD-DEST49 |
| D_nodosus | 1 | 1357841 | 1357317 | ZZ | DNO_1329 | CDS | | DNCD33 VC60 pDEST-17 pLIC-Nus pBAD-DEST49 |
| D_nodosus | 1 | 1369083 | 1366930 | ZZ | DNO_1338 | CDS | ptrB | DN0043 VC61 pLIC-Nus pBAD-DEST49 |
| D_nodosus | 1 | 1373146 | 1373895 | ZZ | DNO_1343 | CDS | | DN0048 VC62 pDEST-17 pLic-Nus pBAD-DEST49 |
| D_nodosus | 1 | 1375607 | 1374870 | ZZ | DNO_1345 | CDS | | DN0050 VC63 pDEST-17 pLic-Nus pBAD-DEST49 |
| D_nodosus | 1 | 1378439 | 1377828 | ZZ | DNO_1348 | CDS | | DN0053 VC001 pDEST-17 pLic-Nus pBAD-DEST49 |
| D_nodosus | 1 | 1384148 | 1381989 | ZZ | DNO_1351 | CDS | dcpA | DN0056 VC64 pDEST-17 pLIC-Nus pBAD-DEST49 |

### Example 8

### Field Immunization Trial

The ability of the recombinant antigens to protect against ovine footrot is examined in either a field virulence trial or a pen virulence trial.

A field trial of the vaccine is conducted on pasture which receives sufficient moisture for both pasture growth and transmission of footrot, if necessary using flood irrigation to supplement natural rainfall.

Merino sheep are vaccinated twice subcutaneously in the neck region, with an interval of 4 weeks between the injections. Any of the polypeptides of the invention may be used as the antigen. For example the antigen may be selected from the eight proteins identified in Example 5 as reacting with sera from sheep which have recovered from D. nodosus infection. It will be clearly understood that a combination of two or more of such antigens may be used, or that a combination of one or more of these antigens together with one or more previously-known protective antigens may be used.

Each protein is diluted in PBS to the required concentration. The amount of protein in each fraction is estimated by a modified Lowry method (Hartree 1972) or other suitable method. Many methods for protein estimation are known in the art. The aqueous phase of the vaccine is emulsified with incomplete Freund's adjuvant (Difco) in the ratio 1:2. Alternative adjuvants may be used, as discussed in Example 7.

Immunised and control groups of 2 to 10 sheep per group are used. The sheep are randomly allocated to each of the groups on a bodyweight basis. All groups are run as one flock, and are exposed at the time of the second vaccination to donor sheep previously infected with the virulent D. nodosus strain VCS1703A.

The sheep are immunised and bled at 55, 30 and 0 days prior to challenge. Sheep are injected subcutaneously behind the ear with 1 ml of vaccine. The control group is injected with saline alone. Each test sheep is injected with 50 to 1000 μg of a recombinant candidate antigen in saline.

On day 0 every foot of each sheep is subjected to experimental challenge with an agar culture of D. nodosus strain VCS1703A, the homologous virulent strain. The progression of disease on each foot is measured using a standard lesion scoring method (Whittington & Nicholls, 1995, Egerton & Roberts, 1971) at 14, 21, 27, 35, 42 and 49 days after challenge, in order to assess whether there is any significant protective effect after homologous challenge. The sheep are also bled at each of these time intervals, and the sera are tested for antibodies directed against the proteins used for immunization.

### Example 9

### Pen Immunization Trial

Pen immunization trials are performed using the method of Kennan et al. (2001). Six-month-old Merino sheep confirmed as being free of footrot are randomly allocated into groups of 2 to 10 sheep, and housed in an animal house on concrete floors, each group in a separate pen. The sheep are fed lucerne hay and oats; water is provided ad libitum. The groups of animals are immunised as described in Example 8.

The feet of the animals are then predisposed to infection by keeping the animals on wet foam mats for 4 days prior to challenge, to facilitate maceration of the skin. All animals are sampled for D. nodosus with a swab stick applied to the interdigital skin prior to challenge.

The sheep are then challenged by applying 4-day-old cultures of each strain, on plugs of 2% hoof agar (Thomas 1958), to the interdigital skin and holding them in place with bandages for 4 days. Each plug contains 8.4×10⁵ to 9.5×10⁵ CFU of D. nodosus; uninoculated agar is used for the negative control. The mats are removed from the floor one week after the start of the challenge, and the animals are again sampled for D. nodosus. All animals are examined, and their feet scored for footrot lesions at the start of the trial and then at weekly intervals. A standard lesion scoring method is used (Whittington & Nicholls, 1995, Egerton & Roberts, 1971). The total weighted foot score (TWFS), which includes information from each of the four feet, is used to provide an unambiguous overall score for the animal.

Further tests which may be performed are set out in the Australian Pesticides and Veterinary Medicines Authority's Guidelines for the Registration of Agents for the control and treatment of non-benign ovine footrot, which may be found at http://www.apvma.gov.au/guidelines/footrot.shtml.

### Example 10

### Sheep Immunization Trial

The ability of the recombinant antigens to protect against ovine footrot is examined in a field and pen-based virulence trial.

Merino sheep confirmed as being free of footrot are agisted on pasture and vaccinated twice subcutaneously in the neck region, with an interval of 30 days between the injections. Any of the polypeptides of the invention may be used as the antigen. For example, the antigen may be selected from the eight proteins identified in Example 5 as reacting with sera from sheep which have recovered from D. nodosus infection. It will be clearly understood that a combination of two or more of such antigens may be used, or that a combination of one or more of these antigens together with one or more previously-known protective antigens may be used.

Each protein is diluted in PBS to the required concentration. The amount of protein in each fraction is estimated by a conventional method, such as a modified Lowry method. Many methods for protein estimation are known in the art. The aqueous phase of the vaccine is emulsified with incomplete Freund's adjuvant (Difco) in the ratio 1:2. Alternative adjuvants may be used, as discussed in Example 7.

Immunised and control groups of 8 sheep per group are used. The sheep are tagged and randomly allocated to each of the groups on a bodyweight basis. All groups are initially run on pasture as one flock.

The sheep are bled at 65, 35 and 0 days prior to challenge. Sheep are injected subcutaneously behind the ear with 1 ml of vaccine at 65 and 35 days prior to challenge. The control group is injected with saline alone. Each test sheep is injected with 50 to 1000 μg of a recombinant candidate antigen in saline.

At 10 days prior to challenge the sheep are brought off the pasture and placed into separate enclosed pens in a large barn-like facility. Each group of eight sheep occupies a separate pen. The sheep are fed lucerne hay and oats; water is provided ad libitum. Five days prior to challenge the sheep are placed on wet foam mats, to facilitate maceration of the skin and to simulate the wet conditions normally required for footrot transmission. The feet of the animals are thus predisposed to infection. All animals are sampled for D. nodosus with a swab stick applied to the interdigital skin prior to challenge. On day 0 every foot of each sheep is subjected to experimental challenge (Kennan et al. (2001)) by applying 4-day-old cultures of D. nodosus strain VCS1703A on plugs of 2% hoof agar (Thomas 1958) to the interdigital skin and holding them in place with bandages for 4 days. Each plug contains 8.4×10⁵ to 9.5×10⁵ CFU of D. nodosus; uninoculated agar is used for the negative control. The mats are removed from the floor one week after the start of the challenge, and the animals are again sampled for D. nodosus.

The progression of disease on each foot is measured using a standard lesion scoring method (Whittington & Nicholls, 1995, Egerton & Roberts, 1971) at 0, 14, 21, 27, and 35 days after challenge, in order to assess whether there is any significant protective effect after homologous challenge. The total weighted foot score (TWFS), which includes information from each of the four feet, is used to provide an unambiguous overall score for the extent of disease in each animal. The TWFS from the test groups are compared to the TWFS from the control group to determine whether immunization has protected the animals from disease. The sheep are also bled at each of these time intervals, and the sera are tested for antibodies directed against the proteins used for immunization.

Two trials using this protocol have commenced, and the antigens used for the injection were as follows:

Vaccine Trial 1

| | | |
|---|---|---|
| 01) | DN0_0012 | (SEQ ID NO: 179) |
| 02) | DN0_0033 | (SEQ ID NO: 181) |
| 03) | DN0_0603 | (SEQ ID NO: 183) |
| 04) | DN0_0605 | (SEQ ID NO: 184) |
| 05) | DN0_0644 | (SEQ ID NO: 185) |
| 06) | DN0_0725 | (SEQ ID NO: 187) |
| 07) | DN0_1167 | (SEQ ID NO: 189) |
| 08) | DN0_1241 | (SEQ ID NO: 190) |
| 09) | DN0_0320 | |
| 10) | DN0_0081 | |
| 11) | DN0_0494 | |
| 12) | DN0_0861 | |
| 13) | DN0_0110 | |

Vaccine Trial 2

| | |
|---|---|
| 14) | DN0_1348 |
| 15) | DN0_0650 |
| 16) | DN0_0890 |
| 17) | DN0_0700 |
| 18) | DN0_1170 |
| 19) | DN0_1302 |
| 20) | DN0_1343 |
| 21) | DN0_0024 |
| 22) | DN0_0424 |
| 23) | DN0_1192 |
| 24) | DN0_0519 |
| 25) | DN0_0873 |
| 26) | DN0_0316 |

In subsequent trials the remaining proteins listed in Table 8 are used for immunization.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

References cited herein are listed on the following pages, and are incorporated herein by this reference.

### REFERENCES

Billington, S. J., Johnston, J. L. & Rood, J. I. Virulence regions and virulence factors of the ovine footrot pathogen, Dichelobacter nodosus. FEMS Microbiol. Lett. 145, 147-156 (1996).
Bothelo, E. et al. Endocarditis caused by Cardiobacterium valvarum. J Clin Microbiol 44, 657-658 (2006).
Bulach, D. M. et al. Genome reduction in Leptospira borgpetersenii reflects limited transmission potential. Proceedings of the National Academy of Sciences U.S.A (2006).
Cabrita, L. D., Dai, W. & Bottomley, S. P. A family of E. coli expression vectors for laboratory scale and high throughput soluble protein production. BMC Biotechnology 6, 12 (2006).
Calza, L., Manfredi, R. & Chiodo, F. Infective endocarditis: a review of the best treatment options. Expert Opin Pharmacother 5, 1899-1916 (2004).
Chou, P. Y. & Fasman, G. D. 1974a Conformational parameters for amino acids in helical, β-sheet, and random coil regions calculated from proteins. Biochemistry, 13, 211-222 (1974).
Chou, P. Y. & Fasman, G. D. 1974b Prediction of protein Conformation. Biochemistry, 13, 222-245 (1974).
Cianciotto, N. P. & Fields, B. S. Legionella pneumophila mip gene potentiates intracellular infection of protozoa and human macrophages. Proceedings of the National Academy of Sciences U.S.A 89, 5188-5191 (1992).
Claxton, P. D., Riberiro, L. A. & Egerton, J. R. Classification of Bacteroides nodosus by agglutination tests. Aust. Vet. J. 60, 331-334 (1983).
Delcher, A. L., Phillippy, A., Carlton, J. & Salzberg, S. L. Fast algorithms for large-scale genome alignment and comparison. Nucleic Acids Res 30, 2478-2483 (2002). Gish, W. 2004).
Edgar, R. C. MUSCLE: multiple sequence alignment with high accuracy and high throughput. Nucleic Acids Res 32, 1792-1797 (2004).
Egerton, J. R. & Burrell, D. H. Prophylactic and therapeutic vaccination against ovine foot-rot. Aust. Vet. J. 46, 517-522 (1970).
Egerton, J. R., and D. S. Roberts. 1971. Vaccination against ovine foot-rot. J. Comp. Pathol. 81:179-185.
Foster, G. et al. Suttonella ornithocola sp. nov., from birds of the tit families, and emended description of the genus Suttonella. Int J Syst Evol Microbiol 55, 2269-2272 (2005).
Fraser, C. M. et al. Genomic sequence of a Lyme disease spirochaete, Borrelia burgdorferi. Nature 390, 580-586 (1997).
Frey, J. & Kuhnert, P. RTX toxins in Pasteurellaceae. Int J Med Microbiol 292, 149-158 (2002).
Gardy, J. L. et al. PSORT-B: Improving protein subcellular localization prediction for Gram-negative bacteria. Nucleic Acids Research 31, 3613-3617 (2003).
Ghimire, S. C., Egerton, J. R., Dhungyel, O. P. & Joshi, H. D. Identification and characterisation of serogroup M among Nepalese isolates of Dichelobacter nodosus, the transmitting agent of footrot in small ruminants. Veterinary Microbiology 62, 217-233. (1998).
Giovannoni, S. J. et al. Genome streamlining in a cosmopolitan oceanic bacterium. Science 309, 1242-1245 (2005).
Haring, V. et al. Delineation of the virulence-related locus (vrl) of Dichelobacter nodosus. Microbiology 141, 2081-2089 (1995).
Hess, J. et al. Listeria monocytogenes p60 supports host cell invasion by and in vivo survival of attenuated Salmonella typhimurium. Infection and Immunity 63, 2047-2053 (1995).
Hobbs, M. et al. Organization of the fimbrial gene region of Bacteroides nodosus: class I and class II strains. Mol. Microbiol. 5, 543-560 (1991).
Hopp T. P. & Woods K. R. Prediction of protein antigenic determinants from amino acid sequences. Proc Natl Acad Sci USA. 78:3824-8 (1981)
Holzem et al: Rapid identification of a tobacco mosaic virus epitope by using a coat protein gene-fragment-pVIII fusion library. Journal of General Virology. 82, 9-15 (2001)
Howe, K., Bateman, A. & Durbin, R. QuickTree: building huge Neighbour-Joining trees of protein sequences. Bioinformatics 18, 1546-1547 (2002).
Kennan, R. M., Billington, S. J. & Rood, J. I. Electroporation-mediated transformation of the ovine footrot pathogen Dichelobacter nodosus. FEMS Microbiol Lett 169, 383-389 (1998).
Kennan, R. M., Dhungyel, O. P., Whittington, R. J., Egerton, J. R. & Rood, J. I. The Type IV fimbrial subunit gene (fimA) of Dichelobacter nodosus is essential for virulence, protease secretion, and natural competence. Journal of Bacteriology 183, 4451-4458 (2001).
Kennan, R. M., Dhungyel, O. P., Whittington, R. J., Egerton, J. R. & Rood, J. I. Transformation-mediated serogroup conversion of Dichelobacter nodosus. Veterinary Microbiology 92, 169-178 (2003).
Kirkwood, J. K., Macgregor, S. K., Malnick, H. & Foster, G. Unusual mortality incidents in tit species (family Paridae) associated with the novel bacterium Suttonella ornithocola. Vet Rec 158, 203-205 (2006).
Konstantinidis, K. T. & Tiedje, J. M. Trends between gene content and genome size in prokaryotic species with larger genomes. Proceedings of the National Academy of Sciences U.S.A 101, 3160-3165 (2004).
Kuhn, M. & Goebel, W. Identification of an extracellular protein of Listeria monocytogenes possibly involved in intracellular uptake by mammalian cells. Infection and Immunity 57, 55-61 (1989).
Leuzzi, R. et al. Ng-MIP, a surface-exposed lipoprotein of Neisseria gonorrhoeae, has a peptidyl-prolyl cis/trans isomerase (PPIase) activity and is involved in persistence in macrophages. Mol. Microbiol. 58, 669-681 (2005).
Loosmore, S. M. et al. Outer membrane protein D15 is conserved among Haemophilus influenzae species and may represent a universal protective antigen against invasive disease. Infection and Immunity 65, 3701-3707 (1997).
Lu, Z. et al. Predicting subcellular localization of proteins using machine-learned classifiers. Bioinformatics 20, 547-556 (2004).
Mac Siomoin, R. A. et al. Identification and characterization of ispA, a Shigella flexneri chromosomal gene essential for normal in vivo cell division and intracellular spreading. Mol. Microbiol. 19, 599-609 (1996).
Moran, N. A. Microbial minimalism: genome reduction in bacterial pathogens. Cell 108, 583-586 (2002).
Moses, E. K. et al. A multiple site-specific DNA-inversion model for the control of Omp1 phase and antigenic variation in Dichelobacter nodosus. Mol Microbiol 17, 183-196 (1995).
Nielsen, H., Engelbrecht, J., Brunak, S. & von Heijne, G. Identification of prokaryotic and eukaryotic signal peptides and prediction of their cleavage sites. Protein Engineering 10, 1-6 (1997).
Nunn, D. Bacterial type II protein export and pilus biogenesis: more than just homologies? Trends in Cell Biology 9, 402-408. (1999).
Parker, D. et al. Regulation of type IV fimbrial biogenesis in Dichelobacter nodosus. J Bacteriol 188, 4801-4811 (2006).
Parker, D., Kennan, R. M., Myers, G. S., Paulsen, I. & Rood, J. I. Identification of a Dichelobacter nodosus ferric uptake regulator and determination of its regulatory targets. Journal of Bacteriology 187, 366-375 (2005).
Perez-Rueda, E. & Collado-Vides, J. The repertoire of DNA-binding transcriptional regulators in Escherichia coli K-12. Nucleic Acids Research 28, 1838-1847 (2000).
Pizza, M. et al. Identification of vaccine candidates against serogroup B meningococcus by whole-genome sequencing. Science 287, 1816-1820 (2000).
Riffkin, M. C., Wang, L. F., Kortt, A. A. & Stewart, D. J. A single amino-acid change between the antigenically different extracellular serine proteases V2 and B2 from Dichelobacter nodosus. Gene 167, 279-283 (1995).
Rood, J. I. Genomic islands of Dichelobacter nodosus. Current Topics in Microbiology and Immunology 264, 47-60 (2002).
Rood, J. I., Stewart, D. J., Vaughan, J. A. & Dewhirst, F. E. in Bergy's manual of systematic bacteriology, Volume 2: The Proteobacteria; Part B: The gammaproteobacteria, Edn. 2nd. (eds. D. J. Brenner, N. R. Kreig & J. T. Staley) 124-129 (Springer, New York; 2005).
Ruffolo, C. G. & Adler, B. Cloning, sequencing, expression, and protective capacity of the oma87 gene encoding the Pasteurella multocida 87-kilodalton outer membrane antigen. Infection and Immunity 64, 3161-3167 (1996).
Sallstrom, B. & Andersson, S. G. Genome reduction in the alpha-Proteobacteria. Curr Opin Microbiol 8, 579-585 (2005).
Sandkvist, M. Biology of type II secretion. Mol. Microbiol. 40, 271-283. (2001).
Schwartzkoff, C. L. et al. The effects of antigenic competition on the efficacy of multivalent footrot vaccines. Aust Vet J 70, 123-126 (1993).
Stewart, D. J. in Footrot and foot abscess of ruminants. (eds. J. R. Egerton, W. K. Yong & G. G. Riffkin) 5-45 (CRC Press, Boca Raton; 1989).
Stewart, D. J., Clark, B. L., Emery, D. L., Peterson, J. E. & Fahey, K. J. A Bacteroides nodosus immunogen, distinct from the pilus, which induces cross-protective immunity in sheep vaccinated against footrot. Aust. Vet. J. 60, 83-85 (1983).
Stewart, D. J. et al. The protection given by pilus and whole cell vaccines of Bacteroides nodosus strain 198 against ovine foot-rot induced by strains of different serogroups. Aust. Vet. J. 62, 153-159 (1985).
Stover, C. K. et al. Complete genome sequence of Pseudomonas aeruginosa PA01, an opportunistic pathogen. Nature 406, 959-964 (2000).
Suzuki, T. et al. Identification and characterization of a chromosomal virulence gene, vacJ, required for intercellular spreading of Shigella flexneri. Mol. Microbiol. 11, 31-41 (1994).
Tettelin, H., Radune, D., Kasif, S., Khouri, H. & Salzberg, S. L. Optimized multiplex PCR: efficiently closing a whole-genome shotgun sequencing project. Genomics 62, 500-507 (1999).
Thomas, J. H. A Simple Medium for the Isolation and Cultivation of Fusiformis nodosus. Aust. Vet. J., 411 (1958).
Thomas, J. H. The pathogenesis of footrot in sheep with reference to proteases of Fusiformis nodosus. Aust. J. Agric. Res 15, 1001-1016 (1964).
Thorley, C. M. & Egerton, J. R. Comparison of alum-absorbed or non-alum-absorbed oil emulsion vaccines containing either pilate or non pilate Bacteroides nodosus cells in inducing and maintaining resistance of sheep to experimental footrot. Res. Vet. Sci. 30:32-37 (1981)
Vissa, V. D. & Brennan, P. J. The genome of Mycobacterium leprae: a minimal mycobacterial gene set. Genome Biol 2, REVIEWS1023 (2001).
Whittington, R. J., and P. J. Nicholls. 1995. Grading the lesions of ovine footrot. Res. Vet. Sci. 58:26-34.
Yorgey, P., Rahme, L. G., Tan, M. W. & Ausubel, F. M. The roles of mucD and alginate in the virulence of Pseudomonas aeruginosa in plants, nematodes and mice. Mol. Microbiol. 41, 1063-1076 (2001).
Zhou, H. & Hickford, J. G. Extensive diversity in New Zealand Dichelobacter nodosus strains from infected sheep and goats. Veterinary Microbiology 71, 113-123 (2000).

1-35. (canceled)

36. An isolated immunogenic Dichelobacter nodosus polypeptide, selected from the group consisting of PilT, PilU, ChpA, PilJ, PilI, PilG, PilH, Ppk and FimX, and the proteins encoded by the gene loci listed in Table 3, Table 5 or Table 6, or a biologically active or immunogenic fragment, derivative, or variant thereof, with the proviso that the polypeptide is not AprV2 (DNO_{—}1167; SEQ ID NO: 189), AprV5 (DNO_{—}0603; SEQ ID NO: 183), BprV (DNO_{—}0605; SEQ ID NO: 184), YfeA (DNO_{—}0644; SEQ ID NO: 185), or a fragment or variant thereof.

37. A polypeptide according to claim 36, selected from the group consisting of the proteins encoded by the gene loci listed in Table 3, Table 5 or Table 6.

38. A polypeptide according to claim 36, which is selected from the group consisting of:
a) a protein involved in transport of molecules across a Dichelobacter nodosus outer membrane;
b) a protein involved in iron uptake;
c) a protein involved in transport of nutrients;
d) a protein involved in resistance of Dichelobacter nodosus to heavy metals; and
e) a protein involved in synthesis or structural stability of the outer membrane.

39. A polypeptide according to claim 36, which is selected from the group consisting of DNO_{—}0012 (SEQ ID NO: 179), DNO_{—}0033 (SEQ ID NO: 181), DNO_{—}0725 (SEQ ID NO: 187) and DNO_{—}1241 (SEQ ID NO: 190).

40. An isolated antibody which specifically binds to a Dichelobacter nodosus polypeptide according to claim 36, or to a biologically-active or immunogenic fragment, derivative, or variant thereof.

41. An isolated nucleic acid molecule which encodes a polypeptide according to claim 36.

42. A composition or kit comprising a polypeptide according to claim 36, together with a pharmaceutically- or veterinarily-acceptable carrier.

43. A vaccine capable of treating or preventing a disease caused by D. nodosus, comprising one or more surface-exposed or secreted polypeptides of D. nodosus or biologically active or immunogenic fragments, derivatives or variants thereof, the one or more polypeptides comprising signal and lipoprotein peptides and having fewer than 2 transmembrane domains, and being reactive against sera recovered from animals repeatedly infected with D. nodosus, with the proviso that if individual polypeptides are used, then they are not AprV2 (DNO_{—}1167; SEQ ID NO: 189), AprV5 (DNO_{—}0603; SEQ ID NO: 183), BprV (DNO_{—}0605; SEQ ID NO: 184), a polypeptide encoded by the Omp1 locus, or a fimbrial polypeptide encoded by the fimA fimbrial subunit gene.

44. A vaccine capable of treating or preventing a disease caused by D. nodosus, comprising one or more surface-exposed or secretes polypeptides of D. nodosus or a biologically active or immunogenic fragment, derivative or variant thereof, comprising
(a) one or more isolated immunogenic D. nodosus polypeptides according to claim 36,
(b) one or more of YfeA (DNO_{—}0644; SEQ ID NO: 185), AprV2 (DNO_{—}1167; SEQ ID NO: 189), AprV5 (DNO_{—}0603; SEQ ID NO: 183), BprV (DNO_{—}0605; SEQ ID NO: 184), a polypeptide encoded by the Omp1 locus, and/or a fimbrial polypeptide encoded by the fimA fimbrial subunit gene, or
an immunogenic fragment, derivative, or variant thereof,
with the proviso that if the vaccine comprises AprV2 (DNO_{—}1167; SEQ ID NO: 189), AprV5 (DNO_{—}0603; SEQ ID NO: 183), BprV (DNO_{—}0605; SEQ ID NO: 184), a polypeptide encoded by the Omp1 locus, and/or a fimbrial polypeptide encoded by the fimA fimbrial subunit gene, or an immunogenic fragment, derivative, or variant thereof, at least one of (a) or YfeA (DNO_{—}0644; SEQ ID NO: 185) is present.

45. A composition or kit comprising a nucleic acid molecule according to claim 41, together with a pharmaceutically or veterinarily acceptable carrier.

46. A composition or kit comprising an antibody according to claim 40, together with a pharmaceutically or veterinarily acceptable carrier.

47. A kit for detecting the presence of DNA associated with Dichelobacter nodosus in a sample, in which the kit comprises:
a) a known amount of a first oligonucleotide which consists of at least about 7 to about 50 nucleotides, in which the oligonucleotide has at least about 70% contiguous sequence identity to a nucleic acid molecule which encodes a polypeptide according to claim 36;
b) a known amount of a second oligonucleotide, in which the second oligonucleotide consists of at least about 7 to about 50 nucleotides, and in which the oligonucleotide has at least about 70% contiguous sequence identity to a nucleotide sequence which is complementary to a nucleic acid molecule which encodes a polypeptide according to claim 36, and optionally
c) reagents for nucleic acid amplification.

48. A method of detecting a nucleic acid molecule encoding a polypeptide of Dichelobacter nodosus, comprising the step of contacting a nucleic acid obtained from a biological sample of a subject with at least two oligonucleotides, under conditions effective to amplify the nucleic acid so as to yield an amount of amplified nucleic acid, in which
a) at least one of the oligonucleotides is specific for a nucleic acid according to claim 41, and
b) the biological sample comprises cells suspected of containing a nucleic acid molecule encoding the immunogenic polypeptide.

49. A library of candidate immunogenic Dichelobacter nodosus polypeptides, comprising the polypeptides encoded by the gene loci listed in Table 3, Table 5 or Table 6, PilT, PilU, ChpA, PilJ, PilI, PilG, PilH, Ppk and FimX.

50. A library of nucleic acid molecules encoding immunogenic Dichelobacter nodosus polypeptides, comprising nucleic acid molecules encoding the polypeptides encoded by the gene loci listed in Table 3, Table 5 or Table 6, PilT, PilU, ChpA, PilJ, PilI, PilG, PilH, Ppk and FimX.

51. A method of screening for a candidate immunogenic D. nodosus polypeptide, comprising testing a polypeptide library according to claim 49, expressed from the nucleic acid library encoding immunogenic Dichelobacter nodosus polypeptides, comprising nucleic acid molecules encoding the polypeptides according to claim 49, for the ability to react with antibodies present in sera of animals previously exposed to D. nodosus infection or to elicit protective antibodies against D. nodosus.

52. A method of screening for a candidate immunogenic D. nodosus polypeptide comprising testing a polypeptide expressed from the nucleic acid library according to claim 50, for the ability to react with antibodies present in sera of animals previously exposed to D. nodosus infection or to elicit protective antibodies against D. nodosus.

53. A method of treating or preventing a disease or condition caused by Dichelobacter nodosus, comprising the step of administering a polypeptide according to claim 36, to a subject suffering from, suspected to be suffering from or at risk of such a disease or condition.

54. A method of treating or preventing a disease or condition caused by Dichelobacter nodosus, comprising the step of administering a antibody according to claim 40, to a subject suffering from, suspected to be suffering from or at risk of such a disease or condition.

55. A method of treating or preventing a disease or condition caused by Dichelobacter nodosus, comprising the step of administering a nucleic acid molecule according to claim 41, to a subject suffering from, suspected to be suffering from or at risk of such a disease or condition.

56. A method of treating or preventing a disease or condition caused by Dichelobacter nodosus, comprising the step of administering a vaccine according to claim 43, to a subject suffering from, suspected to be suffering from or at risk of such a disease or condition.

57. A method of diagnosing a disease or condition caused by Dichelobacter nodosus, comprising the step of detecting a polypeptide according to claim 36 in a biological sample from a subject suffering from, suspected to be suffering from, or at risk of such a disease or condition.

58. A method of diagnosing a disease or condition caused by Dichelobacter nodosus, comprising the step of detecting a antibody according to claim 40 in a biological sample from a subject suffering from, suspected to be suffering from, or at risk of such a disease or condition.

59. A method of diagnosing a disease or condition caused by Dichelobacter nodosus, comprising the step of detecting a nucleic acid molecule according to claim 41 in a biological sample from a subject suffering from, suspected to be suffering from, or at risk of such a disease or condition.


SEQUENCE LISTING (201 sequences)

SEQ ID NO: 1 - DNA, 30 bp, Artificial
  Synthetic oligonucleotide
caccatgagtcataaaacaaagcacaaatc

SEQ ID NO: 2 - DNA, 20 bp, Artificial
  Synthetic oligonucleotide
attctcatcatctggcgatg

SEQ ID NO: 3 - DNA, 29 bp, Artificial
  Synthetic oligonucleotide
caccatgggcgttgtgttaaaaaatgaag

SEQ ID NO: 4 - DNA, 20 bp, Artificial
  Synthetic oligonucleotide
ttttttagccgcttcatcag

SEQ ID NO: 5 - DNA, 30 bp, Artificial
  Synthetic oligonucleotide
caccatgagcgtagatatcaacattttagc

SEQ ID NO: 6 - DNA, 22 bp, Artificial
  Synthetic oligonucleotide
taacggcgttgataaacgcgac

SEQ ID NO: 7 - DNA, 26 bp, Artificial
  Synthetic oligonucleotide
caccatggtgcaaagcgacagcatca

SEQ ID NO: 8 - DNA, 23 bp, Artificial
  Synthetic oligonucleotide
acgggtaatacgaataccacttc

SEQ ID NO: 9 - DNA, 27 bp, Artificial
  Synthetic oligonucleotide
caccatggcggatatgacgattaaacc

SEQ ID NO: 10 - DNA, 27 bp, Artificial
  Synthetic oligonucleotide
aaacgttttagaaaattccacaaccac

SEQ ID NO: 11 - DNA, 27 bp, Artificial
  Synthetic oligonucleotide
caccatggcggataaatatcgcgttgc

SEQ ID NO: 12 - DNA, 22 bp, Artificial
  Synthetic oligonucleotide
attagcggattccatttccaac

SEQ ID NO: 13 - DNA, 28 bp, Artificial
  Synthetic oligonucleotide
caccatggaaacttatcaggttggcacc

SEQ ID NO: 14 - DNA, 22 bp, Artificial
  Synthetic oligonucleotide
attagcggattccatttccaac

SEQ ID NO: 15 - DNA, 27 bp, Artificial
  Synthetic oligonucleotide
caccatgcaagaaatcgcccagcagta

SEQ ID NO: 16 - DNA, 21 bp, Artificial
  Synthetic oligonucleotide
tcggcggcaatcagaagcaag

SEQ ID NO: 17 - DNA, 27 bp, Artificial
  Synthetic oligonucleotide
caccatgagcagcaccaatcccagcca

SEQ ID NO: 18 - DNA, 25 bp, Artificial
  Synthetic oligonucleotide
tttaatcgcattaatatcttggata

SEQ ID NO: 19 - DNA, 29 bp, Artificial
  Synthetic oligonucleotide
caccatgctgcaaaatgtggaaaaatcac

SEQ ID NO: 20 - DNA, 28 bp, Artificial
  Synthetic oligonucleotide
gttgagttcaaaacgttttcctatgtta

SEQ ID NO: 21 - DNA, 29 bp, Artificial
  Synthetic oligonucleotide
caccatggtttctaacggtggcggtttga

SEQ ID NO: 22 - DNA, 22 bp, Artificial
  Synthetic oligonucleotide
ataaacagattgataagccgcc

SEQ ID NO: 23 - DNA, 27 bp, Artificial
  Synthetic oligonucleotide
caccatgcaagaagaaaaagcgccggc

SEQ ID NO: 24 - DNA, 20 bp, Artificial
  Synthetic oligonucleotide
ttcggtagcgatgcccattg

SEQ ID NO: 25 - DNA, 28 bp, Artificial
  Synthetic oligonucleotide
caccatgatccctgcatacaacgactac

SEQ ID NO: 26 - DNA, 26 bp, Artificial
  Synthetic oligonucleotide
tggagtagcttctttcaatgctttag

SEQ ID NO: 27 - DNA, 26 bp, Artificial
  Synthetic oligonucleotide
caccatggacaccatcatcacccgtt

SEQ ID NO: 28 - DNA, 22 bp, Artificial
  Synthetic oligonucleotide
tgatgcccgatgtttatcggga

SEQ ID NO: 29 - DNA, 26 bp, Artificial
  Synthetic oligonucleotide
caccatgcgcgcacccgaacaaaacg

SEQ ID NO: 30 - DNA, 27 bp, Artificial
  Synthetic oligonucleotide
atagacactcaaaatgtgatttaattc

SEQ ID NO: 31 - DNA, 30 bp, Artificial
  Synthetic oligonucleotide
caccatgcattttcaattaatttatacgcc

SEQ ID NO: 32 - DNA, 27 bp, Artificial
  Synthetic oligonucleotide
caatgattctgttgccaattcctcagc

SEQ ID NO: 33 - DNA, 29 bp, Artificial
  Synthetic oligonucleotide
caccatgtcttcaacgaaagacccctttc

SEQ ID NO: 34 - DNA, 20 bp, Artificial
  Synthetic oligonucleotide
cggacggacaatccagtctg

SEQ ID NO: 35 - DNA, 25 bp, Artificial
  Synthetic oligonucleotide
caccatgcaaagcggcaatgtgatg

SEQ ID NO: 36 - DNA, 20 bp, Artificial
  Synthetic oligonucleotide
taaagcggcaggaccagaac

SEQ ID NO: 37 - DNA, 31 bp, Artificial
  Synthetic oligonucleotide
caccatgatgttgatttgcagtttgaagaag

SEQ ID NO: 38 - DNA, 27 bp, Artificial
  Synthetic oligonucleotide
tacaatgtaaatgcttgtttgatcgat

SEQ ID NO: 39 - DNA, 27 bp, Artificial
  Synthetic oligonucleotide
caccatgggttttcatctgcgcgcgac

SEQ ID NO: 40 - DNA, 22 bp, Artificial
  Synthetic oligonucleotide
ttgcggtaattgctgttgtttc

SEQ ID NO: 41 - DNA, 30 bp, Artificial
  Synthetic oligonucleotide
caccatggataacgctaacaatcaatccgc

SEQ ID NO: 42 - DNA, 21 bp, Artificial
  Synthetic oligonucleotide
ttttttagcgtttatttcatc

SEQ ID NO: 43 - DNA, 32 bp, Artificial
  Synthetic oligonucleotide
caccatggaaacgtataaagttgttattgaac

SEQ ID NO: 44 - DNA, 28 bp, Artificial
  Synthetic oligonucleotide
aaaataaggttttttccctacaatattg

SEQ ID NO: 45 - DNA, 28 bp, Artificial
  Synthetic oligonucleotide
caccatgaaagagccgctgcatttaatg

SEQ ID NO: 46 - DNA, 19 bp, Artificial
  Synthetic oligonucleotide
cggttgcacggcatactcc

SEQ ID NO: 47 - DNA, 29 bp, Artificial
  Synthetic oligonucleotide
caccatgattgaatcgccggtgatgatta

SEQ ID NO: 48 - DNA, 30 bp, Artificial
  Synthetic oligonucleotide
ttcatcagttgatgattctgttttttctgc

SEQ ID NO: 49 - DNA, 26 bp, Artificial
  Synthetic oligonucleotide
caccatgatggacgccagtcagcaac

SEQ ID NO: 50 - DNA, 21 bp, Artificial
  Synthetic oligonucleotide
ataaccgtaaccgccgccgta

SEQ ID NO: 51 - DNA, 27 bp, Artificial
  Synthetic oligonucleotide
caccatggctgaaattacggatatccg

SEQ ID NO: 52 - DNA, 26 bp, Artificial
  Synthetic oligonucleotide
ttgttctaaaagcatatgttgcggta

SEQ ID NO: 53 - DNA, 27 bp, Artificial
  Synthetic oligonucleotide
caccatgattgatagcgcgcctgccgt

SEQ ID NO: 54 - DNA, 27 bp, Artificial
  Synthetic oligonucleotide
tttttctggttgataataagtcggttc

SEQ ID NO: 55 - DNA, 27 bp, Artificial
  Synthetic oligonucleotide
caccatggaagaagaaactacggcagc

SEQ ID NO: 56 - DNA, 27 bp, Artificial
  Synthetic oligonucleotide
ctcgatttttaaagaacgtaacgtttc

SEQ ID NO: 57 - DNA, 30 bp, Artificial
  Synthetic oligonucleotide
caccatggatactatggaaaacctaacttc

SEQ ID NO: 58 - DNA, 23 bp, Artificial
  Synthetic oligonucleotide
gttaataacgcgttgaacagggc

SEQ ID NO: 59 - DNA, 26 bp, Artificial
  Synthetic oligonucleotide
caccatgcaagatagcgatgacggcg

SEQ ID NO: 60 - DNA, 23 bp, Artificial
  Synthetic oligonucleotide
attctgaaaaacggcaataacgc

SEQ ID NO: 61 - DNA, 30 bp, Artificial
  Synthetic oligonucleotide
caccatgggaacgatcacagggattgatgc

SEQ ID NO: 62 - DNA, 20 bp, Artificial
  Synthetic oligonucleotide
gttgtcgcgcaaattagaga

SEQ ID NO: 63 - DNA, 28 bp, Artificial
  Synthetic oligonucleotide
caccatgagcgatgaagcggaaaaaatc

SEQ ID NO: 64 - DNA, 26 bp, Artificial
  Synthetic oligonucleotide
ttttgaaacgtcaataatacaactgc

SEQ ID NO: 65 - DNA, 30 bp, Artificial
  Synthetic oligonucleotide
caccatgaaaacttatttggtcggtacgga

SEQ ID NO: 66 - DNA, 23 bp, Artificial
  Synthetic oligonucleotide
tcgccaacgcgcaataatttgac

SEQ ID NO: 67 - DNA, 32 bp, Artificial
  Synthetic oligonucleotide
caccatgcttcctagtttgaatatttacgcca

SEQ ID NO: 68 - DNA, 22 bp, Artificial
  Synthetic oligonucleotide
ctgaaaacgcgcgcgaattgct

SEQ ID NO: 69 - DNA, 31 bp, Artificial
  Synthetic oligonucleotide
caccatgcattgggatttcaatgaaaaaaac

SEQ ID NO: 70 - DNA, 18 bp, Artificial
  Synthetic oligonucleotide
tttctttttagcgctggt

SEQ ID NO: 71 - DNA, 30 bp, Artificial
  Synthetic oligonucleotide
caccatggtcgcgcgcacgatttggtattg

SEQ ID NO: 72 - DNA, 28 bp, Artificial
  Synthetic oligonucleotide
attaacggattgttgttctcgataaatc

SEQ ID NO: 73 - DNA, 27 bp, Artificial
  Synthetic oligonucleotide
caccatgcaaccgcgagcaattgctac

SEQ ID NO: 74 - DNA, 27 bp, Artificial
  Synthetic oligonucleotide
attaataaaataaacttctttcaacgc

SEQ ID NO: 75 - DNA, 26 bp, Artificial
  Synthetic oligonucleotide
caccatggaaacgctgcgccgcgcga

SEQ ID NO: 76 - DNA, 28 bp, Artificial
  Synthetic oligonucleotide
acgctcaatccataaatatttactatgc

SEQ ID NO: 77 - DNA, 30 bp, Artificial
  Synthetic oligonucleotide
caccatgggcgaaaaagatgcgcagtatta

SEQ ID NO: 78 - DNA, 20 bp, Artificial
  Synthetic oligonucleotide
tttaccgaattttttgagcg

SEQ ID NO: 79 - DNA, 29 bp, Artificial
  Synthetic oligonucleotide
caccatgcaattgtttcaaacggcagagc

SEQ ID NO: 80 - DNA, 21 bp, Artificial
  Synthetic oligonucleotide
atgttcgccgccgaccacatc

SEQ ID NO: 81 - DNA, 31 bp, Artificial
  Synthetic oligonucleotide
caccatggaaaataccacaaccctttatggc

SEQ ID NO: 82 - DNA, 21 bp, Artificial
  Synthetic oligonucleotide
gaaatcgtgacgcaatcctac

SEQ ID NO: 83 - DNA, 29 bp, Artificial
  Synthetic oligonucleotide
caccatggaaaacacaacaactctttatg

SEQ ID NO: 84 - DNA, 22 bp, Artificial
  Synthetic oligonucleotide
gaaatcgtgacgcaatcctaag

SEQ ID NO: 85 - DNA, 27 bp, Artificial
  Synthetic oligonucleotide
caccatggcaacacaaaaaacggatta

SEQ ID NO: 86 - DNA, 25 bp, Artificial
  Synthetic oligonucleotide
tggcaaaaatgattttaaagattgc

SEQ ID NO: 87 - DNA, 29 bp, Artificial
  Synthetic oligonucleotide
caccatggtgtcgacgacgcaatcaacgc

SEQ ID NO: 88 - DNA, 26 bp, Artificial
  Synthetic oligonucleotide
tattcctaaagattttgcttgaggtg

SEQ ID NO: 89 - DNA, 30 bp, Artificial
  Synthetic oligonucleotide
caccatgtcaagttggttttatggtcattc

SEQ ID NO: 90 - DNA, 20 bp, Artificial
  Synthetic oligonucleotide
ttgcggtcgaaaagcgacca

SEQ ID NO: 91 - DNA, 29 bp, Artificial
  Synthetic oligonucleotide
caccatggcggatctgccacagattctgc

SEQ ID NO: 92 - DNA, 23 bp, Artificial
  Synthetic oligonucleotide
gggtttttccggcaaagtatagc

SEQ ID NO: 93 - DNA, 27 bp, Artificial
  Synthetic oligonucleotide
caccatgtcttcaacgtattacgacgc

SEQ ID NO: 94 - DNA, 22 bp, Artificial
  Synthetic oligonucleotide
tgatgccgctcgcttcattttg

SEQ ID NO: 95 - DNA, 29 bp, Artificial
  Synthetic oligonucleotide
caccatgacgggcatgatgtatggcaatc

SEQ ID NO: 96 - DNA, 22 bp, Artificial
  Synthetic oligonucleotide
gaatgacgccgcgcgttcggca

SEQ ID NO: 97 - DNA, 27 bp, Artificial
  Synthetic oligonucleotide
caccatggccgtaaattacgaatccgc

SEQ ID NO: 98 - DNA, 20 bp, Artificial
  Synthetic oligonucleotide
gaatgcaacactccaacctc

SEQ ID NO: 99 - DNA, 30 bp, Artificial
  Synthetic oligonucleotide
caccatggctgaaagtatcgtcaactatga

SEQ ID NO: 100 - DNA, 22 bp, Artificial
  Synthetic oligonucleotide
gaatgtcaaactccaactatcg

SEQ ID NO: 101 - DNA, 27 bp, Artificial
  Synthetic oligonucleotide
caccatgagtccttcaaataccccgca

SEQ ID NO: 102 - DNA, 28 bp, Artificial
  Synthetic oligonucleotide
aaaccaatgcatttcttgatgattacgc

SEQ ID NO: 103 - DNA, 30 bp, Artificial
  Synthetic oligonucleotide
caccatgcaatcttcattagaaaaaaaaca

SEQ ID NO: 104 - DNA, 22 bp, Artificial
  Synthetic oligonucleotide
ctcctgaagacggcagcccgtc

SEQ ID NO: 105 - DNA, 30 bp, Artificial
  Synthetic oligonucleotide
caccatgcaaaaattcactgttgtaagcac

SEQ ID NO: 106 - DNA, 21 bp, Artificial
  Synthetic oligonucleotide
cttagcgtttttattgagtgc

SEQ ID NO: 107 - DNA, 27 bp, Artificial
  Synthetic oligonucleotide
caccatgagcagcgccgttgatgaaca

SEQ ID NO: 108 - DNA, 19 bp, Artificial
  Synthetic oligonucleotide
ctcgctggtttcgggcgca

SEQ ID NO: 109 - DNA, 29 bp, Artificial
  Synthetic oligonucleotide
caccatgcaatcgtttttggtggaagata

SEQ ID NO: 110 - DNA, 19 bp, Artificial
  Synthetic oligonucleotide
aaatgccgtgcccaacgta

SEQ ID NO: 111 - DNA, 26 bp, Artificial
  Synthetic oligonucleotide
caccatgcaaactgcacctgacgagc

SEQ ID NO: 112 - DNA, 27 bp, Artificial
  Synthetic oligonucleotide
ataggttttatctaaaaagaaatcgtc

SEQ ID NO: 113 - DNA, 31 bp, Artificial
  Synthetic oligonucleotide
caccatggttaatttaggtccaattcaggta

SEQ ID NO: 114 - DNA, 25 bp, Artificial
  Synthetic oligonucleotide
ccccaattttttcaataagttttgc

SEQ ID NO: 115 - DNA, 28 bp, Artificial
  Synthetic oligonucleotide
caccatggaattgatgagttaccgctgc

SEQ ID NO: 116 - DNA, 30 bp, Artificial
  Synthetic oligonucleotide
acgtgctttacacattttataaaggatttc

SEQ ID NO: 117 - DNA, 33 bp, Artificial
  Synthetic oligonucleotide
caccatgcaagaatctttagaatcattgcgcga

SEQ ID NO: 118 - DNA, 22 bp, Artificial
  Synthetic oligonucleotide
taaatccaattgcgttccaatg

SEQ ID NO: 119 - DNA, 27 bp, Artificial
  Synthetic oligonucleotide
caccatgatcggctccttagctgataa

SEQ ID NO: 120 - DNA, 19 bp, Artificial
  Synthetic oligonucleotide
acgagatacgggaaccggc

SEQ ID NO: 121 - DNA, 28 bp, Artificial
  Synthetic oligonucleotide
caccatgatgccgattgccattggcggc

SEQ ID NO: 122 - DNA, 25 bp, Artificial
  Synthetic oligonucleotide
attgctgatggtgaataaagtaatc

SEQ ID NO: 123 - DNA, 30 bp, Artificial
  Synthetic oligonucleotide
caccatgcatgatttatgggtttggggtga

SEQ ID NO: 124 - DNA, 30 bp, Artificial
  Synthetic oligonucleotide
tttaacttcaaatactaaagttgctttatc

SEQ ID NO: 125 - DNA, 28 bp, Artificial
  Synthetic oligonucleotide
caccatgattccaaattatcagcgttac

SEQ ID NO: 126 - DNA, 21 bp, Artificial
  Synthetic oligonucleotide
ccgtccccaacaacgagtaga

SEQ ID NO: 127 - DNA, 30 bp, Artificial
  Synthetic oligonucleotide
caccatggcggacgcgaaaaaagataaagc

SEQ ID NO: 128 - DNA, 26 bp, Artificial
  Synthetic oligonucleotide
cttcaaaacagattctttgatttcgc

SEQ ID NO: 129 - DNA, 28 bp, Artificial
  Synthetic oligonucleotide
caccatggatccgctggcgaattacaca

SEQ ID NO: 130 - DNA, 22 bp, Artificial
  Synthetic oligonucleotide
aaaacgcacttcttcggggagc

SEQ ID NO: 131 - DNA, 27 bp, Artificial
  Synthetic oligonucleotide
caccatgggttgccattcttacccctc

SEQ ID NO: 132 - DNA, 24 bp, Artificial
  Synthetic oligonucleotide
ataccttggtgaagttccgatata

SEQ ID NO: 133 - DNA, 26 bp, Artificial
  Synthetic oligonucleotide
caccatgaccacaacttatccgcaaa

SEQ ID NO: 134 - DNA, 21 bp, Artificial
  Synthetic oligonucleotide
atgagaagagtggcgcaactg

SEQ ID NO: 135 - DNA, 32 bp, Artificial
  Synthetic oligonucleotide
caccatgcaaaatccgttatttttatatcgtc

SEQ ID NO: 136 - DNA, 21 bp, Artificial
  Synthetic oligonucleotide
ttgagtggtcagcggcactgc

SEQ ID NO: 137 - DNA, 28 bp, Artificial
  Synthetic oligonucleotide
caccatgcagccgcaattaaccttatta

SEQ ID NO: 138 - DNA, 23 bp, Artificial
  Synthetic oligonucleotide
gaggcgcttaatcgatgattttg

SEQ ID NO: 139 - DNA, 29 bp, Artificial
  Synthetic oligonucleotide
caccatgacgaccggttcattattagaac

SEQ ID NO: 140 - DNA, 22 bp, Artificial
  Synthetic oligonucleotide
caaacgggtttttaaacgcgtc

SEQ ID NO: 141 - DNA, 30 bp, Artificial
  Synthetic oligonucleotide
caccatggataacaaaaaagaatctcaaga

SEQ ID NO: 142 - DNA, 20 bp, Artificial
  Synthetic oligonucleotide
cttcatcggaattggcgcag

SEQ ID NO: 143 - DNA, 27 bp, Artificial
  Synthetic oligonucleotide
caccatggaaactatggtcaactatgc

SEQ ID NO: 144 - DNA, 28 bp, Artificial
  Synthetic oligonucleotide
caaagttaaactccagctcttaatatag

SEQ ID NO: 145 - DNA, 30 bp, Artificial
  Synthetic oligonucleotide
caccatgatgcaaaatcagcaattatctga

SEQ ID NO: 146 - DNA, 18 bp, Artificial
  Synthetic oligonucleotide
ttggatcgcttttgcgcc

SEQ ID NO: 147 - DNA, 29 bp, Artificial
  Synthetic oligonucleotide
caccatgtccaagccttataacatggcgc

SEQ ID NO: 148 - DNA, 22 bp, Artificial
  Synthetic oligonucleotide
ataaataatcaccacacgccga

SEQ ID NO: 149 - DNA, 27 bp, Artificial
  Synthetic oligonucleotide
caccatgaatcaatcgaaagcagatgc

SEQ ID NO: 150 - DNA, 20 bp, Artificial
  Synthetic oligonucleotide
gcgggcggtgaaatctcgcg

SEQ ID NO: 151 - DNA, 27 bp, Artificial
  Synthetic oligonucleotide
caccatgtcctttgttcctgattacga

SEQ ID NO: 152 - DNA, 27 bp, Artificial
  Synthetic oligonucleotide
tttaatatcaggtaattgcgcgccgta

SEQ ID NO: 153 - DNA, 26 bp, Artificial
  Synthetic oligonucleotide
caccatgggtggcagcagcgaggaat

SEQ ID NO: 154 - DNA, 20 bp, Artificial
  Synthetic oligonucleotide
aggacgtttggcgccgaatc

SEQ ID NO: 155 - DNA, 29 bp, Artificial
  Synthetic oligonucleotide
caccatggcacaagaagcttatcaagtgc

SEQ ID NO: 156 - DNA, 27 bp, Artificial
  Synthetic oligonucleotide
aaatgttttgctcaattcaatataagc

SEQ ID NO: 157 - DNA, 27 bp, Artificial
  Synthetic oligonucleotide
caccatgtcgggaatgcagttggatca

SEQ ID NO: 158 - DNA, 25 bp, Artificial
  Synthetic oligonucleotide
taatttttttattgagcttccgcgc

SEQ ID NO: 159 - DNA, 25 bp, Artificial
  Synthetic oligonucleotide
caccatggatgatgccgtcattgaa

SEQ ID NO: 160 - DNA, 30 bp, Artificial
  Synthetic oligonucleotide
taaacgcgtttctaaaattttccgcaaatc

SEQ ID NO: 161 - DNA, 29 bp, Artificial
  Synthetic oligonucleotide
caccatgttgccgctgaaagaagatttta

SEQ ID NO: 162 - DNA, 20 bp, Artificial
  Synthetic oligonucleotide
taaagcggcaggaccagaac

SEQ ID NO: 163 - DNA, 27 bp, Artificial
  Synthetic oligonucleotide
caccatgcacattatggcgccggaaag

SEQ ID NO: 164 - DNA, 29 bp, Artificial
  Synthetic oligonucleotide
ttttttaacggtatgcgtttttttcagag

SEQ ID NO: 165 - DNA, 30 bp, Artificial
  Synthetic oligonucleotide
caccatggtgaccgtgaatatttattttcc

SEQ ID NO: 166 - DNA, 21 bp, Artificial
  Synthetic oligonucleotide
tttttgttgccacgaaccgtc

SEQ ID NO: 167 - DNA, 32 bp, Artificial
  Synthetic oligonucleotide
caccatgtcagaagaaactaaaaaagaacctc

SEQ ID NO: 168 - DNA, 27 bp, Artificial
  Synthetic oligonucleotide
ttgaacggatttttccgcttctttaac

SEQ ID NO: 169 - DNA, 28 bp, Artificial
  Synthetic oligonucleotide
caccatgcttcccacgccaccagacatc

SEQ ID NO: 170 - DNA, 25 bp, Artificial
  Synthetic oligonucleotide
ttctttaatacccaattgatccagc

SEQ ID NO: 171 - DNA, 28 bp, Artificial
  Synthetic oligonucleotide
caccatggacgcgtattgcgatgcatac

SEQ ID NO: 172 - DNA, 22 bp, Artificial
  Synthetic oligonucleotide
gctaccccatttgttgatgatg

SEQ ID NO: 173 - DNA, 27 bp, Artificial
  Synthetic oligonucleotide
caccatggaaatctataccgtcggcac

SEQ ID NO: 174 - DNA, 19 bp, Artificial
  Synthetic oligonucleotide
ttcgagccatttatccacg

SEQ ID NO: 175 - DNA, 30 bp, Artificial
  Synthetic oligonucleotide
caccatgagctacacttttcaaaatcttcc

SEQ ID NO: 176 - DNA, 23 bp, Artificial
  Synthetic oligonucleotide
agcgtttgccgcagcaaataatt

SEQ ID NO: 177 - DNA, 26 bp, Artificial
  Synthetic oligonucleotide
caccatggaagaagctgccgtgccag

SEQ ID NO: 178 - DNA, 21 bp, Artificial
  Synthetic oligonucleotide
tttcaagccttttgctttgag

SEQ ID NO: 179 - protein, 328 aa, Dichelobacter nodosus
MKKTSLLLSAAIALSLTQVYAGVVLKNEGQKVGYAIGVDMGSSIAQLGISDGEELDFNAVILGLRDAYQKKDLLLTQDEMTKTLQDFSEKRLQAMKKEMEKIAAEEAEKGKAFLEENAKKDGVITTESGLQYKVVKKGTGAKPNSDDRVTVDYTGTLIDGTEFDSSKGREPITINVQDVIAGWVEGLQLMTEGANYIFYIPSDLAYGSRGAGNAIPPNATLIFDVNLLKIEKNEAEAEADKKESIAKSINKSLEEATEIVKAEVEADKKESIAKSINKSLEEATETVKAEAEADKKEAIANSINKSLEEAAEAVKEVIEAKPDEAAKK

SEQ ID NO: 180 - DNA, 990 bp, Dichelobacter nodosus
atgatgaaaaaaacttctttactattatcagccgcgattgccttgagtttgacgcaggtttatgccggcgttgtgttaaaaaatgaagggcaaaaagtgggttatgccatcggggtggatatgggaagttccattgcgcaattaggtatttctgatggggaagaattggattttaatgccgtgattttgggattgcgcgatgcttatcaaaagaaagatttgcttttaactcaagatgaaatgacgaaaacgctgcaagatttttctgaaaaacgtttgcaagccatgaaaaaagaaatggaaaaaatcgccgctgaagaagcagaaaaaggcaaagcgtttttagaagaaaatgcgaaaaaagacggcgtaattacaacggaatctggtttgcaatataaagttgttaaaaaaggaaccggcgcaaaacccaatagcgacgaccgcgttaccgttgattacaccggcactttgattgacggcaccgaatttgacagctcaaaaggacgcgaacctattacgattaacgtacaagatgttattgctggttgggttgaaggtttgcaactgatgaccgaaggcgctaattatattttctatattccgtctgatttagcttatggctctcgcggcgccggtaacgctattccgcctaatgctactttgatttttgatgtgaatttactcaaaattgaaaaaaatgaagcggaagccgaagctgataaaaaagaatcgattgcaaaatccatcaataaatctttagaagaagccacggaaatcgtaaaagcagaagtggaagctgacaaaaaagaatcgattgcaaaatccattaataaatctttggaagaagccacagaaaccgtaaaagcggaagccgaagctgataaaaaagaagccattgccaattctattaataaatctttagaagaagcggcggaagcggtaaaagaagtgatcgaagcgaaacctgatgaagcggctaaaaaatag

SEQ ID NO: 181 - protein, 246 aa, Dichelobacter nodosus
MKKLLLAALLSCSLPCLAADKYRVASHPTFAPYEFLDGNGIITGYDVDLIQEIAKDQGFEVEIHNDKWEELLDKLNDGKRDMIVSSLMDTEERRQLADLSKPYSEYEQFTVFYKRPDLSINSLSDLSGLNAGAEKGTANVERLKQAGANVTELDSNFEGFKAIIRGSIDAYYCDEASMAYILRGYKDKKLPIRTFRLPKEPGKTVIAVKKGNAELLEKINRGIDNLRANGKLDELRKKWLEMESAN

SEQ ID NO: 182 - DNA, 741 bp, Dichelobacter nodosus
atgaaaaaattattattggcagcattattgtcatgtagtttgccctgtttggcagcggataaatatcgcgttgctagtcatccgactttcgcaccgtatgagtttttagacggaaacggcatcatcaccggatatgacgttgatctcattcaagaaattgccaaagatcaaggctttgaagttgaaattcataatgataaatgggaagaactgcttgataaattaaatgacggcaaacgcgatatgatcgtttcttccttgatggatactgaagaacggcgccaattggctgatttgagtaagccttattctgaatatgaacaatttaccgtgttttataaaagacccgatctgtcaatcaattctttatccgatttgtccggtttaaatgctggtgcggaaaaaggtacggcaaacgttgaacggctgaaacaagctggcgcaaatgttactgaattggatagtaattttgaaggatttaaagccattattcggggttctattgatgcttactattgcgatgaagcatcgatggcttatattttgagaggatataaagataaaaaattgccgattcgcacttttagattgccgaaagaaccgggaaaaaccgttatcgcagttaaaaaaggcaacgcggaattgctcgaaaaaatcaaccgtggcatcgataatttgcgcgcaaatggaaaactcgatgagcttcgtaaaaaatggttggaaatggaatccgctaattaa

SEQ ID NO: 183 - protein, 595 aa, Dichelobacter nodosus
MKQSGINGVKTLTLVVCAALASQAYAAVNYESANYIGSQPEGSVRFIIKYKDKSQSQQMMTNRSTTSVMNNNNITIAGFNAQFVRTMTIGAGIFAVPDLKTTKEAHLVMDTIASNPDVEYVEVDRWLRPFAAPNDPFYNDQWHYYSEYGVKADKVWDRGITGKGVTVAVVDTGIVNHPDLNANVIPGSGYDFIQEAEIAQDGDGRDSNPADAGDWHSNWACGKYPDPRYEKRNSSWHGSHVAGTIAAVTNNRIGVSGVAYDAKIVPVRVLGRCGGYNSDINEGMYWAAGGHIDGVPDNKHPAQVINMSLGGPGVCGSTEQTLINRATQLGATIIVAAGNDNIDAYGVTPASCDNILTVGATTSNGTRAYFSNHGSVVDISAPGAGITSTVDSGARYPSGPSYSLMDGTSMATPHVAGVAALVISAANSVNKEMTPAQVRDVLVRTVSSFNGTPDRRIGAGIVDADAAVNAVLDGNVVERPIDELKPQAEYRNPQIKLIRDYQMMFSEIKVNGRPGNTKFAVVKADIRHTDPSQLKLRLVSPKGYEYAVHYDNIKNKSSELITFPRDEQMNGYWRLKIVDTKRGVTGYTRGWSVAF

SEQ ID NO: 184 - protein, 603 aa, Dichelobacter nodosus
MNLSNISAVKVLTLVVSAAIAGQVCAAESIVNYESANAISKQPEGSVRFIVKYKDGTPSSQGLKTRSTTKVMASGMQVAGFEAQFVRTTGLGAGIFAVPELKTTKEAHLVMDTIASNPDVEFVEVDRLAYPKAAPNDPSYRQQWHYFGNYGVKANKVWDRGFTGQGVVVSVVDTGILDHVDLNGNMLPGYDFISSAPNARDGDQRDNNPADEGDWFDNWDCGGYPDPRREKKFSTWHGSHVAGTIAAVTNNGVGVAGVAYGAKVIPVRVLGKCGGYDSDITDGMYWSAGGHIDGVPDNQNPAQVVNMSLGGGGGCSQNSQRMIDKTTNLGALIVIAAGNENQDASRTWPSSCNNVLSVGATTPKGKRAPFSNYGARVHLAAPGTNILSTIDVGQAGPVRSSYGMKAGTSMAAPHVSGVAALVISAANSIGKTLTPSELSDILVRTTSRFNGRLDRGLGSGIVDANAAVNAVLGDQNRAQPRPPVNQPINSGNKVYRSDRRVAIRDLRSVTSGIRVNDQARVGSANITLTLDIRHGDRSQLAVELIAPSGRVYPIYHDGKRQPNIVGPATFSVKNERLQGTWTLKVTDKARGVTGSIDSWSLTF

SEQ ID NO: 185 - protein, 300 aa, Dichelobacter nodosus
MKKSFLNSMMVLILTFFCSAAMAQKFTVVSTFTVIADIAQNIAGEHADVFSITKAGAEIHDYEPTPHDLVTAQKAQLLLHNGLQLERWFERFHQSLKNVPAVTVSAGVVPIPIEGDSGIPNPHAWMSIDNALIYVDNITQALQKYDPEHANDYAENARRYKEKIQALDTHLQQAFAKIPEQQRWLVTTEGAFSYLARDYHLREAYVWPVNSEQEGTPQQIADLIATVRKHQIPVVFSESTLSDKHAKVVAKETGAQYGGVLYVDSLSTADGAVPTYLDLLKVTTDTIISGFESALNKNAK

SEQ ID NO: 186 - DNA, 903 bp, Dichelobacter nodosus
atgaaaaaatcatttcttaactcaatgatggtactcattttgacgtttttttgctcggcagcgatggcgcaaaaattcactgttgtaagcacattcaccgtcattgctgatattgctcaaaatattgctggtgaacacgctgatgttttttctattactaaagctggcgctgaaatccacgattacgaacctacgcctcatgatttggttaccgcccaaaaagcgcaacttttattacataatggattgcaactagaacgttggtttgagcgttttcatcaatcattaaaaaatgtgccagcagtaacagtcagtgcgggggtagtacccattcctattgaaggtgattctggcattcctaatccacatgcatggatgtcgatcgataatgctttgatttatgttgataatatcactcaagcactgcaaaaatatgatcctgaacacgccaatgattacgctgaaaatgcacggcgttacaaagaaaaaattcaagcgcttgatacgcatcttcagcaagcttttgccaaaattccggaacaacaacgttggttagtaaccacagaaggcgctttttcctatttagcacgcgattatcaccttcgtgaagcctatgtgtggccggttaactctgagcaagaaggcactccacaacaaattgccgatttaatagcaactgtacgaaaacatcaaattcctgtcgtatttagcgaaagtaccctgtcagataaacatgcaaaagttgttgctaaggaaactggtgctcaatatggcggcgttttatacgttgattcactttctactgctgatggcgcggtacctacttatttagatttattgaaagtaactaccgatacaataatcagcggttttgaatctgcactcaataaaaacgctaagtaa

SEQ ID NO: 187 - protein, 230 aa, Dichelobacter nodosus
MKKEQFLAAALLVFGFSTAAHAELMSYRCDQDRKIDVVYSFNSAGVPTKAEVMIQGKKRVLNYNMDRSDNVDSFFNDKAGYELSSNIITRDDYHSSSIMIMSPKSEILFKNCSAAMSTIQPVMSEDAPSVDANVVTSVKQANYVCQQGKKMKVEYGFNEVGIPVYALVNFKGTKLKLPYDLDQSSEASTFFTKDGYVFAGDGMTVDTYRNSFMMLTAPSNEILYKMCKAR

SEQ ID NO: 188 - DNA, 903 bp, Dichelobacter nodosus; identical to SEQ ID NO: 186

SEQ ID NO: 189 - protein, 596 aa, Dichelobacter nodosus
MNKMALVVCAALVGQVASAETMVNYASAKAIGKQPAGSVRFIVKYKDNSQSSKDLKNRSTTKVMANGMQVAGFNAQFVRMTGAGAGIFSVPDLKTTKEAHLVMDTIASNPDVEFVEVDRIARPTAAPNDQHYREQWHYFDRYGVKADKVWDMGFTGQNVVVAVVDTGILHHRDLNANVLPGYDFISNSQISLDGDGRDADPFDEGDWFDNWACGGYPDPRKERSDSSWHGSHVAGTIAAVTNNRIGVAGVAYGAKVVPVRALGRCGGYDSDISDGLYWAAGGRIAGIPENRNPAKVINMSLGSDGQCSYNAQTMIDRATRLGALVVVAAGNENQNASNTWPTSCNNVLSVGATTSRGIRASFSNYGVDVDLAAPGQDILSTVDSGTRRPVSDAYSFMAGTSMATPHVSGVAALVISAANSVNKNLTPAELKDVLVSTTSPFNGRLDRALGSGIVDAEAAVNSVLGNEGNNGRDDRRDNVAPVENARNYANNSIKFIRDYRLTSSVIEVEGRSGAANGKINLALDIRHGNRSQLSIQLTSPAGHVYHINHDGARRPNLSGTVEIPVQNEQINGAWVLQVGDHGRGATGYIKSWSLTL

SEQ ID NO: 190 - protein, 489 aa, Dichelobacter nodosus
MFTGNKFTVSVLSALTLAILTACGGSSEEFTLDRDHKWSHEPDRAKKEKRKHAELPAVNIPAPTSFLEGAKKMKQTDDDNKEDKAVSKRQEGNVVTVVDHDYGYKTVKFNQSGMVDHLCMRVSESGVCKIDDNKVANGIKNKEKEKKAAEGELAKIQKSIDDYMKTWDENHKNAEFGEREKELERIKELNKISDIEDRIEILEEELNELNEANKNMVFFLKSDGTQRLPNTSERILTKENYKNSYSKEKDIEDAGIVPVAGFYDKNGDLRVQKGMVGREFDGIIVLENEVNQKKEVVKRSYLRDPVAAGWSYNTFGVFQDSTKSIERGYQSIGVKTRTSAQKRKEADESFEVDKGYENLPYYGRATYTGIGHAYHNDEQVTMNVKVDADFHDRHLNFETSNATAHTFEKEGHLTRVRPDLDLKGAADWAADVADFKGKVHNVGGNLEGQLEGSFYGPKAPEVGGVYGLHGKDKEGKPVNYVGGFGAKRP

SEQ ID NO: 191 - DNA, 1470 bp, Dichelobacter nodosus
atgtttacaggaaataagtttacagtttcagtgttatcagctttaacgctggcaattttgacggcatgcggtggcagcagcgaggaattcaccctagatagagatcataaatggtcacatgaaccagatcgcgcaaaaaaagaaaaaagaaaacatgcggaattaccggcggttaatatccctgcgccaacaagttttttagagggcgctaaaaaaatgaaacaaactgatgacgataataaagaagataaagcagtttcaaagagacaagaaggcaacgtggtgactgtggttgatcatgattatggttataaaacagttaagtttaatcaatcaggtatggttgaccatttatgtatgagagtaagtgaatcaggtgtatgcaagattgatgataacaaagttgctaacggtatcaaaaataaggaaaaagagaaaaaagctgcggaaggggaactcgctaaaattcagaaaagtattgatgactacatgaagacatgggatgagaatcataaaaacgctgaatttggtgagcgtgaaaaagagcttgagcggataaaggaactgaataagatcagtgatattgaagatcgtattgaaatattagaagaagaactaaatgagctgaatgaagcaaataaaaatatggttttctttttaaaatcagatggaacacaacgtcttccaaatacatcagagagaatcttgacaaaagagaactataaaaatagttacagcaaagaaaaagatattgaagatgctggtattgttcccgttgctggattttacgataaaaatggcgatttaagagtgcaaaagggcatggttggtagagaattcgatggcattatcgtgcttgaaaatgaagtgaatcaaaaaaaagaagtggttaaacgctcttacttacgcgatccggtagcggcaggctggagttataacacttttggcgttttccaagattcaacgaagagtattgaacgcggttaccaaagtattggtgttaaaacccgaaccagtgctcaaaaaagaaaagaagctgacgaaagctttgaagttgataaaggatatgaaaacttaccctattatggacgcgcaacctataccggtatcggtcatgcttatcacaatgatgagcaagtgacgatgaatgtaaaagttgatgctgatttccatgatcgtcatttaaatttcgaaacttcaaatgccactgcgcatacttttgaaaaagaaggtcatttgacacgtgttcgtcctgatttagatttaaaaggcgctgcggattgggcagctgatgttgccgattttaaaggtaaagtgcataacgtaggcggcaacttagaaggtcaattggaaggcagtttctatggaccaaaagcgcctgaagtgggcggcgtttatggtttacacggcaaagataaagaaggtaaaccagttaactatgttggcggattcggcgccaaacgtccttga

SEQ ID NO: 192 - DNA, 18 bp, Artificial
  Synthetic oligonucleotide
cggcgtcgtttgatggat

SEQ ID NO: 193 - DNA, 18 bp, Artificial
  Synthetic oligonucleotide
acggtgcagggcgaataa

SEQ ID NO: 194 - DNA, 22 bp, Artificial
  Synthetic oligonucleotide
catggttgaagtgccaaatgtt

SEQ ID NO: 195 - DNA, 20 bp, Artificial
  Synthetic oligonucleotide
cgatccccggcatatttttt

SEQ ID NO: 196 - DNA, 16 bp, Artificial
  Synthetic oligonucleotide
cgtggtgcgcgtcgat

SEQ ID NO: 197 - DNA, 20 bp, Artificial
  Synthetic oligonucleotide
tgtttgcgtagcgcttcaat

SEQ ID NO: 198 - DNA, 20 bp, Artificial
  Synthetic oligonucleotide
ccggctttggtaatttcgaa

SEQ ID NO: 199 - DNA, 19 bp, Artificial
  Synthetic oligonucleotide
tctccggttttgggattgc

SEQ ID NO: 200 - DNA, 16 bp, Artificial
  Synthetic oligonucleotide
cgcgagcgcattttgc

SEQ ID NO: 201 - DNA, 19 bp, Artificial
  Synthetic oligonucleotide
tggcgctcatcgattgatt